(19) United States
(12) Patent Application Publication
(10) Pub. No.: US 2026/0347419 A1
(43) Pub. Date: Nov. 5, 2026

(54) AAV CAPSID CHIMERIC ANTIGEN RECEPTORS AND USES THEREOF

(71) Applicant: University of Massachusetts, Westborough, MA (US)

(72) Inventors: Allison Keeler-Klunk, Worcester, MA (US); Terence Flotte, Worcester, MA (US); Motahareh Arjomandnejad, Worcester, MA (US)

(73) Assignee: University of Massachusetts, Westborough, MA (US)

(21) Appl. No.: 19/657,083

(22) Filed: Apr. 24, 2026

Related U.S. Application Data
(62) Division of application No. 17/605,615, filed on Apr. 23, 2020, now Pat. No. 12,637,497.
(60) Provisional application No. 62/838,288, filed on Apr. 24, 2019.

(51) Int. Cl. C07K 14/47; A61K 40/11; A61K 40/22; A61K 40/31; A61K 40/42; A61K 40/46; C07K 14/005; C07K 14/705; C12N 15/62; C12N 15/86

(52) U.S. Cl. CPC C07K 14/4702; A61K 40/11; A61K 40/22; A61K 40/31; A61K 40/42; A61K 40/4221; A61K 40/46; C07K 14/005; C07K 14/70503; C07K 14/7051; C07K 14/70517; C07K 14/70521; C07K 14/70578; C12N 15/62; C12N 15/86; A61K 2239/31; A61K 2239/38; A61K 2239/48; C07K 2317/622; C07K 2319/02; C07K 2319/03; C07K 2319/33; C07K 2319/50; C12N 2740/16043; C12N 2750/14134

(57) ABSTRACT

Aspects of the disclosure relate to compositions and methods for modulating the immune response of a subject (e.g., a human subject) to certain viral antigens (e.g., antigens derived from AAV capsid proteins). The disclosure is based, in part, on isolated nucleic acids and expression constructs encoding chimeric antigen receptors (CARs) that target AAV capsid proteins, and recombinant immune cells comprising such constructs (e.g., recombinant T-cell comprising a CAR (CAR T-cells), and recombinant T-regulatory cells comprising a CAR (CAR T-regs)).

### RELATED APPLICATIONS

This application is a divisional of U.S. application Ser. No. 17/605,615, filed Oct. 22, 2021, which is a national stage filing under 35 U.S.C. § 371 of international PCT application PCT/US2020/029527, filed Apr. 23, 2020, which claims priority under 35 U.S.C. § 119(e) to U.S. provisional patent application, U.S. Ser. No. 62/838,288, filed Apr. 24, 2019, the entire contents of each of which are incorporated by reference herein.

### STATEMENT REGARDING FEDERALLY SPONSORED RESEARCH OR DEVELOPMENT

This invention was made with government support under grant number HL131471 awarded by the National Institutes of Health. The government has certain rights in the invention.

### REFERENCE TO AN ELECTRONIC SEQUENCE LISTING

The contents of the electronic sequence listing (U012070119US03-SEQ-KZM.xml; Size: 120,712 bytes; and Date of Creation: Apr. 22, 2026) are herein incorporated by reference in their entirety.

### BACKGROUND OF THE INVENTION

Clinical trials with recombinant Adeno-associated virus (rAAV) have been observed to elicit T-cell mediated immune responses against the Adeno-associated virus (AAV) capsid of the recombinant virus. Different responses have been observed. For example, in trials for hemophilia where rAAVs were injected into the hepatic artery, a cytotoxic CD8+ T-cell response against capsid was responsible for elevated liver enzymes and clearance of transfected cells. In other clinical trials, for example alpha-1-anti-trypsin deficiency and lipoprotein lipase deficiency, where the injection site was intramuscular, a CD4+ T-regulatory response was observed with long-term expression of transgene without steroids.

### SUMMARY OF THE INVENTION

Embodiments of the disclosure relate to compositions and methods for modulating the immune response of a subject (e.g., a human subject) to certain viral antigens (e.g., AAV capsid proteins or antigens derived from AAV capsid proteins). The disclosure is based, in part, on isolated nucleic acids and expression constructs encoding chimeric antigen receptors (CARs), and recombinant immune cells comprising such constructs (e.g., recombinant T-cell comprising a CAR (CAR T-cells), and recombinant T-regulatory cells comprising a CAR (CAR T-regs)). In some embodiments, CAR T-cells described herein are useful for killing immune cells (e.g., antigen presenting cells (APCs)) which induce immune responses against AAV capsid proteins, thereby dampening (e.g., inhibiting or suppressing) the host immune response against the AAV capsid protein). In some embodiments, CAR T-reg cells described herein are useful for inhibiting the killing of immune cells (e.g., APCs) which induce immune responses against AAV capsid proteins, thereby increasing (e.g., promoting or inducing) the host immune response against AAV capsid protein.

Accordingly, in some embodiments, the disclosure provides an isolated nucleic acid comprising an expression construct encoding a CAR, wherein the CAR comprises: an antigen binding domain that targets (e.g., specifically binds) one or more AAV capsid protein epitopes; a transmembrane domain; and a cytoplasmic signaling domain.

In some embodiments, the nucleic acid encodes a CAR with an antigen binding domain with a first portion comprising a monoclonal antibody or a single chain variable fragment (scFv). In some embodiments, the nucleic acid encodes an antigen binding domain comprising an scFv having a variable heavy chain domain (VH) derived from an AAV capsid protein-targeting antibody linked to a variable light chain domain (VL). In some embodiments, the nucleic acid encodes an antigen binding domain comprising an scFv having a variable heavy chain domain (VH) derived from an AAV capsid protein-targeting antibody linked to a variable light chain domain (VL) derived from an AAV capsid protein-targeting antibody.

In some embodiments, the nucleic acid encodes a VH domain and a VL domain linked by a linking molecule. In some embodiments, the linking molecule is a peptide linker. In some embodiments, the peptide linker is a glycine-rich linker (e.g., a (GGGGS)n linker, where n is an integer between 1 and 10).

In some embodiments, the nucleic acid encodes an antigen binding domain further comprising an IgG1 heavy chain constant domain 2 (IgG1 CH2) and/or an IgG1 heavy chain constant domain 3 (IgG1 CH3). In some embodiments, the IgG CH2 and/or the IgG1 CH3 domain are linked to a nucleotide sequence encoding an antibody or scFv by a linking molecule. In some embodiments, a linking sequence encodes a molecule comprising an IgG1 hinge linker and/or an IgG2 hinge linker.

In some embodiments, the nucleic acid encodes an antigen binding domain targeting (e.g., specifically binds to) an epitope of an AAV capsid protein having a serotype selected from AAV1, AAV2, AAV3, AAV3b, AAV4, AAV5, AAV6, AAV7, AAV8, AAVrh.8, AAV9, AAV10, AAVrh.10, AAVrh.39, AAVrh.43, AAV.PHPB, AAV.PHPB.e, AAVrh32.33, or a variant thereof. In some embodiments, the nucleic acid encodes an antigen binding domain targeting an AAV2 epitope or an AAV6 epitope.

In some embodiments, the nucleic acid encodes a CAR comprising a transmembrane domain which is a CD28 transmembrane domain. In some embodiments, the nucleic acid encodes a CAR comprising a cytoplasmic signaling domain comprising one or more of a CD28 signaling domain, a 4-1BB signaling domain, and a CD3 signaling domain. In some embodiments, the cytoplasmic domain comprises a CD28 signaling domain, a 4-1BB signaling domain, and a CD3 signaling domain.

In some embodiments, an expression construct further comprises a promoter operably linked to a nucleic acid sequence encoding a CAR. In some embodiments, a promoter is an EF1alpha (EF1α) promoter. In some embodiments, an expression construct further comprises a Woodchuck Hepatitis Virus Posttranscriptional Regulatory Element (WPRE). In some embodiments, an EF1alpha promoter is positioned 5′ to a nucleic acid sequence encoding a CAR, and a WPRE is positioned 3′ to the nucleic acid sequence encoding the CAR.

In some embodiments, an expression construct further comprises a forkhead box P3 (FoxP3) encoding nucleic acid sequence. In some embodiments, a FoxP3 encoding nucleic acid sequence is linked to a nucleic acid sequence encoding a CAR. In some embodiments, a FoxP3 encoding sequence and a nucleic acid sequence encoding a CAR are linked by a nucleic acid sequence encoding a 2A self-cleaving peptide.

In some embodiments, an expression construct is flanked by viral long terminal repeats (LTRs). In some embodiments, the LTRs are retroviral LTRs. In some embodiments, LTRs are HIV LTRs.

In some embodiments, the disclosure provides a vector comprising an isolated nucleic acid as described herein. In some embodiments, the vector is a plasmid. In some embodiments, the vector is a viral vector, for example a lentiviral vector, retroviral vector, or an adenoviral vector.

In some embodiments, the disclosure provides a host cell comprising an isolated nucleic acid or a vector as described herein. In some embodiments, the host cell comprises a CAR encoded by a nucleic acid as described herein. In some embodiments, a host cell is a mammalian cell. In some embodiments, a mammalian cell is a human cell.

In some embodiments, a host cell is an immune cell. In some embodiments, an immune cell is a T-cell or a regulatory T-cell (T-reg).

In some embodiments, the disclosure provides a composition comprising a host cell as described herein. In some embodiments, a composition comprises a plurality of host cells. In some embodiments, a composition further comprises a pharmaceutically acceptable excipient. In some embodiments, a pharmaceutically acceptable excipient is a cryoprotectant.

In some embodiments, the disclosure provides a method for modulating an immune response of a subject to an AAV capsid protein, the method comprising administering to the subject a host cell (e.g., a composition comprising a plurality of host cells) as described herein. In some embodiments, the host cells are autologous host cells (e.g., derived from the subject). In some embodiments, the host cells are heterologous (e.g., derived from a different subject of the same species as the subject) or xenogeneic (e.g., derived from a different species than the subject) host cells.

In some embodiments, a subject is a human. In some embodiments, a subject has previously been administered an rAAV, or is contemplated to be administered an rAAV (e.g., a therapeutic rAAV).

In some embodiments, the method comprises administering to the subject a CAR T-cell such that the immune response of the subject to the AAV capsid protein is inhibited relative to a control or a subject not having been administered the CAR T-cell.

In some embodiments, the method comprises administering to the subject a CAR T-reg such that the immune response of the subject to the AAV capsid protein is induced relative to a control or a subject not having been administered the CAR T-reg.

In some embodiments, the methods further comprise administering a therapeutic rAAV to the subject. In some embodiments, the rAAV is administered in combination with a CAR T-cell. In some embodiments, the rAAV is administered prior to the administration of a CAR T-cell. In some embodiments, the rAAV is administered after the administration of a CAR T-cell. In some embodiments, the therapeutic rAAV comprises a capsid protein having the same serotype as the capsid protein targeted by the CAR.

In some embodiments, the disclosure relates to a vector carrying a nucleic acid encoding a CAR. In some embodiments, the vector is a lentiviral vector. In some embodiments, the lentiviral vector comprises a nucleic acid encoding a CAR having the sequence as set forth in SEQ ID NO: 1. In some embodiments, the lentiviral vector comprises a nucleic acid encoding a CAR having the sequence as set forth in SEQ ID NO: 2. In some embodiments, the lentiviral vector comprises a nucleic acid encoding a CAR having the sequence as set forth in SEQ ID NO: 3. In some embodiments, the lentiviral vector comprises a nucleic acid encoding a CAR having the sequence as set forth in SEQ ID NO: 4.

These and other aspects and embodiments will be described in greater detail herein. The description of some exemplary embodiments of the disclosure are provided for illustration purposes only and not meant to be limiting. Additional compositions and methods are also embraced by this disclosure.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, Drawings, Examples, and Claims.

### BRIEF DESCRIPTION OF DRAWINGS

FIGS. 1A-1B are schematics depicting two embodiments of CAR T-cell constructs that recognize (e.g., specifically bind to) AAV2 capsid protein (e.g., SEQ ID NO: 1-2).

FIGS. 2A-2B are schematics depicting two embodiment of third generation CAR T and CAR T-reg cells (e.g., SEQ ID NO: 16-17). FIG. 2A: 3rd generation Lentiviral vector for CAR-Regulatory-T-cells. Vector contains ScFv anti-AAV capsid derived from D3 antibody with reactivity against major AAV capsid variants, human CD28 domain, human 4-1BB domain, human CD3z domain. Included after a self-cleavage domain are FoxP3 transcription factor and CD19 truncated extracellular domain. Different versions include: murine and human genes, shorter Fc linker, 2nd generation CARs with either CD28 domain or 41BB domain, other co-stim, other reporter genes (CD19 or EGFR). FIG. 2B: 3rd generation Lentiviral vector for CAR-T-cells. Vector contains ScFv anti-AAV capsid derived from D3 antibody with reactivity against major AAV capsid variants, human CD28 domain, human 4-1BB domain, human CD3z domain. Included after a self-cleavage domain is CD19 truncated extracellular domain. Different versions include: murine and human genes, shorter Fc linker, 2nd generation CARs with either CD28 domain or 41BB domain, other co-stim, other reporter genes (CD19 or EGFR).

FIGS. 3A-3B show schematics depicting embodiments of CAR T-cell and CAR T-reg cell constructs (e.g., SEQ ID NO: 1-2 and 16-17).

FIG. 4 shows representative Fluorescence Assisted Cell Sorting (FACS) data of CARs expression on the surface of cells using an antibody to detect the CH2-CH3 constant domain of the CAR.

FIG. 5 is a schematic depicting one embodiment of a CAR T-cell expansion protocol. Representative FACS data indicating increased CAR T-cell population is also shown.

FIG. 6 shows representative FACS data for transfection efficiency of Raji-fluc cells with GFP-AAV6 and T-cells with CAR constructs.

FIG. 7 shows representative data indicating that AAV-CAR constructs kill AAV-transfected Raji-fFluc+ cells.

FIG. 8 shows representative data indicating the antigen-specific killing activity of AAV-CAR T-cells.

FIG. 9 shows representative data indicating that CAR T-regs (AAV-CAR-FoxP3) inhibit antigen specific killing by AAV-CAR T-cells.

FIGS. 10A-10G. FIG. 10A: graphical schematic of AAV-CAR-T-cells and AAV-CAR-Regulatory-T-cells expansion. FIG. 10B: Representative flow cytometry plots of transfected cells before and after expansion. FIG. 10C: Flow cytometry plots of IL-2 production by AAV-CAR-T-cells after different stimulation conditions. FIG. 10D: IL-2 concentration of AAV-CAR-T-cells after different stimulation conditions by ELISA. FIG. 10E: Flow cytometry plots of IFN-γ production by AAV-CAR-T-cells after different stimulation conditions. FIG. 10F: Flow cytometry plots of IFN-γ production by AAV-CAR-T-cells co-cultured with AAV-CAR-Regulatory-T-cells after different stimulation conditions. FIG. 10G: IFN-γ concentration of AAV-CAR-T-cells with or without AAV-CAR-Regulatory-T-cells after different stimulation conditions by ELISA.

FIGS. 11A-11E. FIG. 11A: Graphical schematic of a luciferase killing assay and inhibition of luciferase killing assay. FIG. 11B: Quantitative results of killing assay and inhibition of killing assay for AAV1, AAV6, AAV3b and Rh32.33 capsid variants. FIG. 11C: Graphical schematic of a luciferase killing assay and inhibition of luciferase killing assay. FIG. 11D: Quantitative results of killing assay and inhibition of killing assay against AAV6 transfected-Raji cells (CD20+) and Raji cells (CD20+) using AAV-CAR-T-cells and CD20-CAR-Tregs. FIG. 11E: Quantitative results of killing assay and inhibition of killing assay against AAV6 transfected-Raji cells (CD20+) and Raji cells (CD20+) using CD20-CAR-T-cells and AAV-CAR-Tregs.

FIGS. 12A-12F. FIG. 12A: Graphical schematic of in vivo CAR-T-cell response. Intramuscular injections of AAV1-human AAT [5*10¹⁰ viral titer]. At week 3, intravenous injection of AAV-CAR-T-cells [5*10⁶ cells] or PBS followed by intraperitoneal of IL-2 [45000 IU] for 3 days. FIG. 12B: Time course of serum human AAT protein levels of control animals and animals that received AAV-CAR-T-cells (left). Normalized human AAT protein levels to the baseline at week 3 for control animals and animals that received AAV-CAR-T-cells (right). Arrows represents delivery of CAR T-cell delivery. FIGS. 12C-F: Flow cytometry plots of isolated T-cells from the muscles of AAV1-human AAT injected animals or AAV1-human AAT injected animals with AAV-CAR-T-cells. Populations of CD3+ (FIG. 12C); CD3+, CD8+, or CD4+ (FIG. 12D); CD3+, CD90.2+ (FIG. 12E); CD3+, CD90.2+, CD19+ (FIG. 12F).

FIGS. 13A-13F. FIG. 13A: Graphical schematic of in vivo CAR-Regulatory-T-cell suppression immune response against AAV-Rh32.33. Intra muscular injections of AAV-Rh32.33-human AAT [5*1010 viral titer]. At week 1, intravenous injection of AAV-CAR-Regulatory-T-cells [5*106 cells] or natural expanded regulatory T-cells [5*106 cells] or PBS followed by Intraperitoneal of IL-2 [45000 IU] for 3 days. FIG. 13B: Time course of serum human AAT protein levels of control animals and animals that received AAV-CAR-Regulatory-T-cells and expanded natural regulatory-T-cells (left). Normalized human AAT protein levels to the baseline at week 1 for control animals and animals that received AAV-CAR-Regulatory-T-cells and expanded natural regulatory-T-cells (right). Red arrow represents CAR T-Reg delivery. FIG. 13C: Graphical schematic of in vivo CAR-Regulatory T-cell suppression immune response against Ovalbumin-AAV1 in C57BL/6 animals. Intra muscular injections of Ovalbumin-AAV1 [5*1010 viral titer]. At week 1, intravenous injection of AAV-CAR-Regulatory-T-cells [5*106 cells] or natural expanded regulatory T-cells [5*106 cells] or PBS followed intraperitoneal of IL-2 [45000 IU] for 3 days. FIG. 13D: Time course of serum Ovalbumin levels of control animals and animals that received AAV-CAR-Regulatory-T-cells and expanded natural regulatory-T-cells (left). Normalized Ovalbumin protein levels to the baseline at week 1 for control animals and animals that received AAV-CAR-Regulatory-T-cells and expanded natural regulatory-T-cells (right). Red arrow represents CAR T-Reg delivery. FIG. 13E: Graphical schematic of in vivo CAR-Regulatory-T-cell suppression immune response against chimpanzee AAT-AAV1 in Balb/C animals. Intra muscular injections of chimpanzee AAT-AAV1 [5*1010 viral titer]. At week 1, intravenous injection of of AAV-CAR-Regulatory-T-cells [5*106 cells] or natural expanded regulatory T-cells [5*106 cells] or PBS followed by intraperitoneal of IL-2 [45000 IU] for 3 days. FIG. 13F: Time course of serum chimpanzee AAT protein levels of control animals and animals that received AAV-CAR-Regulatory-T-cells and expanded natural regulatory-T-cells (left). Normalized chimpanzee AAT protein levels to the baseline at week 1 for control animals and animals that received AAV-CAR-Regulatory-T-cells and expanded natural regulatory-T-cells (right). Red arrow represents CAR T-reg delivery.

### DETAILED DESCRIPTION OF CERTAIN EMBODIMENTS

Embodiments of the disclosure relate to compositions and methods for modulating the immune response of a subject (e.g., a human subject) to certain viral antigens (e.g., antigens derived from AAV capsid proteins). The disclosure is based, in part, on isolated nucleic acids and expression constructs encoding chimeric antigen receptors (CARs), and recombinant immune cells comprising such expression constructs (e.g., CAR T-cells and CAR T-regs). In some embodiments, CAR T-cells described herein are useful for killing immune cells (e.g., APCs), which APCs induce immune responses against AAV capsid proteins, thereby dampening (e.g., inhibiting or suppressing) the host immune response against the AAV capsid protein. In some embodiments, CAR T-reg cells described herein are useful for inhibiting the killing of immune cells (e.g., APCs), which APCs induce immune responses against AAV capsid proteins, thereby increasing (e.g., promoting or inducing) the host immune response against AAV capsid protein.

### Chimeric Antigen Receptors (CARs)

Aspects of the disclosure relate to compositions (e.g., isolated nucleic acids, vectors such as viral vectors, cells, etc.) encoding one or more chimeric antigen receptors (CARs). For example, in aspects, the disclosure relates to CARs having an antigen binding domain that is specific for an AAV capsid protein epitope. As used herein, a “chimeric antigen receptor” or “CAR” refers to a recombinant cell receptors which redirects the specificity or function of a cell (e.g., an immune cell) by providing both antigen-binding and cell activating functions. Generally, CARs are a fusion protein comprising one or more antigen binding domains (e.g., extracellular domain), a transmembrane domain, and at least one cytoplasmic signaling domain (e.g., intracellular domain), which combination of extracellular domain, transmembrane domain, and cytoplasmic signaling domain, do not naturally occur together in nature. A cell expressing a CAR may produce, in some embodiments, an atypical cellular response (e.g., increased, decreased, or different cellular response relative to a naturally-occurring cell).

### Antigen Binding Domain

A CAR may comprise one or more (e.g., 1, 2, 3, etc.) antigen binding domains (e.g., an extracellular domain). As used herein, an “antigen binding domain,” refers to the domain of a protein or polypeptide external to the cellular membrane, which domain's main function is to recognize (e.g., bind) and respond to a type of ligand (e.g., antigen). As used herein, “binding affinity” refers to the apparent association constant or KA. The KA is the reciprocal of the dissociation constant (KD). The antigen binding domain (e.g., antibody, scFv, etc.) described herein may have a binding affinity (KA) of at least 10⁵, 10⁶, 10⁷, 10⁸, 10⁹, 10¹⁰ M, or higher. An increased binding affinity corresponds to a decreased KD. Higher affinity binding of an antigen binding domain to a first target relative to a second target can be indicated by a higher KA (or a smaller numerical value KD) for binding the first target than the KA (or numerical value KD) for binding the second target. In such cases, the antigen binding domain has specificity for the first target relative to the second target. Differences in binding affinity between the first target relative to the second target (e.g., for specificity or other comparisons) can be greater than 1, for example at least 1.5; 2; 3; 4; 5; 10; 15; 20; 37.5; 50; 70; 80; 91; 100; 500; 1,000; 10,000, or 100,000 fold.

Binding affinity can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in, e.g., TRIS-buffer (50 mM TRIS, 150 mM NaCl, 5 mM CaCl2) at pH7.5). These techniques can be used to measure the concentration of bound binding protein as a function of target protein concentration. The concentration of bound binding protein ([Bound]) is related to the concentration of free target protein ([Free]) and the concentration of binding sites for the binding protein on the target where (N) is the number of binding sites per target molecule by the following equation: [Bound]=[N][Free]/(Kd+[Free]).

It is not always necessary to make an exact determination of KA, though, since sometimes it is sufficient to obtain a quantitative measurement of affinity, e.g., determined using a method such as ELISA or FACS analysis, is proportional to KA, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

Each of the antigen binding domains may bind to the same target (e.g., the same peptide, protein, epitope, etc.), or each antigen binding domain may bind to a separate target (e.g., a different peptide, protein, epitope, etc., for example as in the case of a bi-specific CAR).

An antigen binding domain, may be part of a polypeptide which crosses the cellular membrane multiple times, which results in a multiple exposures of the polypeptide with loops intersecting the membrane as well as an end with a singular intersection. In some embodiments, extracellular ligand-binding domain or moiety is in the form of a binding protein, small molecule, a peptide, a targeting agent, a protein agonist, or a protein antagonist.

The antigen binding domain can be any domain that binds to the antigen including, but not limited to, monoclonal antibodies, scFvs, polyclonal antibodies, synthetic antibodies, human antibodies, humanized antibodies, and fragments thereof. In some instances, it is beneficial for the antigen binding domain to be derived from the same species in which the CAR will ultimately be used. For example, for use in humans, it may be beneficial for the antigen binding domain of the CAR to comprise a human antibody or fragment thereof. In some embodiments, the antigen binding domain comprises a human antibody or a fragment thereof. In some embodiments, the binding protein is an antibody, an antigen-binding portion of an antibody (e.g., a scFv), a ligand, a cytokine, or a receptor. In some embodiments, an antigen binding domain may comprise a site derived from a monoclonal antibody or a scFv. In some embodiments, the antigen binding fragment is a scFv or an Fab fragment. In some embodiments, the antigen binding domain may bind an AAV capsid protein epitope. In some embodiments, an scFv fragment is encoded by a nucleic acid sequence comprising a sequence with at least 70% identity (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to the sequence of SEQ ID NO: 22. In some embodiments, an scFv fragment is encoded by a nucleic acid sequence comprising a sequence of SEQ ID NO: 22. In some embodiments the chimeric antigen receptors of the disclosure comprise a CD19 extracellular domain. In some embodiments, the CD19 extracellular domain is encoded by a nucleic acid sequence comprising a sequence with at least 70% identity (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to the sequence of SEQ ID NO: 32. In some embodiments, the CD19 extracellular domain is encoded by a nucleic acid sequence comprising a sequence of SEQ ID NO: 32. In some embodiments the chimeric antigen receptors of the disclosure comprise an EGFR extracellular domain. In some embodiments, the EGFR extracellular domain is encoded by a nucleic acid sequence comprising a sequence with at least 70% identity (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to the sequence of SEQ ID NO: 33. In some embodiments, the EGFR extracellular domain is encoded by a nucleic acid sequence comprising a sequence of SEQ ID NO: 33.

In some embodiments, a CAR may be configured to have an antigen binding domain which recognizes an epitope of a capsid protein of an AAV. Each serotype of AAV has a capsid comprising 3 capsid proteins (e.g. VP1, VP2, VP3), and which capsid expresses a variety of receptors. Of the AAV serotypes the capsid proteins contain 12 hypervariable regions which capsid proteins affect the tissue specificity of the AAV. The serotypes and capsids also express a number of stimulating epitopes. In some embodiments, the nucleic acid encodes a CAR with an antigen binding domain targets (e.g., specifically binds to) an epitope of an AAV capsid protein. In some embodiments, the nucleic acid encodes a CAR with an antigen binding domain targets (e.g., specifically binds to) an epitope of an AAV capsid protein having a serotype selected from AAV1, AAV2, AAV3, AAV3b, AAV4, AAV5, AAV6, AAV7, AAV8, AAVrh.8, AAV9, AAV10, AAVrh.10, AAVrh.39, AAVrh.43, AAV.PHPB, AAV.PHPB.e, AAVrh32.33, or a variant thereof. In some embodiments, the nucleic acid encodes a CAR with an antigen binding domain which targets an AAV2 epitope or an AAV6 epitope.

In some embodiments, the antigen binding domain targets an epitope of an AAV capsid protein having a serotype selected from AAV1, AAV2, AAV3, AAV3b, AAV4, AAV5, AAV6, AAV7, AAV8, AAVrh.8, AAV9, AAV10, AAVrh.10, AAVrh.39, AAVrh.43, AAV.PHPB, AAV.PHPB.e, AAVrh32.33, or a variant thereof. In some embodiments, the nucleic acid may encode an antigen binding domain which targets an epitope of a capsid protein of any of AAV1, AAV2, AAV3, AAV3b, AAV4, AAV5, AAV6, AAV7, AAV8, AAVrh.8, AAV9, AAV10, AAVrh.10, AAVrh.39, AAVrh.43, AAV.PHPB, AAV.PHPB.e, AAVrh32.33, or a variant thereof, AAV serotypes. In some embodiments, the antigen binding domain targets an AAV2 epitope or an AAV6 epitope. In some of the embodiments, nucleic acid may encode an antigen binding domain which targets an epitope of a capsid protein of either AAV2 or AAV6, AAV serotypes. In some of the embodiments, the antigen binding domain targets an epitope of a capsid protein of AAV serotype AAV2. In some embodiments, the antigen binding domain has a nucleic acid comprising SEQ ID NO: 22. In some embodiments, the nucleic acid encodes an antigen binding domain having an amino acid comprising SEQ ID NO: 39. In some of the embodiments, the antigen binding domain targets an epitope of a capsid protein of AAV serotype AAV6.

The antigen binding domain may comprise an scFv having a variable heavy chain domain (VH) derived from an AAV capsid protein-targeting antibody linked to a variable light chain domain (VL) derived from an AAV capsid protein-targeting antibody. In some embodiments, the nucleic acid encodes a CAR with an antigen binding domain which comprises a scFv. Single chain variable fragments are well known in the art, they are generally known to consist of a fusion protein of the VH and VL of the subject antibody connected by a linker peptide.

In some embodiments, a CAR may have increased cytotoxic effects on a cell bearing a target antigen or epitope. In some embodiments, a CAR may have increased cytotoxic effects on a cell bearing a target antigen or epitope resulting from expressing a target antigen binding domain on a T-Cell to target APCs. In some embodiments, a CAR may have decreased cytotoxic effects on a cell bearing a target antigen or epitope. In some embodiments, a CAR may have decreased cytotoxic effects on a cell bearing a target antigen or epitope resulting from expressing a target antigen binding domain on a T-reg cell to induce APCs.

In some embodiments, a nucleic acid encoding a CAR is disclosed, wherein the antigen binding domain comprises a first portion comprising a monoclonal antibody or a single chain variable fragment (scFv). In some embodiments, the nucleic acid encodes a CAR with an antigen binding domain comprising a “monoclonal antibody” (mAb). In some embodiments, the antigen binding domain may be a mAb. In some embodiments, the mAb may be an mAb to, or targets, an epitope of AAV capsid. In some embodiments, the mAb may be an mAb to, or targets, an epitope of AAV1, AAV2, AAV3, AAV3b, AAV4, AAV5, AAV6, AAV7, AAV8, AAVrh.8, AAV9, AAV10, AAVrh.10, AAVrh.39, AAVrh.43, AAV.PHPB, AAV.PHPB.e, AAVrh32.33, or a variant thereof. In some embodiments, the mAb may be an mAb, or targets, AAV2 capsid epitope or an AAV6 capsid epitope. mAbs are antibodies that are made by immune cells (e.g., leukocytes) that are identical and bind to a shared epitope. Antibodies (e.g., immunoglobulins) are well known in the art, but generally are known to be large “Y” shaped proteins in the immune system which neutralize antigens. Each protein consist of two identical heavy chains and two identical light chains each having a variable (e.g., variable domain of heavy chain (VH) and variable domain of light chain (VL)). The variable domains contain complementarity determining regions, which specify the antigen the antibody will recognize (i.e. bind). In addition to the variable domains, the light chain a constant domain (CL) and the heavy chain has three constant domains, which are numbered 1-3 as you move farther from the VH domain (e.g., CH1, CH2, CH3). Production of mAbs in all forms (e.g., humanized) are well known in the art and include traditional means such as animal based techniques. For example, the techniques generally entail: 1) exposing a subject (e.g., mouse, rabbit) to an antigen (e.g., capsid protein); 2) fusion into immortalized cell lines (e.g., myeloma cells); 3) cell culture and antibody screening; and 4) selection and recovery of cells presenting the mAb of interest) as well as other techniques such as phage display (generally: 1) the target proteins or nucleic acid sequences (e.g., capsid proteins) are immobilized to the wells of a microtiter plate; 2) a variety of nucleic acid sequences are expressed in a bacteriophage library in the form of fusions with the bacteriophage coat protein, so that they are displayed on the surface of the viral particle. The protein displayed corresponds to the genetic sequence within the phage; 3) this phage-display library is added to the microtiter plat allowing the phage time to bind, the dish is subsequently washed; 4) bound phage-displaying proteins remain attached to the dish, while all others are washed away; 5) attached phage may be enriched (through elution and culture, etc.) and steps 3 to 5 are optionally repeated one or more times, further enriching the phage library in binding proteins; 6) following further bacterial-based culture, the nucleic acids within in the interacting phage is sequenced to identify the interacting proteins or protein fragments). Any method of generating mAbs known in the art may be used to generate the mAbs of the instant disclosure.

In some embodiments, the nucleic acid of the instant disclosure encoding the antigen binding domain may further encode an immunoglobulin G1 (IgG1) constant domain 2 (IgG1 CH2). In some embodiments, the nucleic acid of the instant disclosure, may further encode for an IgG1 constant domain 3 (IgG1 CH3). IgG is a type of antibody which represents, by some estimates, nearly 75% of the serum antibodies in humans. IgG is created by B-cells and has two antigen binding sites. IgG is a versatile immunoglobulin, exploiting a variety of mechanisms to mediate the immune response, for example through classical pathways, toxin neutralization, complement pathways, opsonization, agglutination, antibody dependent cell-mediated cytotoxicity, and others. IgG is further differentiated into 4 subclasses (G1, G2, G3, and G4). The structure of the hinge region which links the CH1 domain with the CH2 domain, determines the properties and subclass of IgG molecules. The hinge region also affects the affinity of IgG to immunoglobulin receptor (e.g. Fc receptor) on a variety of immune cells, thereby affecting the specificity and activity of the sub-class IgG.

In some embodiments, the antigen binding domain may further comprise an extracellular recognition domain. A recognition domain may be used to identify the cells upon action or inaction when the protein is used in vitro or in vivo, for example by release of visible light, or by being detectable by different modalities (e.g., x-ray, luminescence, radiation, or other detectable means). Such domains (e.g., tags) are known in the art and will be readily appreciated by the skilled artisan (e.g., GFP, EGFP, etc.). In some embodiments, the antigen binding domain further comprises EGFP.

### Transmembrane Domain

As used herein, a “transmembrane domain,” refers to the domain of a protein or polypeptide which spans the cellular membrane connecting the antigen binding domain with the cytoplasmic signaling domain. The transmembrane domain may span the cellular membrane multiple times and is responsible for communicating the activation of the antigen binding domain (e.g., through binding of a ligand or antigen) with the cytoplasmic signaling domain. Any transmembrane domain is contemplated for use herein as long as the domain is capable of anchoring a CAR comprising the domain to a cell membrane. In some embodiments, the transmembrane domain that naturally is associated with one of the domains in the CAR is used. In some instances, the transmembrane domain can be selected or modified by amino acid substitution to avoid binding of such domains to the transmembrane domains of the same or different surface membrane proteins to minimize interactions with other members of the receptor complex. Various mechanisms exist for signaling, such as conformational changes and changes to the cellular membrane (e.g., conformational or changes to membrane pores), and are known in the art.

The transmembrane domain may be selected from any known transmembrane domain which can be incorporated into the nucleic acid and expressed as a fusion protein. Transmembrane domains of particular use in this invention may be derived from (e.g., comprise at least the transmembrane domain(s) of) the alpha, beta or zeta chain of the T-cell receptor, CD28, CD3 epsilon, CD45, CD4, CD5, CD8, CD9, CD16, CD22, CD33, CD37, CD64, CD80, CD86, C 5 D134, CD137, and CD154. Transmembrane domains can be identified using any method known in the art or described herein, e.g., by using the UniProt Database.

In some embodiments, the transmembrane domain is a CD28 transmembrane domain. CD28 is a costimulatory receptor for T-cells which is primarily responsible for delivering a second signal for T-cell activation. In some embodiments, the nucleic acid encoding a transmembrane domain has a nucleic acid sequence comprising a sequence with at least 70% identity (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to SEQ ID NO: 25. The terms “percent identity,” “sequence identity,” “% identity,” “% sequence identity,” and % identical,” as they may be interchangeably used herein, refer to a quantitative measurement of the similarity between two sequences (e.g., nucleic acid or amino acid). The percent identity of genomic DNA sequence, intron and exon sequence, and amino acid sequence between humans and other species varies by species type, with chimpanzee having the highest percent identity with humans of all species in each category. Percent identity can be determined using the algorithms of Karlin and Altschul, Proc. Natl. Acad. Sci. USA 87:2264-68, 1990, modified as in Karlin and Altschul, Proc. Natl. Acad. Sci. USA 90:5873-77, 1993. Such algorithms is incorporated into the NBLAST and XBLAST programs (version 2.0) of Altschul et al., J. Mol. Biol. 215:403-10, 1990. BLAST protein searches can be performed with the XBLAST program, score=50, word length=3, to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used. When a percent identity is stated, or a range thereof (e.g., at least, more than, etc.), unless otherwise specified, the endpoints shall be inclusive and the range (e.g., at least 70% identity) shall include all ranges within the cited range (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) and all increments thereof (e.g., tenths of a percent (i.e., 0.1%), hundredths of a percent (i.e., 0.01%), etc.). In some embodiments, the transmembrane domain is encoded by a nucleic acid sequence comprising the sequence of SEQ ID NO: 25. In some embodiments, the antigen binding domain and the transmembrane domain are connected (e.g., joined) by a linker.

### Cytoplasmic Signaling Domain

In some embodiments, a CAR comprises a cytoplasmic signaling domain. As used herein, “cytoplasmic signaling domain” (e.g., intracellular domain) refers to one or more domains (e.g., co-stimulatory domains, signaling domains) of a protein or polypeptide internal to the cellular membrane, which domain's main function is to relay the signal from the antigen binding and transmembrane domains to interact with the interior of the cell. The interaction can be by various mechanisms (e.g., protein-protein interactions, enzymatic activity) and are known in the art. The cytoplasmic signaling domain may be part of a polypeptide which crosses the cellular membrane multiple times, which results in a multiple exposures of the polypeptide with loops intersecting the membrane as well as an end with a singular intersection. Cytoplasmic signaling domains transmit an activation signal to the cell following binding of the antigen binding domain (i.e., extracellular domain). Cytoplasmic signaling domain can be any intracellular signaling domain of interest that is known in the art. For example, cytoplasmic signaling domains can include, without limitation, CD3-zeta. This signaling in turn “activates” or causes a function of the cell (i.e., effector function). Additionally, while an entire intracellular signaling domain (i.e., not truncated) can be employed, in cases it may not be necessary to use the entire domain, for example a truncated portion of the intracellular signaling domain may function as the intracellular domain with equivalent, or modulated, function. Such truncated portion may be used in place of the intact domain as long as it transduces the effector function signal. The term intracellular signaling domain is thus meant to include any truncated portion of the intracellular signaling domain sufficient to transduce the effector function signal.

In some embodiments, the cytoplasmic signaling domain comprises domain selected from: a CD28 signaling domain, a 4-1BB signaling domain, a CD3 signaling domain, or combination thereof. In some embodiments, the cytoplasmic signaling domain comprises a CD28 signaling domains. In some embodiments, the cytoplasmic signaling domain comprises a 4-1BB signaling domains. In some embodiments, the cytoplasmic signaling domain comprises a CD3 signaling domains. In some embodiments, the cytoplasmic signaling domain comprises all three of CD28, 4-1BB, and CD3 signaling domains.

The cytoplasmic signaling domain of CD28 is a B7 (e.g., CD80 and CD 86) receptor constitutively expressed on naive T-cells and can initiate a variety of pathways to stimulate a T-cell response. The cytoplasmic signaling domain of 4-1BB is a co-stimulatory molecule with roles in expansion, acquisition of effector function, survival, and development of T cell memory. It is a signaling domain with a role in pathways which induce the expression of survival genes encoding surviving, Bcl-2, Bcl-XL, and Bfl-1 and decrease the expression of pro-apoptotic Bim, which can promote different type cells to live. CD3 is a T-cell co-receptor integral to T-cell activation. CD3 molecules, along with the t-cell receptor, associate to comprise the t-cell receptor complex, which activation is necessary for native T-cell activation. In some embodiments, the nucleic acid may encode a CAR having a cytoplasmic signaling domain which is a CD28 signaling domain. In some embodiments, the CD28 signaling domain is encoded by a nucleic acid sequence comprising a sequence with at least 70% identity (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to the sequence of SEQ ID NO: 26. In some embodiments, the CD28 signaling domain is encoded by a nucleic acid sequence comprising a sequence of SEQ ID NO: 26. In some embodiments, the CD28 signaling domain is encoded by a nucleic acid sequence comprising a sequence with at least 70% identity (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to the sequence of SEQ ID NO: 27. In some embodiments, the CD28 signaling domain is encoded by a nucleic acid sequence comprising a sequence of SEQ ID NO: 27. In some embodiments, the nucleic acid may encode a CAR having a cytoplasmic signaling domain which is a 4-1BB signaling domain. In some embodiments, the CD28 signaling domain is encoded by a nucleic acid sequence comprising a sequence with at least 70% identity (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to the sequence of SEQ ID NO: 28. In some embodiments, the CD28 signaling domain is encoded by a nucleic acid sequence comprising a sequence of SEQ ID NO: 28. In some embodiments, the CD28 signaling domain is encoded by a nucleic acid sequence comprising a sequence with at least 70% identity (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to the sequence of SEQ ID NO: 29. In some embodiments, the CD28 signaling domain is encoded by a nucleic acid sequence comprising a sequence of SEQ ID NO: 29. In some embodiments, the nucleic acid may encode a cytoplasmic signaling domain which is a CD3 signaling domain. In some embodiments, the CD3 signaling domain is a CD3z signaling domain. In some embodiments, the CD3 signaling domain is encoded by a nucleic acid sequence comprising a sequence with at least 70% identity (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to the sequence of SEQ ID NO: 30. In some embodiments, the CD3 signaling domain is encoded by a nucleic acid sequence comprising a sequence of SEQ ID NO: 30. In some embodiments, the CD28 signaling domain is encoded by a nucleic acid sequence comprising a sequence with at least 70% identity (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to the sequence of SEQ ID NO: 31. In some embodiments, the CD28 signaling domain is encoded by a nucleic acid sequence comprising a sequence of SEQ ID NO: 31.

The three domains, antigen binding domains, transmembrane domains, and cytoplasmic signaling domains, as well as the general function and architecture are known in the art. The three domains (e.g., antigen binding domain, transmembrane domain, and cytoplasmic signaling domain), operate to effect some change in the host cell, for example increased cellular activity (e.g. protein production), which can result in various internal or external effects (e.g. cytotoxic effect on antigen bearing cell).

### Linkers

In some embodiments, the disclosure relates to a CAR comprising a linker. In some embodiments, domains of the CAR may be joined by a linker. In some embodiments, at least one linker is used to join each domain of a CAR. The term “linker,” as used herein, refers to a molecule linking two other molecules or moieties. Linkers are well known in the art and can comprise any suitable combination of nucleic acids or amino acids to facilitate the proper function of the structures they join. The linker can be a series of amino acids. The linker can be an amino acid sequence in the case of a linker joining two fusion proteins. For example, a fusion protein (e.g., a CAR) comprising various domains (e.g., antigen binding domain, transmembrane domain, intracellular domain) can be fused to by an amino acid linker sequence. The linker can also be a nucleotide sequence in the case of joining two nucleotide sequences together. In other embodiments, the linker is an organic molecule, group, polymer, or chemical moiety. In some embodiments, the linker is 1-100 amino acids in length, for example: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 30-35; 35-40; 40-45; 45-50; 50-60; 60-70; 70-80; 80-90; 90-100; 100-150; or 150-200 amino acids in length. In some embodiments, the linker is 5-1,000 nucleotides in length, for example: 5; 6; 7; 8; 9; 10; 11; 12; 13; 14; 15; 16; 17; 18; 19; 20; 21; 22; 23; 24; 25; 26; 27; 28; 29; 30; 30-35; 35-40; 40-45; 45-50; 50-60; 60-70; 70-80; 80-90; 90-100; 100-150; 150-200; 200-300; 300-500; 500-1,000; 1,000-2,000; or 2,000-5,000 nucleotides. Longer or shorter linkers are also contemplated. In some embodiments, a CAR comprises at least one linker. In some embodiments, a CAR comprises at least one linker. In some embodiments, a CAR comprises at least two linkers. In some embodiments, a CAR comprises at least three linkers. In some embodiments, a CAR comprises at least four linkers. In some embodiments, a CAR comprises at least five linkers. In some embodiments, a CAR comprises more than five linkers. In some embodiments, all of the linkers used are the same (e.g., identical). In some embodiments, not all of the linkers are identical (e.g., at least one linker is distinct from at least one other linker). In some embodiments, each linker is distinct from each other linker.

In some embodiments, a linker is referred to as a hinge or hinge domain. As used herein, a hinge domain generally means any polypeptide that functions to provide flexibility to the CAR, or domains thereof, and/or prevent steric hindrance of the CAR, or domains thereof. In some embodiments, a hinge domain may comprise up to 300 amino acids, preferably 10 to 100 amino acids and most preferably 1 to 20 amino acids. It also should be appreciated that one or more hinge domains may be included in other regions of a CAR, as aspects of the disclosure are not limited in this respect. In some embodiments, the hinge is encoded by a nucleic acid sequence having at least 70% identity (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to the sequence of SEQ ID NO: 23. In some embodiments, the hinge is encoded by a nucleic acid sequence comprising a sequence of SEQ ID NO: 23. In some embodiments, the hinge is encoded by a nucleic acid sequence having at least 70% identity (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to the sequence of SEQ ID NO: 24. In some embodiments, the hinge is encoded by a nucleic acid sequence comprising a sequence of SEQ ID NO: 24.

In some embodiments the VH and the VL are linked by a linking molecule. In some embodiments, the linking molecule is a peptide linker. The linkers may be rich in glycine (e.g., a (GGGGS)n linker, where n is an integer between 1 and 10) for flexibility and in serine or threonine for solubility (i.e., hinge). In some embodiments, the peptide linker is a glycine-rich linker. The linker may connect the N-terminus of either VH or VL to the C-terminus of the other. In some embodiments, the linker joins the N-terminus of the VH to the C-terminus of the VL. In some embodiments, the linker joins the N-terminus of the VL to the C-terminus of the VH. In some embodiments, an antigen binding domain may be an scFv having a VH derived from an AAV capsid protein-targeting antibody linked to a VL derived from an AAV capsid protein-targeting antibody.

In some embodiments, the nucleic acid comprises IgG CH2 and IgG1 CH3 domains are joined by a linker. In some embodiments, the linker comprises an IgG1 hinge linker and/or an IgG2 hinge linker. In some embodiments, the nucleic acid of the instant disclosure encoding the antigen binding domain may further encode for an immunoglobulin G1 (IgG1) constant domain 2 (IgG1 CH2) and constant domain 3 (IgG1 CH3) joined by a linker. In some embodiments, the linker comprises a linker native to IgG1. In some embodiments, the nucleic acid of the instant disclosure encoding the antigen binding domain may further encode for an immunoglobulin G1 (IgG1) constant domain 2 (IgG1 CH2) and constant domain 3 (IgG1 CH3) linked to the first portion by a hinge linker native to IgG1.

In some embodiments, a CAR is encoded by a nucleic acid sequence having at least 70% identity (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to the sequence of SEQ ID NO: 1-4 and 16-17. In some embodiments, a CAR is encoded by a nucleic acid comprising a sequence of SEQ ID NO: 1-4 and 16-17.

### Isolated Nucleic Acids

In some embodiments, a CAR is encoded by an isolated nucleic acid. As used herein, the term “isolated,” refers to the characteristic of a material as provided herein being removed from its original or native environment (e.g., the natural environment if it is naturally occurring). Therefore, a naturally-occurring polynucleotide or protein or polypeptide present in a living animal is not isolated, but the same polynucleotide or polypeptide, separated by human intervention from some or all of the coexisting materials in the natural system, is isolated. An artificial or engineered material, for example, a non-naturally occurring nucleic acid construct, such as the expression constructs and vectors described herein, are, accordingly, also referred to as isolated. A material does not have to be purified in order to be isolated. Accordingly, a material may be part of a vector and/or part of a composition, and still be isolated in that such vector or composition is not part of the environment in which the material is found in nature.

As used herein, the term “nucleic acid,” refers to a polymer of nucleotides. The term includes, but is not limited to, oligonucleotides and polynucleotides, both single-stranded and double-stranded forms, including hybrids, for example, of DNA and RNA strands, or of strands comprising ribonucleotides, deoxyribonucleotides, and/or modified nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5 bromouridine, C5 fluorouridine, C5 iodouridine, C5 propynyl uridine, C5 propynyl cytidine, C5 methylcytidine, 7 deazaadenosine, 7 deazaguanosine, 8 oxoadenosine, 8 oxoguanosine, O(6) methylguanine, 4-acetylcytidine, 5-(carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′ N phosphoramidite linkages).

In some embodiments, the isolated nucleic acid encoding a CAR may be a portion of an expression construct. The term “expression construct,” as used herein, refers to a nucleic acid construct comprising nucleic elements sufficient for the expression of a gene product. Typically, an expression construct comprises a nucleic acid encoding a gene product operatively linked to a promoter sequence. The term “operatively linked” refers to the association of two or more nucleic acid fragments on a single nucleic acid fragment so that the function of one is affected by the other. For example, a promoter is operatively linked with a coding sequence when it is capable of affecting the expression of that coding sequence (e.g., the coding sequence is under the transcriptional control of the promoter). Encoding sequences can be operatively linked to regulatory sequences in sense or antisense orientation. In some embodiments, the promoter is a heterologous promoter.

The term “promoter,” as used herein, refers to a nucleotide sequence capable of controlling the expression of a coding sequence or functional nucleic acid. In general, a nucleic acid sequence encoding a gene product is located 3′ of a promoter sequence. In some embodiments, a promoter sequence consists of proximal and more distal upstream elements and can comprise an enhancer element. An “enhancer” is a nucleotide sequence that can stimulate promoter activity and may be an innate element of the promoter or a heterologous element inserted to enhance the level or tissue-specificity of a promoter. In some embodiments, the promoter is derived in its entirety from a native gene. In some embodiments, the promoter is composed of different elements derived from different naturally occurring promoters. In some embodiments, the promoter comprises a synthetic nucleotide sequence. It will be understood by those skilled in the art that different promoters will direct the expression of a gene in different tissues or cell types, or at different stages of development, or in response to different environmental conditions or to the presence or the absence of a drug or transcriptional co-factor.

Ubiquitous, cell-type-specific, tissue-specific, developmental stage-specific, and conditional promoters, for example, drug-responsive promoters (e.g., tetracycline-responsive promoters) are well known to those of skill in the art. In some embodiments, the promoter is a RNA polymerase I promoter. In some embodiments, the promoter is a RNA polymerase II promoter. In some embodiments, the promoter is a RNA polymerase III promoter. Promoters mediating transcription by recruiting RNA polymerase I (e.g., most rRNA gene promoters), II (e.g., U6 and H1 promoters), or III (e.g., most promoters of protein-encoding genes), are well known to those of skill in the art. The term “heterologous promoter,” as used herein, refers to a promoter that is not found to be operatively linked to a given encoding sequence in nature. In some embodiments, an expression construct may comprise additional elements, for example, an intron, an enhancer, a polyadenylation site, a WPRE, and/or other elements known to affect expression levels of the encoding sequence. Without wishing to be bound by theory, inclusion of an intron in an expression construct, for example, between the transcriptional start site and an encoding nucleic acid sequence, for example, a protein-encoding cDNA sequence, is believed to result in increased expression levels of the encoding nucleic acid and the encoded gene product as compared to an expression construct not including an intron. In some embodiments, the promoter may be an EF1-alpha promoter. In some embodiments, the EF1-alpha promoter comprises a nucleic acid sequence having at least 70% identity (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to the sequence of SEQ ID NO: 5 or 19. In some embodiments, the EF1-alpha promoter comprises a nucleic acid sequence comprising a sequence of SEQ ID NO: 5 or 19. In some embodiments, the promoter may be a cytomegalovirus (CMV) promoter. In some embodiments, the CMV promoter comprises a nucleic acid sequence having at least 70% identity (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to the sequence of SEQ ID NO: 19. In some embodiments, the CMV promoter comprises a nucleic acid sequence comprising a sequence of SEQ ID NO: 19. In some embodiments, the promoter may be a MND2 promoter. In some embodiments, the MND2 promoter comprises a nucleic acid sequence having at least 70% identity (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to the sequence of SEQ ID NO: 20. In some embodiments, the MND2 promoter comprises a nucleic acid sequence comprising a sequence of SEQ ID NO: 20. In some embodiments, the promoter may be a SFFV promoter. In some embodiments, the SFFV promoter comprises a nucleic acid sequence having at least 70% identity (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to the sequence of SEQ ID NO: 21. In some embodiments, the SFFV promoter comprises a nucleic acid sequence comprising a sequence of SEQ ID NO: 21.

The term “gene product,” as used herein, refers to any product encoded by a nucleic acid sequence. Accordingly, a gene product may, for example, be a primary transcript, a mature transcript, a processed transcript, or a protein or peptide encoded by a transcript. Examples for gene products, accordingly, include mRNAs, rRNAs, tRNAs, hairpin RNAs, microRNAs (miRNAs), shRNAs, siRNAs, and peptides and proteins, for example, reporter proteins or therapeutic proteins. In some embodiments, the expression construct further comprises a promoter operably linked to a nucleic acid sequence encoding the CAR. The term “operably linked,” as may be used herein, refers to functional linkage between a regulatory sequence and a heterologous nucleic acid sequence (e.g., transgene) resulting in expression of the heterologous nucleic acid sequence (e.g., transgene). For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked nucleic acid sequences are contiguous and, where necessary to join two protein coding regions, in the same reading frame.

In addition to the conventional control elements necessary, the nucleic acids may also include elements which are operably linked to the transgene in a manner which permits its transcription, translation and/or expression in a cell transfected with the plasmid vector or infected with the virus produced by the invention. In some embodiments, a promotor is contiguous with the gene of interest and expression control sequences that act in trans or at a distance to control the transgene. Expression control sequences include appropriate transcription initiation, termination, promoter, and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product (e.g., enhancers, post-transcriptional regulatory elements). In some embodiments, the polyA signal is an SV40 polyA signal. A great number of expression control sequences, including promoters which are native, constitutive, inducible, and/or tissue-specific, are known in the art and may be utilized. Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV 40 promoter, the dihydrofolate reductase promoter, the beta-actin promoter, the phosphoglycerol kinase (PGK) promoter, and the EF1alpha promoter [Invitrogen]. In some embodiments, EF1alpha has a sequence with at least 70% identity (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to the sequence of SEQ ID NO: 5. In some embodiments, EF1alpha has a sequence of SEQ ID NO: 5. In some embodiments, the nucleic acid is has a promoter operably linked to the nucleic acid sequence encoding a CAR.

In some embodiments, the promoter comprises an EF1alpha promoter. EF1alpha is a constitutive promotor which is especially useful in instances where other promoter sequences have diminished activity, or where other promoters may experience silencing or interferences from other cellular responses or activities. It is a common promoter used in driving ectopic gene expression in vivo. In some embodiments, the EF1alpha promoter may be operably linked to the transgene.

In some embodiments, the nucleic acids comprise a post-transcriptional regulatory element (PRE). Post-transcriptional regulatory elements, control gene expression through a variety of mechanisms (e.g., capping, splicing, polyadenylation, editing, stability manipulation, binding), but generally effect control at the RNA level, thus influence gene expression post-transcription, but pre-translation. PRE's may be cis- or trans-acting, and may be operably linked to the transgene. An example of a PRE, is woodchuck hepatitis virus PRE (WPRE), which is a nucleic acid sequence that in its transcribed form creates a tertiary structure which enhances gene expression. It's comprised of three component elements, alpha, beta, and gamma, which alpha being able to effect enhancement by itself, but in a reduced capacity. In some embodiments, the nucleic acid may encode a PRE. In some embodiments, the PRE comprises a Woodchuck Hepatitis Virus Posttranscriptional Regulatory Element (WPRE). In some embodiments, the WPRE may have an alpha component comprising a nucleic acid sequence with at least 70% identity (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to SEQ ID NO: 6. In some embodiments, the WPRE may have an alpha component with a nucleic acid sequence comprising SEQ ID NO: 6. In some embodiments, the nucleic acid may comprise a WPRE having an alpha component comprising an amino acid sequence of SEQ ID NO: 6. In some embodiments, the nucleic acid comprises a WPRE with at least 70% identity to SEQ ID NO: 7. In some embodiments, the WPRE has a nucleic acid sequence comprising SEQ ID NO: 7. In some embodiments, there is more than one PRE encoded in the nucleic acid, and the more than one PRE can include WPRE, or a mixture thereof. In some embodiments, the nucleic acid does not include a PRE.

In some embodiments, the EF1alpha promoter is positioned 5′ to a nucleic acid sequence encoding the CAR, and the WPRE is positioned 3′ to the nucleic acid sequence encoding the CAR. Either the EF1alpha promoter or the WPRE may be positioned anywhere such that they effectuate transcription of the CAR. In some embodiments, the EF1alpha promoter may be positioned 5′ to the nucleic acid encoding the CAR. In some embodiments, the WPRE may be positioned 3′ to the nucleic acid encoding the CAR. The transgene can be positioned anywhere within the nucleic acid which to effectuate its expression (e.g., operably linked to its regulatory elements (e.g., promoter)). In instances where the transgene isn't protein coding, it may be positioned in any untranslated portion of the nucleic acid, including, for example, an intron, a 5′ or 3′ untranslated region, etc.

In some embodiments, the nucleic acid may encode genes and/or gene products useful in modulating the immune response. For example, forkhead box P3 (FoxP3) is a gene involved in regulating immune system responses. The protein produced by FoxP3 has been observed to be a regulator of the pathway of the development and function of T-regs. In some embodiments, the nucleic acid described herein, may further comprise a nucleic acid sequence encoding FoxP3. In some embodiments, the FoxP3 encoding nucleic acid sequence is linked to the nucleic acid sequence encoding the CAR. In some embodiments, the FoxP3 encoding nucleic acid sequence is linked to the nucleic acid sequence encoding the CAR, wherein the sequences are linked by a nucleic acid sequence encoding a 2A self-cleaving peptide. 2A self-cleaving peptides are generally 18-22 amino acids long and induce cleaving of a recombinant protein between the proline and glycine in the C-terminus of the 2A peptide. The 2A peptide sequence allows for the recombinant protein to be severed in to two smaller proteins and function independent of the other and free from any effects experienced by the larger fusion protein. Four variants of the 2A sequence are commonly used; T2A (SEQ ID NO: 8 and 35), P2A (SEQ ID NO: 9 and 36), E2A (SEQ ID NO: 10 and 34), and F2A (SEQ ID NO: 11). In some embodiments, the 2A peptide linker is selected from T2A, P2A, E2A, and F2A. In some embodiments, the 2A peptide linker is T2A. In some embodiments, the 2A peptide linker is P2A. In some embodiments, the 2A peptide linker is E2A. In some embodiments, the 2A peptide linker is F2A. In some embodiments, the 2A peptide linker is selected from SEQ ID NOs: 8-11 and 34-36. In some embodiments, the 2A linker peptide includes a GSG sequence (glycine (G)-serine (S)-glycine (G)) on the N-terminus, which may increase cleavage efficiency and is selected from SEQ ID NOs: 12-15 or GSG added to SEQ ID NO: 34-36.

In some embodiments, the nucleic acid encoding FoxP3 may be linked to the nucleic acid encoding the CAR. However, in some instances, it may be beneficial to have FoxP3 protein and CAR accessible in vivo as separate proteins. This may be accomplished by encoding the genes as separable expression constructs with the necessary additional transcriptional factors (e.g. promoters and others as described herein). In some embodiments, the nucleic acids encoding FoxP3 and the CAR are separated and separately transcribed as separate expression constructs. Separable proteins may also result from post-transcriptional modification or cleavage, for example, by the inclusion of a 2A self-cleaving peptide. In some embodiments, the nucleic acids encoding FoxP3 and the CAR may be linked by a nucleic acid sequence encoding a 2A self-cleaving peptide.

### Vectors

In some aspects, the disclosure relates to nucleic acids of comprising a vector. The term “vector” refers to a nucleic acid construct useful for transfer of genetic material onto a cell. A vector may comprise a nucleic acid construct in single-stranded or double-stranded form, and may comprise additional molecules, for example, DNA-associated proteins or viral capsid or envelope proteins. Vectors for eukaryotic and prokaryotic cells are well known to those in the art and include, for example, linear and circular DNA or RNA (e.g., plasmids), viral vectors (e.g., retroviral and parvoviral vectors, such as lentivirus-derived, Moloney murine leukemia virus-derived, adenovirus-derived, and AAV-derived vectors).

In some embodiments, the vector comprises a viral capsid. In some embodiments, the viral capsid is an adeno-associated viral (AAV) capsid protein. In some embodiments, the AAV is of serotype AAV8. In some embodiments, viral capsid protein is encoded by an nucleic acid sequence having at least 70% identity (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to the sequence of SEQ ID NO: 37. In some embodiments, the viral capsid protein is encoded by a nucleic acid sequence comprising a sequence of SEQ ID NO: 37. In some embodiments, the AAV is of serotype AAV8. In some embodiments, viral capsid protein is encoded by an nucleic acid sequence having at least 70% identity (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to the sequence of SEQ ID NO: 38. In some embodiments, the viral capsid protein is encoded by a nucleic acid sequence comprising a sequence of SEQ ID NO: 38.

In some embodiments, the vector is a plasmid. In some embodiments, the vector carrying any nucleic acids disclosed herein may be a plasmid. Plasmids are well known in the art, but are generally known to be small DNA molecules which are independent and separate from the chromosomal DNA, and are also self-replicating. Plasmids are most commonly found to be small circular double stranded DNA molecules, which typically do not carry any essential nucleic acid sequences (e.g., genes), but rather carry auxiliary or acquired genes such as nucleic acids encoding antibiotic resistance genes as well as transgenes incorporated artificially. Plasmids (both natural and artificial) are very useful and common delivery mechanisms (e.g., vectors) in molecular cloning for their ability to introduce foreign nucleic acids into a host cell. At their most basic, a plasmid vector will comprise an origin of replication, promoter region, and insert (e.g. transgene).

In some embodiments, the vector carrying any nucleic acid as disclosed herein is a viral vector. Viruses used as vectors are well known in the art, but generally known to be selected for their various characteristics and ability to introduce foreign material (e.g., nucleic acids, genes) into a separate organism, nucleic acid, or genome. Some viruses may carry and insert plasmids into the organism and some may have the ability to integrate foreign genetic material into the organism's DNA. In some embodiments, the viral vector is selected a retroviral vector (e.g., lentiviral vector), adenoviral (Ad) vector, or an Adeno-associated viral vector (e.g., AAV vector). In some embodiments, the viral vector is an AAV vector. In some embodiments, the viral vector is an adenoviral (Ad) vector. In some embodiments, the vector is a lentiviral vector.

In some embodiments, the expression construct is flanked by viral long terminal repeats (LTRs). In some embodiments, the LTRs are retroviral LTRs. In some embodiments, the LTRs are HIV LTRs. The nucleic acids disclosed herein may comprise a 5′ long terminal repeat (LTR) and a 3′ LTR. The 5′ LTR and/or 3′ LTR may be the native 5′ LTR and native 3′ LTR of a viral genome. Alternatively, either one may be modified, (e.g., including deletions, insertions, and/or mutations) relative to the native sequences. In some examples, the 3′ LTR may further comprise a polyadenylation (e.g., AAUAAA) (polyA) enhancer signal sequence, which is located upstream of the cleavage/polyA site and function to increase the polyA site efficiency and thus polyadenylation efficiency. Exemplary polyadenylation enhancer signal sequences include upstream sequence element (USE) from a suitable viral gene, for example, simian virus 40 (SV40) late gene or human immunodeficiency virus (HIV). Inclusion of such a polyA enhancer signal sequence may facilitate transcription termination and reduce read-through of vector transcript and improving packaging efficiency, which would lead to increased viral titer. In some embodiments, the LTRs may be native to the viral vector used. In some embodiments, the LTRs may be HIV LTRs.

In some embodiments, the vector is an adeno-associated viral (AAV) based vector. Adeno-associated virus (AAV) is a small (20 nm) replication-defective, nonenveloped DNA virus, that depends on the presence of a second virus, for example, adenovirus or herpesvirus, for productive infection. AAV is not known to cause disease and induces a very mild immune response. AAV can infect both dividing and non-dividing cells and stably incorporates its genome into that of the host cell. Various serotypes of AAV are known in the art. AAV serotype affects tissue tropism of the respective viral particles and allows to target specific cell types or tissues, making AAV vectors attractive for in vivo gene delivery applications in which only a specific cell type or tissue is targeted and/or gene transfer into non-targeted cells or tissues is not desirable. Wild type AAV particles harbor a single-stranded DNA genome comprising two genes: The AAV rep gene encodes proteins controlling viral replication, structural gene expression, and integration into the host genome. The AAV cap gene encodes capsid structural proteins. The 5′ and 3′ termini each comprise an inverted terminal repeat region (ITR), which is involved in multiplication of the AAV genome. In some embodiments, an AAV ITR sequence comprises 145 nucleotides. In general, an AAV ITR sequence is a self-complementary nucleic acid structure that is able to form a hairpin, which plays a role in AAV self-priming for synthesis of the second DNA AAV strand during the viral life cycle. In some embodiments, the disclosure relates to an AAV vector comprising the nucleic acids as disclosed herein flanked by AAV ITRs. In some embodiments, the capsid protein is an AAV1, AAV2, AAV3, AAV3b, AAV4, AAV5, AAV6, AAV7, AAV8, AAVrh.8, AAV9, AAV10, AAVrh.10, AAVrh.39, AAVrh.43, AAV.PHPB, AAV.PHPB.e, AAVrh32.33, or a variant thereof, capsid protein.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned. The present invention also provides vectors in which a DNA of the present invention is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. The vectors can be suitable for replication and integration into eukaryotes. Typical cloning vectors contain transcription and translation terminators, initiation sequences, and promoters useful for regulation of the expression of the desired nucleic acid sequence. The expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector. The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like, and fluorescent genes such as GFP, YFP, RFP and the like. In some embodiments, reporter genes or selectable marker genes are excluded from a CAR polypeptide used in a therapy as described herein. Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property (e.g., enzymatic activity, antibiotic resistance, fluorescence). Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter driven transcription. Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means. In some embodiments, the host cell is a T cell.

Physical methods for introducing a polynucleotide into a host cell include calcium phosphate precipitation, lipofection, particle bombardment, microinjection, electroporation, electroporation in addition to a plasmid, and the like. Other suitable methods include the use of transposons (natural and synthetic, for example Sleeping Beauty transposon method), ssDNA, and circular ssDNA. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection. Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362. Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid.

In some embodiments, the disclosure teaches a vector carrying a nucleic acid as described herein, where the vector comprises a nucleic acid sequence of SEQ ID NO: 1. In some embodiments, the disclosure teaches a vector carrying a nucleic acid encoding a CAR as described herein. In some embodiments, the CAR has an antigen binding domain comprising a nucleic acid, wherein the nucleic acid has at least 70% (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) and all increments thereof (e.g., tenths of a percent (i.e., 0.1%), hundredths of a percent (i.e., 0.01%), etc.) identity to the sequence of SEQ ID NO: 22 or 32-33. In some embodiments, the CAR has an antigen binding domain comprising a nucleic acid, wherein the nucleic acid has a sequence of SEQ ID NO: 22 or 32-33. In some embodiments, the CAR has transmembrane domain comprising a nucleic acid, wherein the nucleic acid has at least 70% (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) and all increments thereof (e.g., tenths of a percent (i.e., 0.1%), hundredths of a percent (i.e., 0.01%), etc.) identity to the sequence of SEQ ID NO: 25. In some embodiments, the CAR has an transmembrane domain comprising a nucleic acid, wherein the nucleic acid has a sequence of SEQ ID NO: 25. In some embodiments, the CAR has cytoplasmic signaling domain comprising a nucleic acid, wherein the nucleic acid has at least 70% (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) and all increments thereof (e.g., tenths of a percent (i.e., 0.1%), hundredths of a percent (i.e., 0.01%), etc.) identity to the sequence of SEQ ID NO: 26-29. In some embodiments, the CAR has an cytoplasmic signaling domain comprising a nucleic acid, wherein the nucleic acid has a sequence of SEQ ID NO: 26-29.

In some embodiments, the disclosure teaches a vector carrying a nucleic acid as described herein, where the vector comprises a nucleic acid sequence with at least 70% (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) and all increments thereof (e.g., tenths of a percent (i.e., 0.1%), hundredths of a percent (i.e., 0.01%), etc.) identity to of any of the sequences SEQ ID NOs: 1-4. In some embodiments, the disclosure teaches a vector carrying a nucleic acid as described herein, where the vector comprises a nucleic acid sequence of any of the sequences SEQ ID NOs: 1-4. In some embodiments, the disclosure teaches a vector carrying a nucleic acid as described herein, where the vector comprises a nucleic acid with at least 70% (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) and all increments thereof (e.g., tenths of a percent (i.e., 0.1%), hundredths of a percent (i.e., 0.01%), etc.) identity to the sequence of SEQ ID NO: 1. In some embodiments, the disclosure teaches a vector carrying a nucleic acid as described herein, where the vector comprises a nucleic acid with at least 70% (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) and all increments thereof (e.g., tenths of a percent (i.e., 0.1%), hundredths of a percent (i.e., 0.01%), etc.) identity to the sequence of SEQ ID NO: 2. In some embodiments, the disclosure teaches a vector carrying a nucleic acid as described herein, where the vector comprises a nucleic acid sequence of SEQ ID NO: 2. In some embodiments, the disclosure teaches a vector carrying a nucleic acid as described herein, where the vector comprises a nucleic acid with at least 70% (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) and all increments thereof (e.g., tenths of a percent (i.e., 0.1%), hundredths of a percent (i.e., 0.01%), etc.) identity to the sequence of SEQ ID NO: 3. In some embodiments, the disclosure teaches a vector carrying a nucleic acid as described herein, where the vector comprises a nucleic acid sequence of SEQ ID NO: 3. In some embodiments, the disclosure teaches a vector carrying a nucleic acid as described herein, where the vector comprises a nucleic acid with at least 70% (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) and all increments thereof (e.g., tenths of a percent (i.e., 0.1%), hundredths of a percent (i.e., 0.01%), etc.) identity to the sequence of SEQ ID NO: 4. In some embodiments, the disclosure teaches a vector carrying a nucleic acid as described herein, where the vector comprises a nucleic acid sequence of SEQ ID NO: 4.

### Cells

In an aspect, the disclosure relates to a host cell comprising the nucleic acids and/or vectors as disclosed herein. The term “host cell” generally refers to any cell from a living organism which holds, carries, or is infected with material of another organism (e.g., human cell transduced with a virus carrying a transgene). Suitable host cells may be readily selected by one of skill in the art in view of the indication for which the application is directed. For example, one suitable host cell is a mammalian immune cell. The cells may be autologous (i.e. obtained from the same subject into which they will be returned) or allogenic (i.e. derived from a subject other than the subject they are to be given, but of the same species). In some embodiments, host cell may be mammalian (e.g., a human, mouse, rat, cat, dog, sheep, rabbit, horse, cow, goat, pig, guinea pig, hamster, chicken, turkey, or a non-human primate (e.g., Marmoset, Macaque)). In some embodiments, the mammalian cell is a human cell. Host cells or tissues can be isolated from a subject by any suitable method. For example, cells or tissues may be isolated by surgery, biopsy (e.g., biopsy of skin tissue, lung tissue, liver tissue, adipose tissue), or collection of biological fluids such as blood. In some embodiments, cells are isolated from bone marrow. In some embodiments, cells are isolated from adipose tissue. In some embodiments, cells are isolated from a lipoaspirate. Appropriate methods for isolating cells from adipose tissue for ex vivo transfection are known in the art. See, e.g., Kuroda, M., et al., (2011), Journal of Diabetes Investigation, 2: 333-340; Kouki Morizono, et al. Human Gene Therapy. January 2003, 14(1): 59-66; and Patricia A. Zuk, Viral Transduction of Adipose-Derived Stem Cells, Methods in Molecular Biology, 1, Volume 702, Adipose-Derived Stem Cells, Part 4, Pages 345-357.

In some embodiments, the host cell is an immune cell. Immune cells (e.g., leukocytes) are well known in the art and generally known to constitute cells which comprise the immune system and mediate the immune response of a living organism. Examples of immune cells are lymphocytes (e.g., T-cells, T-regulatory cells (T-regs), and NK-cells), phagocytes (e.g., monocytes and macrophages), granulocytes (e.g., neutrophils, basophils and eosinophils), and dendritic cells. In some embodiments, the disclosure relates to a plurality of the host cells disclosed herein. The plurality of cells may be greater than 2 (e.g., 10; 100; 1,000; 10,000; 10⁶; 10⁹; 10¹², or more) host cells. The plurality of cells should be substantially pure, meaning the plurality of cells are substantially free of other cells (e.g., lineages, cells containing other foreign nucleic acids, or otherwise contaminated), proteins, and/or agents.

In some embodiments, the immune cells are T-cells. In some embodiments, the immune cells are T-regs. T-cells and T-regs are well known in the art, but are known as a subtypes of white blood cells (e.g., leukocytes) and play a central role in cell-mediated immunity. T-cells are a form of lymphocytes which develop in the thymus, contain T-cell receptor (TCR), and either modulate an immune response upon recognition of a ligand or directly kill a ligand presenting cell. T-regs (formerly known as suppressors cells) are a subset of T-cells (which are a subset of leukocytes), which generally modulate the immune reaction of a subject by suppressing and downregulating the induction and proliferation of T-cells. In doing so, they inhibit cytotoxic effects of T-cells due to T-reg recognition of an antigen.

### Compositions

In some aspects, the disclosure relates to a composition comprising the nucleic acids, vectors, host cells, or combination thereof, as disclosed herein. In some embodiments, the host cells or nucleic acids disclosed herein may be in a composition, including the pharmaceutical forms suitable for injectable use include sterile aqueous solutions or dispersions and sterile powders for the extemporaneous preparation of sterile injectable solutions or dispersions.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the nucleic acid or active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In some embodiments, the composition comprises a plurality of the nucleic acids, vectors, host cells, or a combination thereof, as disclosed herein. The nucleic acids or host cells of the instant disclosure may be delivered to a subject in compositions according to any appropriate methods known in the art. The compositions of the invention may comprise a nucleic acid or host cell alone, or in combination with one or more other nucleic acids or host cells (e.g., a second nucleic acid or host cell encoding having one or more different nucleic acid (e.g., CAR). In some embodiments, the composition may contain a carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. Suitable carriers may be readily selected by one of skill in the art in view of the indication for which the nucleic acid or host cell is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. Still others will be apparent to the skilled artisan. Optionally, the compositions of the invention may contain, in addition to the nucleic acid or host cell and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present invention into suitable host cells. In particular, the rAAV vector delivered transgenes may be formulated for delivery either encapsulated in a lipid particle, a liposome, a vesicle, a nanosphere, or a nanoparticle or the like. In some embodiments, the compositions of the disclosure are delivered by a delivery vehicle, including but not limited to, liposomes, nanocapsules, microparticles, microspheres, lipid particles, vesicles, and the like.

Such formulations may be preferred for the introduction of pharmaceutically acceptable formulations of the nucleic acids, vectors, or host cells disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

In some embodiments, the excipient is a cryoprotectant. A cryoprotectant is a substance used to protect tissues or samples (e.g., nucleic acids, vectors, host cells) from damage caused or related to cold temperatures (e.g., freezing, temperatures below ambient). For example damage can occur by a variety of mechanisms, but often is the result of either mechanical damage to the cell by the distortion caused by ice crystal formation, or chemical damage due to changes in the solutes surrounding the cells (e.g., dehydration due to increased salt concentration due extracellular fluids freezing). Cryoprotectants work by lowering the melting point of water, thereby increasing the ranges of temperatures cells remain viable and avoid the issues surrounding cold temperatures. Suitable cryoprotectants may be readily selected by one of skill in the art in view of the indication for which the nucleic acid or host cell is directed. For example, one suitable cryoprotectant includes glycerol, which may be formulated with a variety of buffering solutions (e.g., phosphate buffered saline). Other exemplary cryoprotectants include: sucrose, trehalose, dextrose, polyvinylpyrrolidone (PVP), methylcellulose, dimethyl sulfoxide (DMSO), ethylene glycol, polyethylene glycol (PEG), and propylene glycol.

### Methods of Manufacture

In some aspects, the disclosure relates to methods of making the CAR T-cells and CAR T-reg cells as disclosed herein.

In some embodiments T-cells are transfected with the CARs as disclosed herein and following transfection are co-cultured with Hela cells. In some embodiments T-cells are transfected with the CARs as disclosed herein and following transfection are co-cultured with peripheral blood mononuclear cells (PBMCs). In some embodiments T-cells are transfected with the CARs as disclosed herein and following transfection are co-cultured with Hela cells and PBMCs. In some embodiments T-cells are transfected with the CARs as disclosed herein and following transfection are co-cultured with IL-7, IL21, IL15 cytokines, and anti-CD3 antibodies. In some embodiments T-cells are transfected with the CARs as disclosed herein and following transfection are co-cultured with Hela cells and IL-7, IL21, IL15 cytokines, and anti-CD3 antibodies. In some embodiments T-cells are transfected with the CARs as disclosed herein and following transfection are co-cultured with peripheral blood mononuclear cells (PBMCs) and IL-7, IL21, IL15 cytokines, and anti-CD3 antibodies. In some embodiments T-cells are transfected with the CARs as disclosed herein and following transfection are co-cultured with Hela cells, peripheral blood mononuclear cells (PBMCs), and IL-7, IL21, IL15 cytokines and anti-CD3 antibodies.

In some embodiments, the T-cells are transfected at least 12 hours prior to co-culturing. In some embodiments, the T-cells are transfected at least 18 hours prior to co-culturing. In some embodiments, the T-cells are transfected at least 24 hours prior to co-culturing. In some embodiments, the T-cells are co-cultured with at least 1×10⁵ Hela cells. In some embodiments, the T-cells are co-cultured with at least 1×10⁶ Hela cells. In some embodiments, the T-cells are co-cultured with at least 1×10⁷ Hela cells. In some embodiments, the T-cells are co-cultured with at least 1×10⁸ Hela cells. In some embodiments, the Hela cells are irradiated prior to co-culturing. In some embodiments, the Hela cells are irradiated with about 100 gray (gy) of radiation prior to co-culturing. In some embodiments, the Hela cells are irradiated with about 125 gy of radiation prior to co-culturing. In some embodiments, the Hela cells are irradiated with about 135 gy of radiation prior to co-culturing. In some embodiments, the Hela cells express an AAV capsid protein. In some embodiments, the Hela cells express an AAV1 capsid protein. In some embodiments, the Hela cells express an AAV2 capsid protein. In some embodiments, the Hela cells express an AAV3b capsid protein. In some embodiments, the Hela cells express an AAV6 capsid protein. In some embodiments, the Hela cells express an AAV7 capsid protein. In some embodiments, the Hela cells express an AAV8 capsid protein. In some embodiments, the Hela cells express an AAV9 capsid protein. In some embodiments, the Hela cells express an AAV10 capsid protein. In some embodiments, the Hela cells express an AAV-rh32.331 capsid protein.

In some embodiments, the T-cells are co-cultured with at least 5×10⁵ peripheral blood mononuclear cell (PBMCs). In some embodiments, the T-cells are co-cultured with at least 5×10⁶ PBMCs. In some embodiments, the T-cells are co-cultured with at least 5×10⁷ PBMCs. In some embodiments, the T-cells are co-cultured with at least 5×10⁸ PBMCs. In some embodiments, the PBMCs are irradiated prior to co-culturing. In some embodiments, the PBMCs are irradiated with about 20 gy of radiation prior to co-culturing. In some embodiments, the PBMCs are irradiated with about 30 gy of radiation prior to co-culturing. In some embodiments, the PBMCs are irradiated with about 35 gy of radiation prior to co-culturing.

In some embodiments, the IL-7 cytokines are co-cultured at a concentration of about 2 nanograms (ng) per milliliter (ml) (ng/ml). In some embodiments, the IL-7 cytokines are co-cultured at a concentration of about 3 ng/ml. In some embodiments, the IL-7 cytokines are co-cultured at a concentration of about 4 ng/ml. In some embodiments, the IL-7 cytokines are co-cultured at a concentration of about 5 ng/ml. In some embodiments, the IL-7 cytokines are co-cultured at a concentration of about 6 ng/ml. In some embodiments, the IL-7 cytokines are co-cultured at a concentration of about 7 ng/ml.

In some embodiments, the IL21 cytokines are co-cultured at a concentration of about 15 ng/ml. In some embodiments, the IL21 cytokines are co-cultured at a concentration of about 20 ng/ml. In some embodiments, the IL21 cytokines are co-cultured at a concentration of about 23 ng/ml. In some embodiments, the IL21 cytokines are co-cultured at a concentration of about 25 ng/ml. In some embodiments, the IL21 cytokines are co-cultured at a concentration of about 27 ng/ml. In some embodiments, the IL21 cytokines are co-cultured at a concentration of about 30 ng/ml.

In some embodiments, the IL15 cytokines are co-cultured at a concentration of about 0.2 ng/ml. In some embodiments, the IL15 cytokines are co-cultured at a concentration of about 0.3 ng/ml. In some embodiments, the IL15 cytokines are co-cultured at a concentration of about 0.4 ng/ml. In some embodiments, the IL15 cytokines are co-cultured at a concentration of about 0.5 ng/ml. In some embodiments, the IL15 cytokines are co-cultured at a concentration of about 0.6 ng/ml. In some embodiments, the IL15 cytokines are co-cultured at a concentration of about 0.7 ng/ml.

In some embodiments, the anti-CD3 antibodies are co-cultured at a concentration of about 20 ng/ml. In some embodiments, the anti-CD3 antibodies are co-cultured at a concentration of about 25 ng/ml. In some embodiments, the anti-CD3 antibodies are co-cultured at a concentration of about 27 ng/ml. In some embodiments, the anti-CD3 antibodies are co-cultured at a concentration of about 30 ng/ml. In some embodiments, the anti-CD3 antibodies are co-cultured at a concentration of about 33 ng/ml. In some embodiments, the anti-CD3 antibodies are co-cultured at a concentration of about 35 ng/ml.

In some embodiments, the media is changed at least every 2 days. In some embodiments, a portion of the media is changed. In some embodiments, about two-thirds of the media is changed about every 2 days. In some embodiments, the new media contains new cytokines and/or antibodies.

In some embodiments T-reg cells are transfected with the CARs as disclosed herein and following transfection are co-cultured with Hela cells. In some embodiments T-reg cells are transfected with the CARs as disclosed herein and following transfection are co-cultured with peripheral blood mononuclear cells (PBMCs). In some embodiments T-reg cells are transfected with the CARs as disclosed herein and following transfection are co-cultured with Hela cells and PBMCs. In some embodiments T-reg cells are transfected with the CARs as disclosed herein and following transfection are co-cultured with IL-7, IL21, IL15 cytokines, TGF-beta, and anti-CD3 antibodies. In some embodiments T-reg cells are transfected with the CARs as disclosed herein and following transfection are co-cultured with Hela cells and IL-7, IL21, IL15 cytokines, TGF-beta, and anti-CD3 antibodies. In some embodiments T-reg cells are transfected with the CARs as disclosed herein and following transfection are co-cultured with peripheral blood mononuclear cells (PBMCs) and IL-7, IL21, IL15 cytokines, TGF-beta, and anti-CD3 antibodies. In some embodiments T-reg cells are transfected with the CARs as disclosed herein and following transfection are co-cultured with Hela cells, peripheral blood mononuclear cells (PBMCs), and IL-7, IL21, IL15 cytokines, TGF-beta, and anti-CD3 antibodies.

In some embodiments, the T-reg cells are transfected at least 12 hours prior to co-culturing. In some embodiments, the T-reg cells are transfected at least 18 hours prior to co-culturing. In some embodiments, the T-reg cells are transfected at least 24 hours prior to co-culturing. In some embodiments, the T-reg cells are co-cultured with at least 1×10⁵ Hela cells. In some embodiments, the T-reg cells are co-cultured with at least 1×10⁶ Hela cells. In some embodiments, the T-reg cells are co-cultured with at least 1×10⁷ Hela cells. In some embodiments, the T-reg cells are co-cultured with at least 1×10⁸ Hela cells. In some embodiments, the Hela cells are irradiated prior to co-culturing. In some embodiments, the Hela cells are irradiated with about 100 gray (gy) of radiation prior to co-culturing. In some embodiments, the Hela cells are irradiated with about 125 gy of radiation prior to co-culturing. In some embodiments, the Hela cells are irradiated with about 135 gy of radiation prior to co-culturing. In some embodiments, the Hela cells express an AAV capsid protein. In some embodiments, the Hela cells express an AAV1 capsid protein. In some embodiments, the Hela cells express an AAV2 capsid protein. In some embodiments, the Hela cells express an AAV3b capsid protein. In some embodiments, the Hela cells express an AAV6 capsid protein. In some embodiments, the Hela cells express an AAV7 capsid protein. In some embodiments, the Hela cells express an AAV8 capsid protein. In some embodiments, the Hela cells express an AAV9 capsid protein. In some embodiments, the Hela cells express an AAV10 capsid protein. In some embodiments, the Hela cells express an AAV-rh32.331 capsid protein.

In some embodiments, the T-reg cells are co-cultured with at least 5×10⁵ PBMCs. In some embodiments, the T-reg cells are co-cultured with at least 5×10⁶ PBMCs. In some embodiments, the T-reg cells are co-cultured with at least 5×10⁷ PBMCs. In some embodiments, the T-reg cells are co-cultured with at least 5×10⁸ PBMCs. In some embodiments, the PBMCs are irradiated prior to co-culturing. In some embodiments, the PBMCs are irradiated with about 20 gy of radiation prior to co-culturing. In some embodiments, the PBMCs are irradiated with about 30 gy of radiation prior to co-culturing. In some embodiments, the PBMCs are irradiated with about 35 gy of radiation prior to co-culturing.

In some embodiments, the IL-7 cytokines are co-cultured at a concentration of about 2 nanograms (ng) per milliliter (ml) (ng/ml). In some embodiments, the IL-7 cytokines are co-cultured at a concentration of about 3 ng/ml. In some embodiments, the IL-7 cytokines are co-cultured at a concentration of about 4 ng/ml. In some embodiments, the IL-7 cytokines are co-cultured at a concentration of about 5 ng/ml. In some embodiments, the IL-7 cytokines are co-cultured at a concentration of about 6 ng/ml. In some embodiments, the IL-7 cytokines are co-cultured at a concentration of about 7 ng/ml.

In some embodiments, the IL21 cytokines are co-cultured at a concentration of about 15 ng/ml. In some embodiments, the IL21 cytokines are co-cultured at a concentration of about 20 ng/ml. In some embodiments, the IL21 cytokines are co-cultured at a concentration of about 23 ng/ml. In some embodiments, the IL21 cytokines are co-cultured at a concentration of about 25 ng/ml. In some embodiments, the IL21 cytokines are co-cultured at a concentration of about 27 ng/ml. In some embodiments, the IL21 cytokines are co-cultured at a concentration of about 30 ng/ml.

In some embodiments, the IL15 cytokines are co-cultured at a concentration of about 0.2 ng/ml. In some embodiments, the IL15 cytokines are co-cultured at a concentration of about 0.3 ng/ml. In some embodiments, the IL15 cytokines are co-cultured at a concentration of about 0.4 ng/ml. In some embodiments, the IL15 cytokines are co-cultured at a concentration of about 0.5 ng/ml. In some embodiments, the IL15 cytokines are co-cultured at a concentration of about 0.6 ng/ml. In some embodiments, the IL15 cytokines are co-cultured at a concentration of about 0.7 ng/ml.

In some embodiments, the TGF-beta is co-cultured at a concentration of about 2 ng/ml. In some embodiments, the TGF-beta is co-cultured at a concentration of about 3 ng/ml. In some embodiments, the TGF-beta is co-cultured at a concentration of about 4 ng/ml. In some embodiments, the TGF-beta is co-cultured at a concentration of about 5 ng/ml. In some embodiments, the TGF-beta is co-cultured at a concentration of about 6 ng/ml. In some embodiments, the TGF-beta is co-cultured at a concentration of about 7 ng/ml.

In some embodiments, the anti-CD3 antibodies are co-cultured at a concentration of about 20 ng/ml. In some embodiments, the anti-CD3 antibodies are co-cultured at a concentration of about 25 ng/ml. In some embodiments, the anti-CD3 antibodies are co-cultured at a concentration of about 27 ng/ml. In some embodiments, the anti-CD3 antibodies are co-cultured at a concentration of about 30 ng/ml. In some embodiments, the anti-CD3 antibodies are co-cultured at a concentration of about 33 ng/ml. In some embodiments, the anti-CD3 antibodies are co-cultured at a concentration of about 35 ng/ml.

In some embodiments, the media is changed at least every 2 days. In some embodiments, a portion of the media is changed. In some embodiments, about two-thirds of the media is changed about every 2 days. In some embodiments, the new media contains new cytokines and/or antibodies.

### Administration

In some embodiments, the nucleic acids carried by a vector or host cells, which may be suspended in a physiologically compatible carrier (e.g., in a composition), may be administered to a subject (e.g., a human, mouse, rat, cat, dog, sheep, rabbit, horse, cow, goat, pig, guinea pig, hamster, chicken, turkey, or a non-human primate (e.g., Marmoset, Macaque)).

Administration of the nucleic acids, vectors, and/or host cells as described herein may be used to modulate the immune reaction (i.e., response) to target antigens (e.g., AAV capsid proteins). For example, by designing a CAR in a T-cell to recognize target antigens (e.g., AAV capsid proteins) the T-cell can be used to kill the APCs presenting the antigen, thereby inhibiting the immune response related to the target antigen. In some embodiments, a CAR targeting an antigen is expressed in a CD8+ T-cell for use in suppressing the immune response related to the target antigen. Conversely, transcriptional regulators (e.g., FOXP3) can be used to create regulatory T cells (i.e., T-regs) for the opposite effect. In some embodiments, the a T-regulatory cell expresses a CAR designed to recognize a target antigen (e.g., AAV capsid). By introducing the CAR into a T-reg cell and expressing the transcriptional regulator FOXP3, the T-reg cell is designed and functions inhibit the killing of an APC presenting the target antigen, thereby suppressing any suppression of the immune system with respect to the target antigen (e.g., AAV capsid), in other words, promote or increase the immune response with respect to the target antigen (e.g., AAV capsid). In some embodiments, a CAR targeting an antigen is expressed in a CD4+ T-cell for use in promoting or increasing the immune response related to the target antigen. In some embodiments, a CAR targeting an antigen is expressed in a T-cell expressing FOXP3 for use in promoting or increasing the immune response related to the target antigen. In some embodiments, the CAR T-reg cells of the disclosure are used to modulate the immune response. In some embodiments, the modulation is of response to AAV capsids. In some embodiments, the modulation is performed in combination with steroid treatment. In some embodiments, the modulation is done in lieu of steroid treatment(s). In some embodiments, the modulation is performed to modulate previous administration of an AAV capsid. In some embodiments, the modulation is performed in a subject with a pre-existing immunity to an AAV or antigen. In some embodiments, the subject has circulating neutralizing antibodies against an AAV capsid protein. In some embodiments, the subject has previously been administered an AAV. In some embodiments, the subject has previously been administered a recombinant AAV (rAAV). In some embodiments, the subject has circulating neutralizing antibodies against an AAV capsid protein and has previously been administered an AAV and/or recombinant AAV (rAAV).

In some embodiments, the administration of the nucleic acids, vectors, and/or host cells as described herein may be used to modulate the expression or immune reaction to transgenes, for example in CRIM-negative subjects (see, Am J Med Genet C Semin Med Genet. 2012 Feb. 15; 160C(1):40-9. doi: 10.1002/ajmg.c.31319. Epub 2012 Jan. 17). In some embodiments, the transgenes are introduced by an rAAV. In some embodiments, the CAR T-cells are administered to reduce immunogenicity due to expression of the transgene. In some embodiments, the transgene is A1AT. In some embodiments, the nucleic acids, vectors, and/or host cells as described herein may be used to allow for the redosing of a subject with an AAV based therapy. For example, in some embodiments, the CAR T-cells are administered to increase the efficacy of a subsequent exposures or administrations of AAV based therapies. In some embodiments, the CAR T-reg cells are administered to decrease the efficacy of a subsequent exposures or administrations of AAV based therapies.

In some embodiments, the nucleic acids, vectors, and/or host cells as described herein may be used to modulate the immune reaction to gene editing tools. For example, in some embodiments, the CAR T-cells of the disclosure may be administered to decrease the immune response to gene editing tools (e.g., Cas proteins), introduced into a subject by an rAAV. In some embodiments, the CAR T-reg cells of the disclosure may be administered to increase the immune response to gene editing tools (e.g., Cas proteins), introduced into a subject by an rAAV. In some embodiments, the gene editing tool may be an AAV6+ CRISPR-Cas9 system, which may, in some embodiments, employ TRAC locus or genomic safe harbor based techniques.

In some embodiments, the nucleic acids, vectors, and/or host cells as described herein may be used in combination of a gene editing tool (e.g., CRISPR-Cas) to increase the expression of the CAR T-cells and CAR T-reg cells of the disclosure. In some embodiments, the increased expression allows for multiple re-administrations to a subject of AAV based therapies.

In some embodiments, administration of the nucleic acids, vectors, and/or host cells as described herein may be used to model immune reaction (i.e., response) to target antigens (e.g., AAV capsid proteins). As disclosed above, the CAR T-cells and CAR T-reg cells can be used to modulate the immune response, and thus can be designed to model a response to a given antigen in vitro.

In some embodiments, the CAR T-cells and CAR T-reg cells of the disclosure are used as adjuvant therapies or with an adjuvant therapy. The term “adjuvant,” as may be used herein, refers to any therapy or treatment (e.g., composition, drug, or method based) which is used as an adjunct to the primary or initial therapy or treatment. Adjuvants may be administered concurrently (e.g., at the same time, simultaneously) with the primary or initial treatment or shortly after the administration of the primary or initial treatment. In some, but not all, cases an adjuvant modulates (e.g., increases, decreases) the effect of the primary or initial treatment. In some, but not all, cases an adjuvant is used to modulate (e.g., increase, decrease) a side effect of the primary or initial treatment. In some, but not all, cases an adjuvant is used to prepare (e.g., condition) a subject in anticipation of the primary or initial treatment or aid in the primary or initial treatment's effects or sustain or aid in the recovery of the subject after the primary or initial treatment. In some embodiments, the CAR T-cells and CAR T-reg cells of the disclosure are administered to modulate the immune response to a given treatment. In some embodiments, the CAR T-cells and CAR T-reg cells of the disclosure are administered in connection with treatment of SMA. In some embodiments, the CAR T-cells and CAR T-reg cells of the disclosure are administered in connection with administration of Zolgensma. In some embodiments, the CAR T-cells and CAR T-reg cells of the disclosure are administered in connection with the treatment of Leber's congenital amaurosis. In some embodiments, the CAR T-cells and CAR T-reg cells of the disclosure are administered in connection with the administration of Luxturna.

In some embodiments, the CAR T-cells and CAR T-reg cells of the disclosure can be used to treat a disease or disorder. In some embodiments, the disease or disorder is an autoimmune or neuroinflammatory disease. In some embodiments, the disease or disorder is diabetes, multiple sclerosis, Amyotrophic lateral sclerosis, or Alzheimer's Disease.

Conventional and pharmaceutically acceptable routes of administration include, but are not limited to, direct delivery to the selected tissue (e.g., liver tissue, lung tissue) and administration subcutaneously, intraopancreatically, intranasally, parenterally, intravenously, intramuscularly, intrathecally, intracerebrally, orally, intraperitoneally, by inhalation or by another route. Routes of administration may be combined, if desired.

Delivery according the methods of the disclosure to a subject may be, for example, by administration into the bloodstream of the subject. Administration into the bloodstream may be by injection into a vein, an artery, or any other vascular conduit. Moreover, in certain instances, it may be desirable to deliver the nucleic acids, vectors, host cells, or a combination thereof to brain tissue, meninges, neuronal cells, glial cells, astrocytes, oligodendrocytes, cereobrospinal fluid (CSF), interstitial spaces and the like. In some embodiments, nucleic acids, vectors, host cells, or a combination thereof may be delivered directly to the spinal cord or brain (e.g., prefrontal cortex) by injection into the ventricular region, as well as to the striatum (e.g., the caudate nucleus or putamen of the striatum), and neuromuscular junction, or cerebellar lobule, with a needle, catheter or related device, using neurosurgical techniques known in the art, such as by stereotactic injection (see, e.g., Stein et al., J. Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000).

In certain circumstances it will be desirable to deliver the nucleic acids, vectors, host cells, or a combination thereof in suitably formulated pharmaceutical compositions disclosed herein either intrathecally, intracerebrally, intravenously, subcutaneously, intraopancreatically, intranasally, parenterally, intravenously, intramuscularly, orally, intraperitoneally, or by inhalation.

It can be appreciated by one skilled in the art that desirable administration of nucleic acids, vectors, host cells, or combination thereof can also include ex vivo administration. In some embodiments, ex vivo administration comprises (1) isolation of cells or tissue(s) of interest from a subject, (2) contacting the cells or tissue(s) with viral vectors in sufficient amounts to transfect the cells or tissue to provide sufficient levels of gene transfer and expression without undue adverse effect, and (3) transferring cells or tissue back into the subject. In some embodiments, cells or tissues may be cultured ex vivo for several days before and/or after transfection.

In an embodiment, cells or tissue(s) are transduced at a multiplicity of infection (MOI) of at least 10 infectious units (i.u.) of viral vectors per cell (for example: 10; 100; 1,000; 5,000; 10,000; 100,000 or more i.u.) or at a functionally equivalent viral copy number. In one embodiment, cells or tissue(s) are transduced at a MOI of 10 to 10,000 i.u. Routes for transfer of transfected cells or tissue(s) into a subject include, but are not limited to, subcutaneously, intraopancreatically, intranasally, parenterally, intravenously, intravascularly, intramuscularly, intrathecally, intracerebrally, intraperitoneally, or by inhalation. In some embodiments, transfected cells are administered by hepatic portal vein injection. In some embodiments, transfected cells are administered intravascularly. Methods for ex vivo administration of nucleic acids and/or vectors are well known in the art (see, e.g., Naldini, L. Nature Reviews Genetics (2011) 12, 301-315, Li, H. et al. Molecular Therapy (2010) 18, 1553-1558, and Loiler et al. Gene Therapy (2003) 10, 1551-1558).

The nucleic acids, vectors, host cells, or combination thereof of the disclosure may be administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected tissue (e.g., liver tissue, lung tissue) and administration subcutaneously, intraopancreatically, intranasally, parenterally, intravenously, intramuscularly, intrathecally, intracerebrally, orally, intraperitoneally, by inhalation or by another route. Routes of administration may be combined, if desired.

In some embodiments, the subject is a human. The human subject may be a male or female of any age (e.g., infant, child, adolescent, adult). In some embodiments, the subject has previously been administered a vector or host cell as described herein, or is contemplated to be administered a vector or host cell as described herein. In some embodiments, the subject has previously received administration of a vector or host cell as described herein, such as those disclosed herein. In some embodiments, the subject is contemplated to receive administration of a vector or host cell as described herein, such as those disclosed herein.

In an aspect, the disclosure relates to a method comprising administering to the subject a recombinant T-cell comprising a CAR, such that the immune response of the subject to the AAV capsid protein is inhibited. In some embodiments, the nucleic acids disclosed herein comprise a T-cell and CAR, which are administered to a subject such that the immune response to an AAV capsid protein is inhibited. By administering a T-cell comprising a CAR which recognizing an epitope of an AAV capsid protein, immune response may be modulated by recognizing antigen binding sites on circulating immune cells (e.g. APCs) which recognize the AAV epitope. It is believed these immune cells are reduced in number due to the cytotoxic effect of the CAR T-cells, thus lessening the immune response to any contemporaneously or subsequently administered cell comprising an AAV capsid protein epitope. The duration of the reduced immunity state with respect to the targeted epitope may be temporary or long lasting, the time to administration of any subsequent therapy will be timed to exploit the reduced immunity state, which will be readily apparent to one of ordinary skill without undue experimentation.

In an aspect the disclosure relates to a method comprising, administering to the subject a recombinant T-regulatory cell comprising a CAR, such that the immune response of the subject to the AAV capsid protein is induced. In some embodiments, the nucleic acids disclosed herein comprise a T-reg and CAR, which are administered to a subject such that the immune response to an AAV capsid protein is inhibited. By administering a T-reg comprising a CAR which recognizing an epitope of an AAV capsid protein, immune response may be modulated by recognizing antigen binding sites on circulating immune cells (e.g. APCs) which recognize the AAV epitope. It is believed these immune cells will reducing in number CAR T-cells targeting any APCs expressing the AAV epitope, thus increasing or restoring to baseline levels such APCs, thus increasing the immune response to any contemporaneously or subsequently administered cell comprising an AAV capsid protein epitope. The duration of the reduced immunity state with respect to the targeted epitope may be temporary or long lasting, the time to administration of any subsequent therapy will be timed to exploit the reduced immunity state, which will be readily apparent to one of ordinary skill without undue experimentation.

In an aspect, the disclosure relates to a method, comprising administering a therapeutic host cells to the subject. In some embodiments, the nucleic acids disclosed herein are administered to a subject therapeutically, or for the purpose of inducing a therapeutic effect. The dose of nucleic acids or host cells required to achieve a desired effect or “therapeutic effect,” e.g., the units of dose in vector genomes/per kilogram of body weight (vg/kg), will vary based on several factors including, but not limited to: the route of administration, the level of gene or RNA expression required to achieve a therapeutic effect, the specific disease or disorder being treated, and the stability of the gene or RNA product. One of skill in the art can readily determine an vector dose range to treat a subject having a particular disease or disorder based on the aforementioned factors, as well as other factors that are well known in the art. An effective amount of the nucleic acids is generally in the range of from about 10 μl to about 100 ml of solution containing from about 10⁹ to 10¹⁶ genome copies per subject. Other volumes of solution may be used. The volume used will typically depend, among other things, on the size of the subject, the dose of the nucleic acids, and the route of administration. For example, for intravenous administration a volume in range of 10 μl to 100 μl, 100 μl to 1 ml, 1 ml to 10 ml, or more may be used. In some cases, a dosage between about 10¹⁰ to 10¹² rAAV genome copies per subject is appropriate. In some embodiments the rAAV is administered at a dose of 10¹⁰, 10¹¹, 10¹², 10¹³, 10¹⁴, or 10¹⁵ genome copies per subject. In some embodiments the rAAV is administered at a dose of 10¹⁰, 10¹¹, 10¹², 10¹³, or 10¹⁴, genome copies per kg.

In some embodiments, the therapeutic rAAV comprises a capsid protein having the same serotype as the capsid protein targeted by the CAR. In some embodiments the rAAV has the same capsid protein as the capsid protein targeted by the nucleic acid expressing the CAR of the rAAV.

### Kits

The recombinant nucleic acids, compositions, vectors, host cells etc. described herein may, in some embodiments, be assembled into pharmaceutical or diagnostic or research kits to facilitate their use in therapeutic, diagnostic or research applications. A kit may include one or more containers housing the components of the invention and instructions for use. Specifically, such kits may include one or more agents described herein, along with instructions describing the intended application and the proper use of these agents. In certain embodiments agents in a kit may be in a pharmaceutical formulation and dosage suitable for a particular application and for a method of administration of the agents. Kits for research purposes may contain the components in appropriate concentrations or quantities for running various experiments.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the invention. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container. The kit may have one or more or all of the components required to administer the agents to a subject, such as a syringe, topical application devices, or intravenous needle tubing and bag.

Exemplary embodiments of the invention are described in more detail by the following examples. These embodiments are exemplary of the invention, which one skilled in the art will recognize is not limited to the exemplary embodiments.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1

This example describes CAR T-cell constructs that recognizes AAV capsid proteins. Two versions of CAR T-cell constructs were produced; one construct that is characterized by cytotoxic T-cell function, and another construct that is characterized by FoxP3 transcription regulator encoded as part of the CAR construct to create CAR T-regs against AAV capsid.

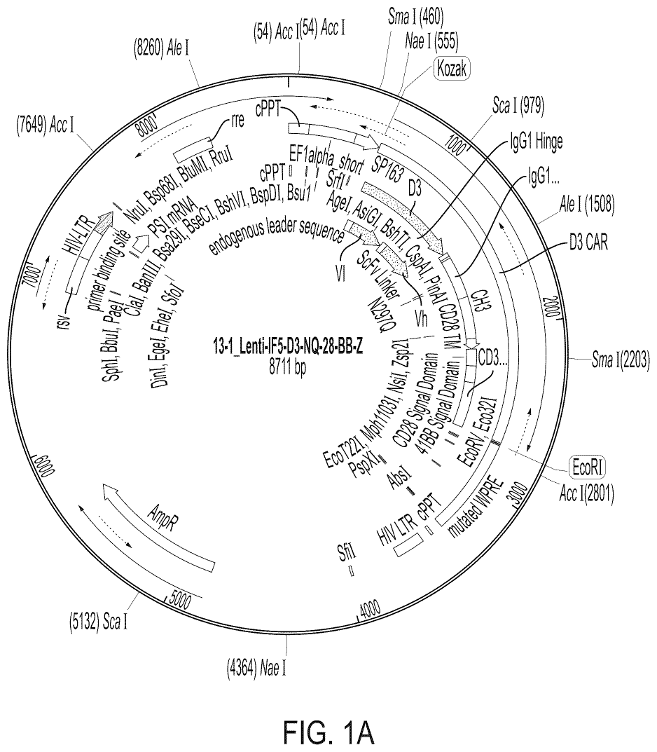

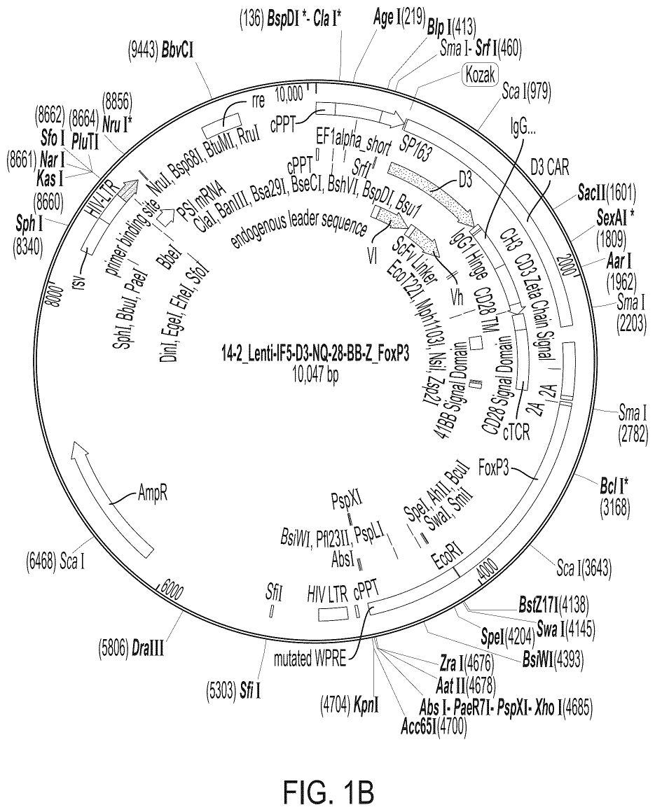

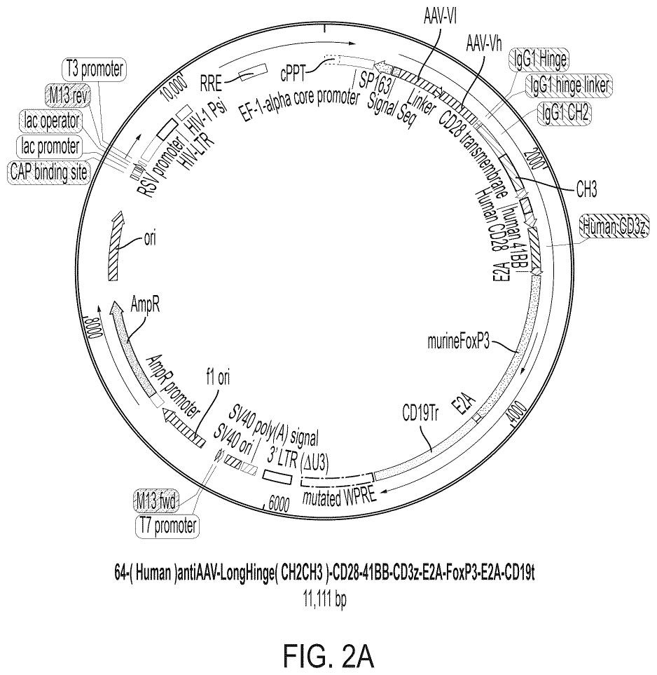

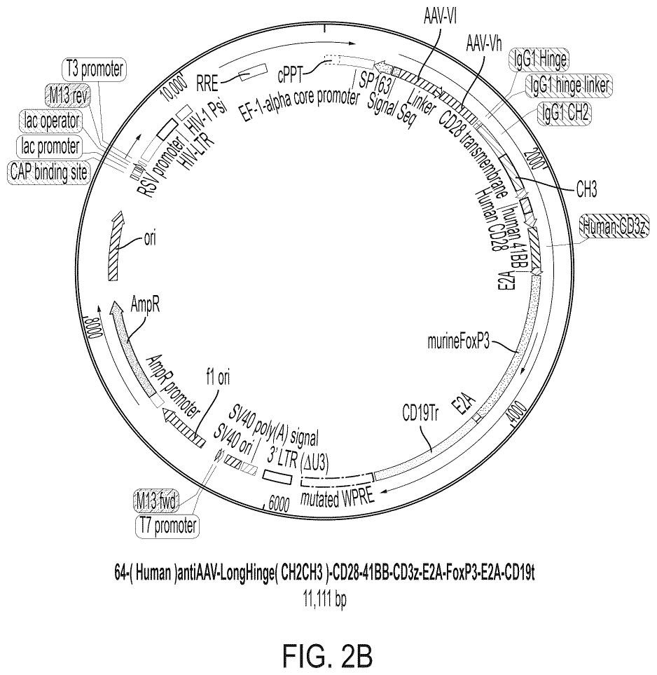

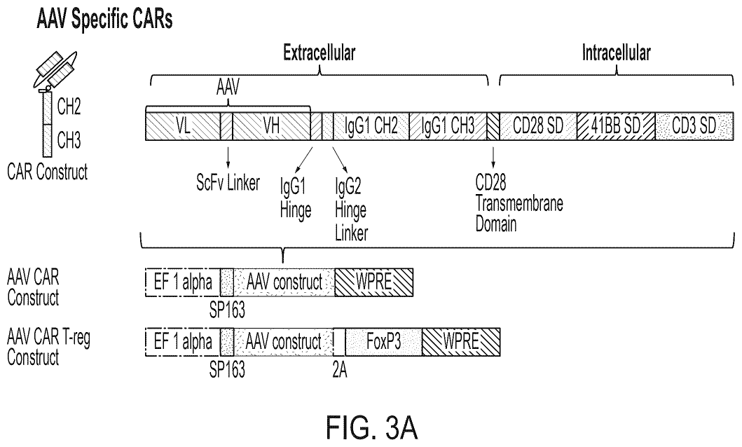

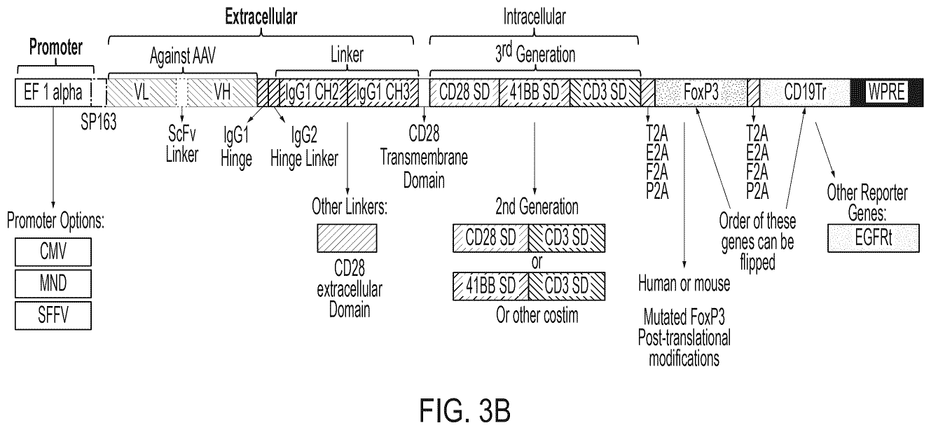

The CAR T-cell constructs comprise an anti-AAV2 capsid protein antibody (e.g., D3 antibody) and lack the iCaspase9 gene and 2A sequence, and a CD19 truncated sequence. Constructs were introduced into a lentiviral backbone and expressed from the EF1alpha promoter. In some embodiments, the constructs contain, endogenous leader sequence, D3 AAV specific antibody variable light and variable heavy domains with an scFv linker, a human IgG1 hinge and IgG2 linker, IgG1 CH2 and CH3 domains with the CD28 transmembrane domain, and internal CD28, 41BB and CD3 cytoplasmic signaling domains. The CAR T-regs construct further comprises a 2A self-cleaving sequence followed by the FoxP3 sequence. In some embodiments, the D3 endogenous leader sequence is replaced by a CD8 endogenous leader sequence. In some embodiments, a 2A self-cleaving sequence and EGFRt receptor sequence are added to the constructs. Examples of constructs described herein are shown in FIGS. 1-3.

Constructs were packaged into 3rd generation lentiviruses. Lentiviruses were transduced into human T-cells, isolated from human donor peripheral blood mononuclear cells (PBMCs), Jurkat cell lines (human T-cell line), and mouse T-cells. CAR T-regs against AAV were produced in two different ways. In some embodiments, CAR T-regs are isolated from human donors and then infected them with AAV CAR constructs. In some embodiments, CAR T-regs are produced by transducing cells with lentivirus encoding CAR that contains 2A and FoxP3, transcription regulator.

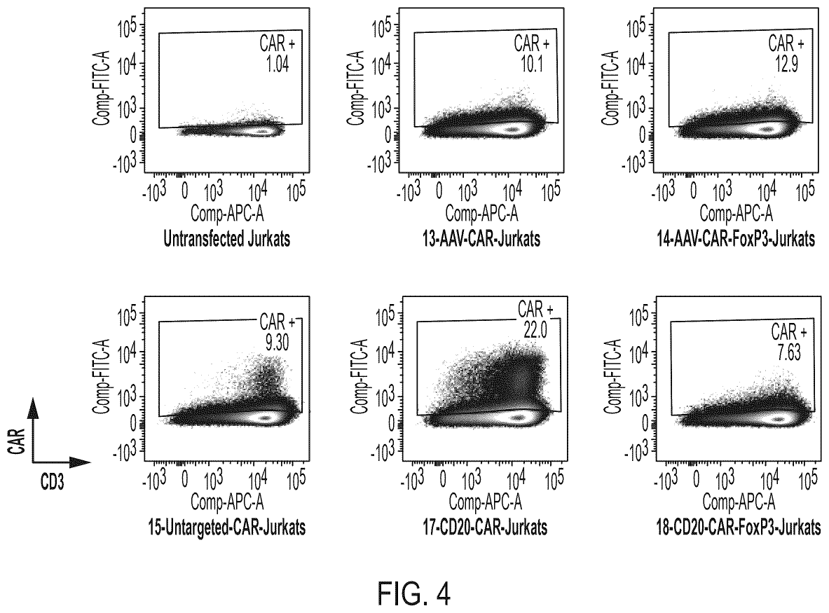

To determine the transduction efficiency of the lentiviral transduction of T-cells, CAR expression on the surface of the cell is analyzed using an antibody to detect the CH2-CH3 constant domain of the CAR, as shown in FIG. 4.

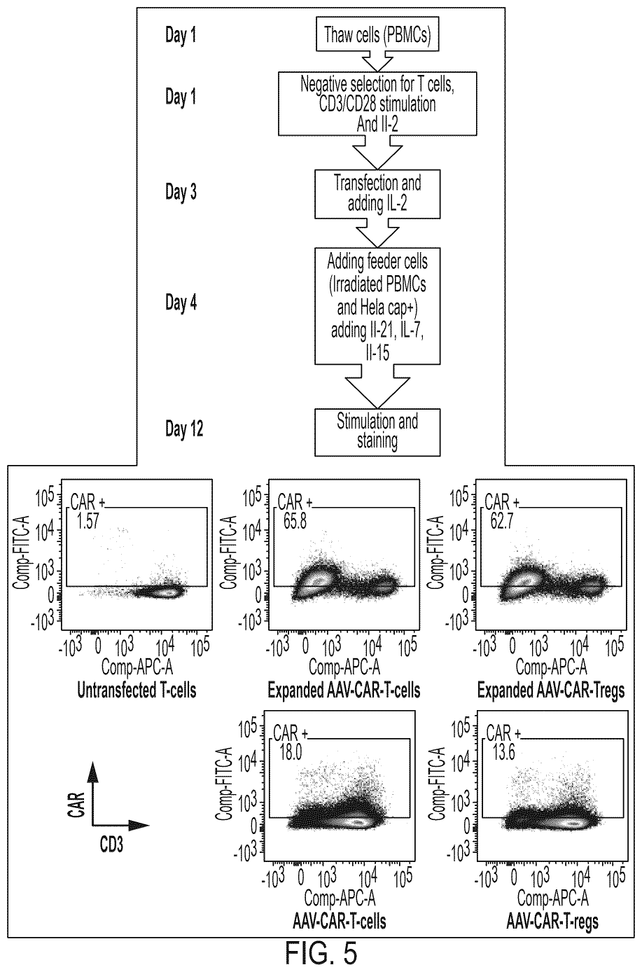

Cell transduction is enhanced by an expansion protocol. Briefly, after stimulation and transduction with lentivirus expressing CAR constructs, the cells are cultured with irradiated feeder cells (e.g., different cell lines than are being tested) in the presence of IL-2, 11-7 and IL-15 (FIG. 5). After 8 days of culturing under these conditions, expansion of CAR T-cells population has been observed to be 65% (FIG. 5). The purity of this population can further be enhanced by sorting based on the presence of EGFRt receptor.

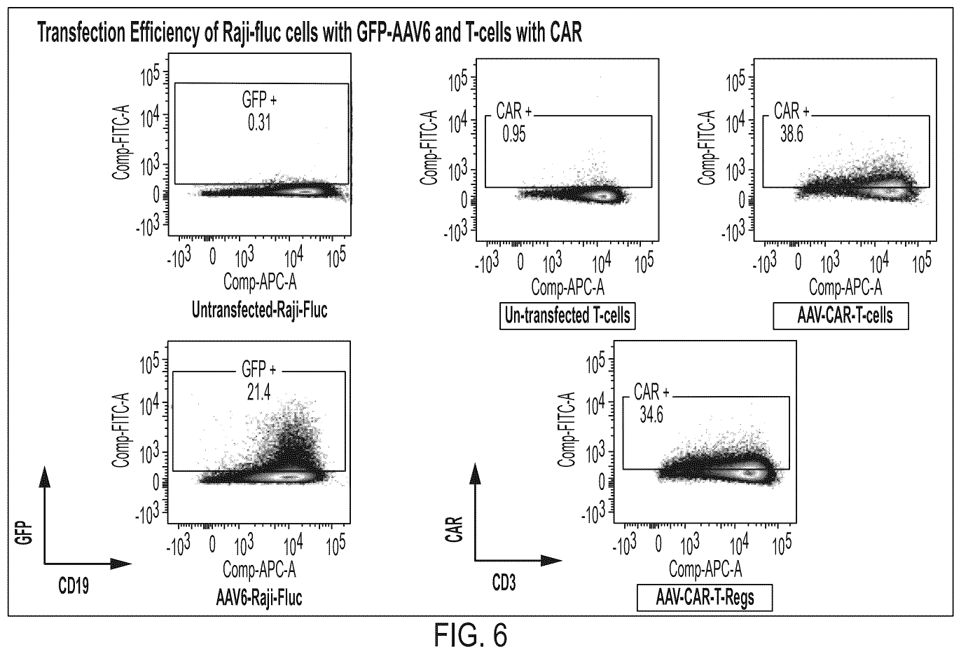

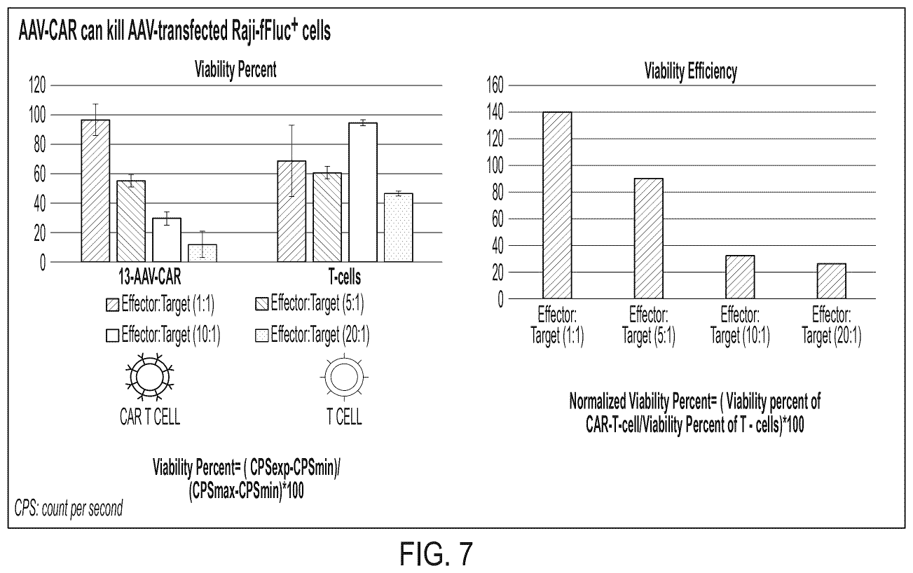

Efficacy of CAR and CAR-Treg constructs has been investigated using luciferase-based Killing and Inhibition of Killing assays. In brief, the cytotoxicity of CARs is determined by a luciferase readout, as well as the inhibition of cytotoxicity of the CAR T-regs. Raji cells constitutively express luciferase and are used as target cells. For AAV specific CARs, cells are they are infected with rAAVs containing GFP so transduction efficiency of AAVs can be determined. After co-culturing target cells with CAR effector cells (for 6-24 hrs) luciferase activity is read on the plate. Decreased luciferase indicates decreased cell viability and efficient cytotoxic ability, whereas increased luciferase activity indicates increased cell viability and inhibition of cytotoxic ability. For these experiments AAV6 was used to infect the Raji cells. With increasing amounts of Effector AAV CARs (from 1:1 Effector to Target, to 1:20 Effector to Target) a dose dependent killing of the AAV infected Raji cells. This effect was not observed in untransfected T-cells cells, and when killing in normalized to untransfected T-cells dose dependent result is still observed. Representative data are shown in FIGS. 6 and 7.

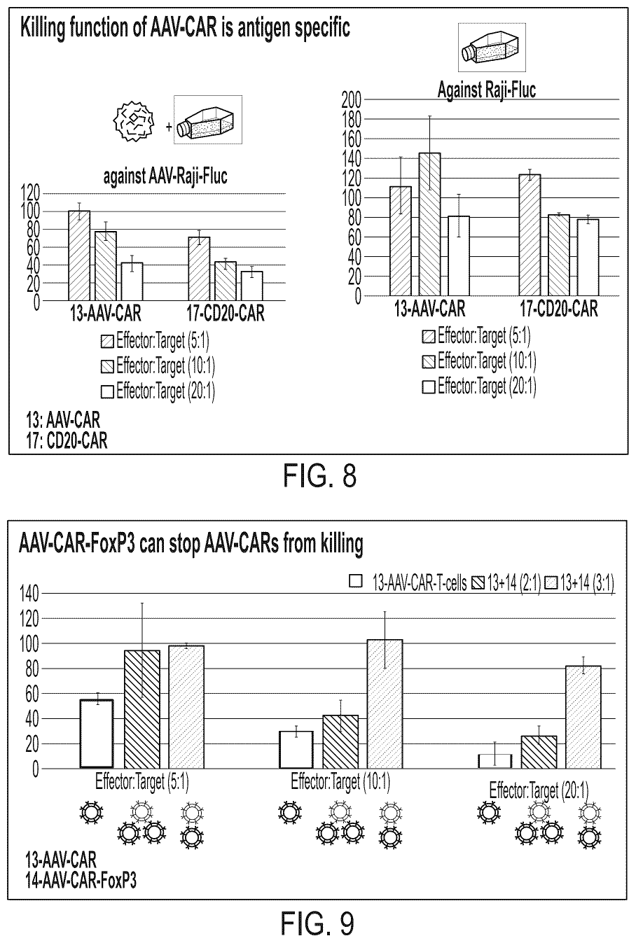

Importantly, this response is antigen specific and only observed when Raji cells are infected with AAV virus (FIG. 8). When AAV-CARs undergo the same assay but with cells that are not infected with AAV they do not display dose dependent killing. When Raji-cells are infected with AAV, dose dependent killing is observed.

The T-regulatory assays are performed in the same manner, except that Target cells are mixed with both CAR-AAV target cells as well as CAR-AAV T-regulatory cells. At 10:1 and 20:1 Effector to Target ratios, when CAR T-Cells and CAR T-regs are mixed at equal ratios robust inhibition of killing is observed (FIG. 9). The CAR T-reg response is able to inhibit antigen specific killing.

### Example 2

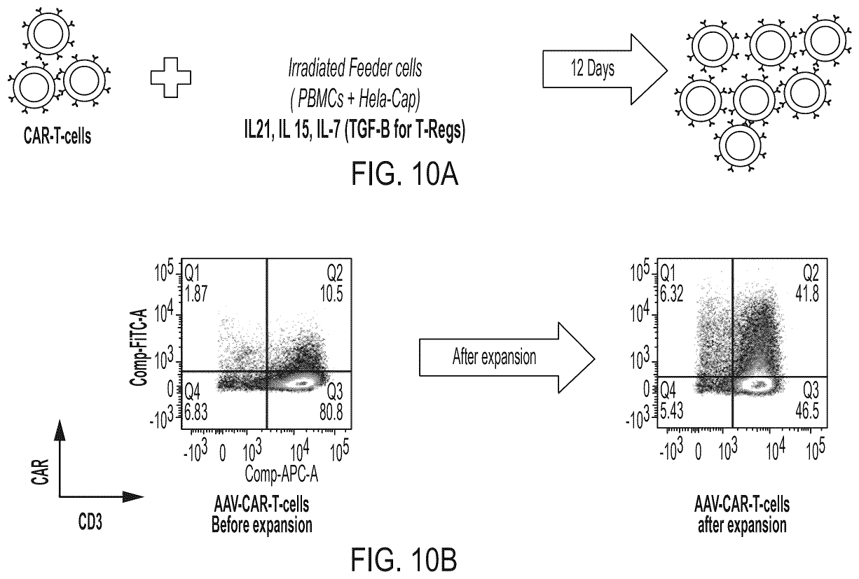

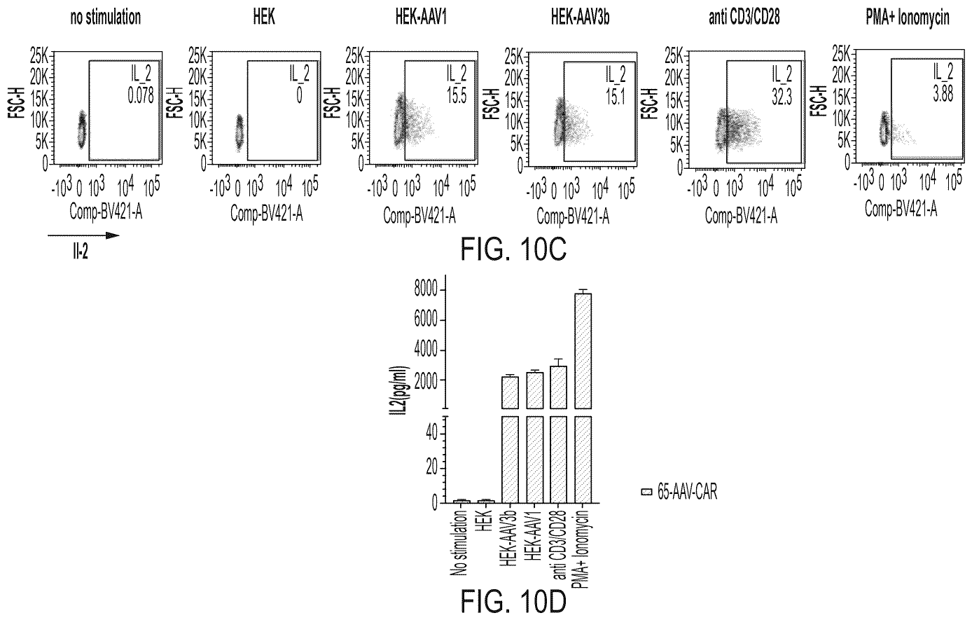

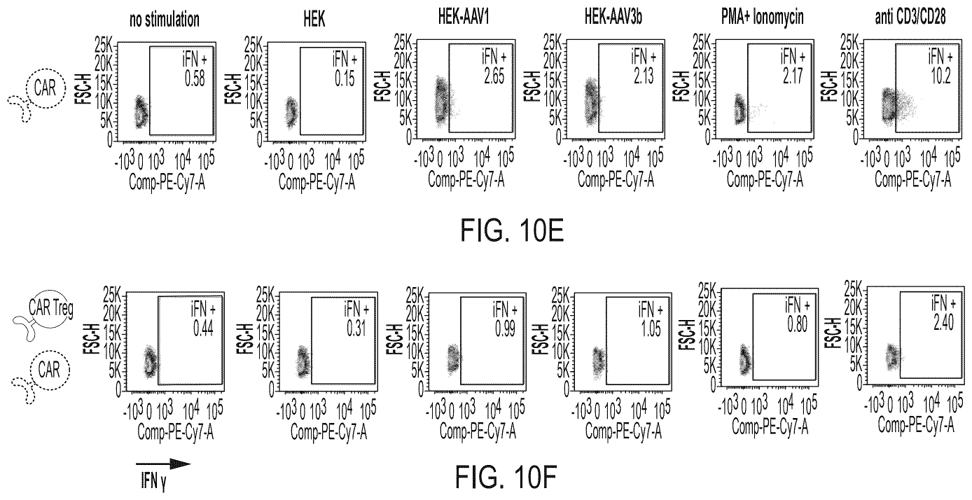

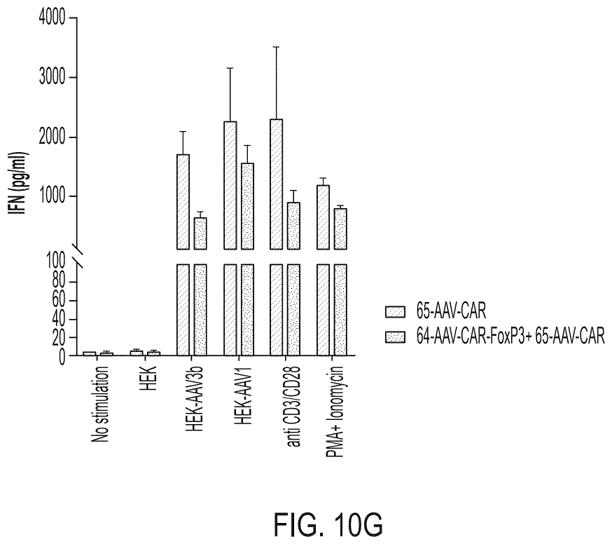

AAV-CAR-T-cells and AAV-CAR-Regulatory-T-cells were put through an expansion protocol (FIGS. 10A-10G) and measured by representative flow cytometry plots of transfected cells before and after expansion (FIG. 10B). Flow cytometry plots of IL-2 production by AAV-CAR-T-cells after different stimulation conditions (FIG. 10C). IL-2 concentration of AAV-CAR-T-cells after different stimulation conditions by ELISA (FIG. 10D). Flow cytometry plots of IFN-7 production by AAV-CAR-T-cells after different stimulation conditions (FIG. 10E). Flow cytometry plots of IFN-7 production by AAV-CAR-T-cells co-cultured with AAV-CAR-Regulatory-T-cells after different stimulation conditions (FIG. 10F). IFN-7 concentration of AAV-CAR-T-cells with or without AAV-CAR-Regulatory-T-cells after different stimulation conditions by ELISA (FIG. 10G).

### Example 3

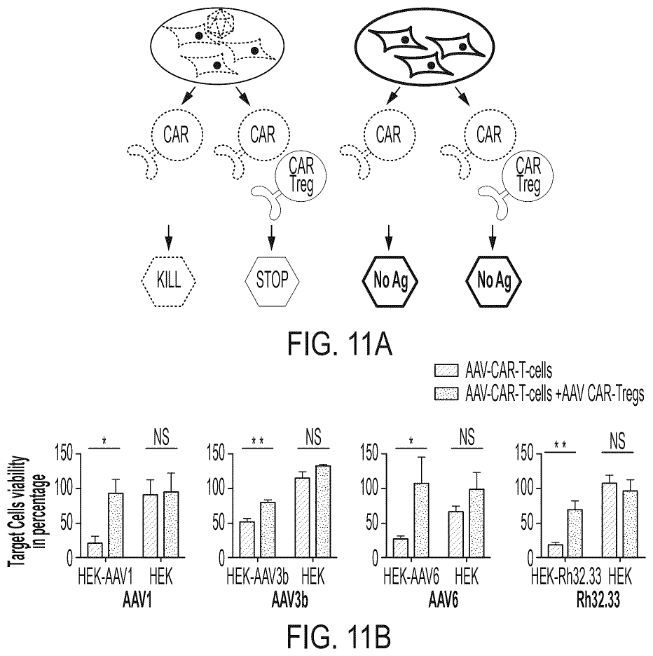

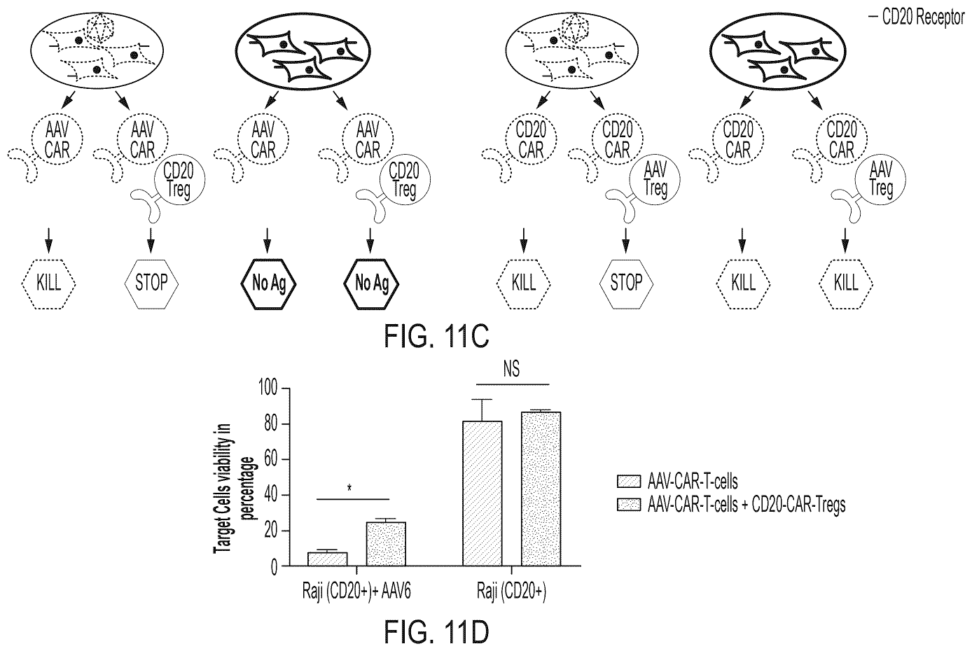

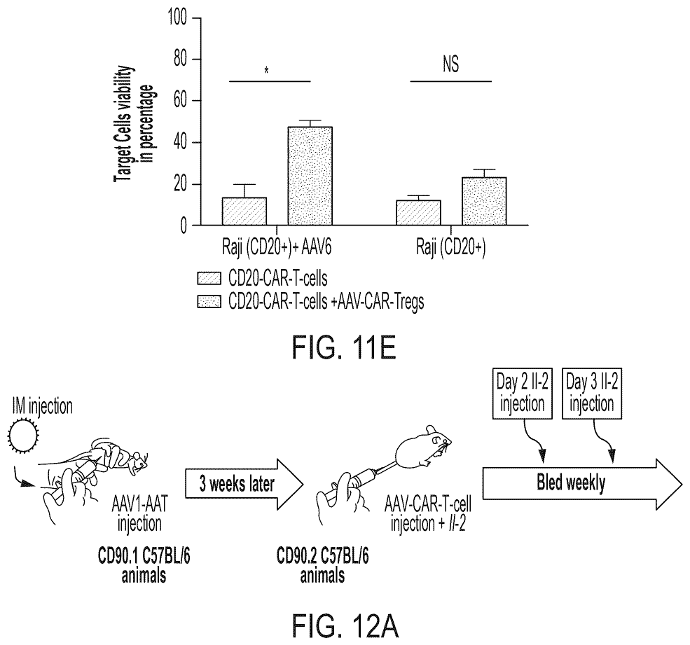

FIGS. 11A-11E. FIG. 11A: Graphical schematic of a luciferase killing assay and inhibition of luciferase killing assay. FIG. 11B: Quantitative results of killing assay and inhibition of killing assay for AAV1, AAV6, AAV3b and Rh32.33 capsid variants. FIG. 11C: Graphical schematic of a luciferase killing assay and inhibition of luciferase killing assay. FIG. 11D: Quantitative results of killing assay and inhibition of killing assay against AAV6 transfected-Raji cells (CD20+) and Raji cells (CD20+) using AAV-CAR-T-cells and CD20-CAR-Tregs. FIG. 11E: Quantitative results of killing assay and inhibition of killing assay against AAV6 transfected-Raji cells (CD20+) and Raji cells (CD20+) using CD20-CAR-T-cells and AAV-CAR-Tregs.

### Example 4

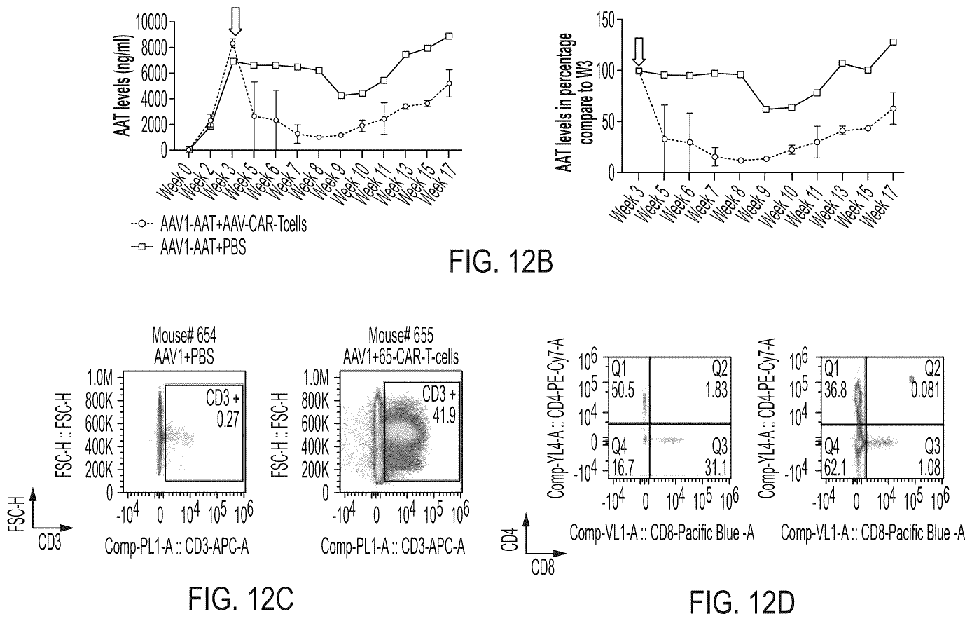

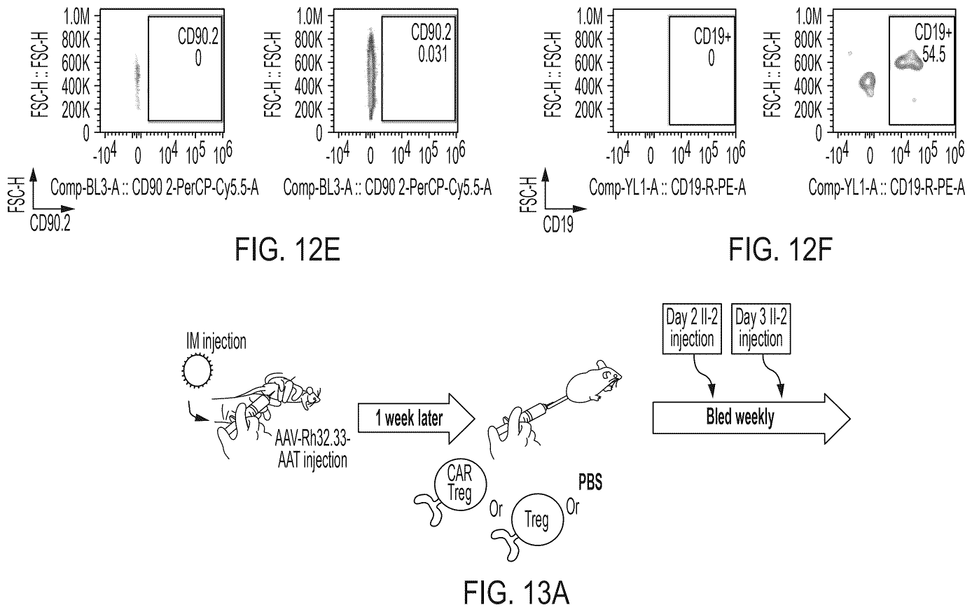

FIGS. 12A-12F. FIG. 12A: Graphical schematic of in vivo CAR-T-cell response. Intramuscular injections of AAV1-human AAT [5*10¹⁰ viral titer]. At week 3, intravenous injection of AAV-CAR-T-cells [5*10⁶ cells] or PBS followed by intraperitoneal of IL-2 [45000 IU] for 3 days. FIG. 12B: Time course of serum human AAT protein levels of control animals and animals that received AAV-CAR-T-cells (left). Normalized human AAT protein levels to the baseline at week 3 for control animals and animals that received AAV-CAR-T-cells (right). Arrows represents delivery of CAR T-cell delivery. FIGS. 12C-F: Flow cytometry plots of isolated T-cells from the muscles of AAV1-human AAT injected animals or AAV1-human AAT injected animals with AAV-CAR-T-cells. Populations of CD3+ (FIG. 12C); CD3+, CD8+, or CD4+ (FIG. 12D); CD3+, CD90.2+ (FIG. 12E); CD3+, CD90.2+, CD19+ (FIG. 12F).

### Example 5

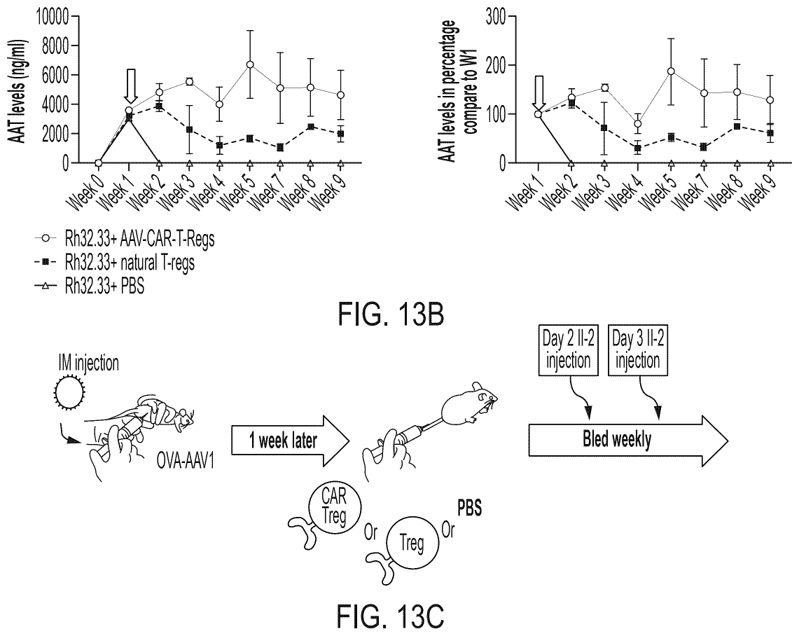

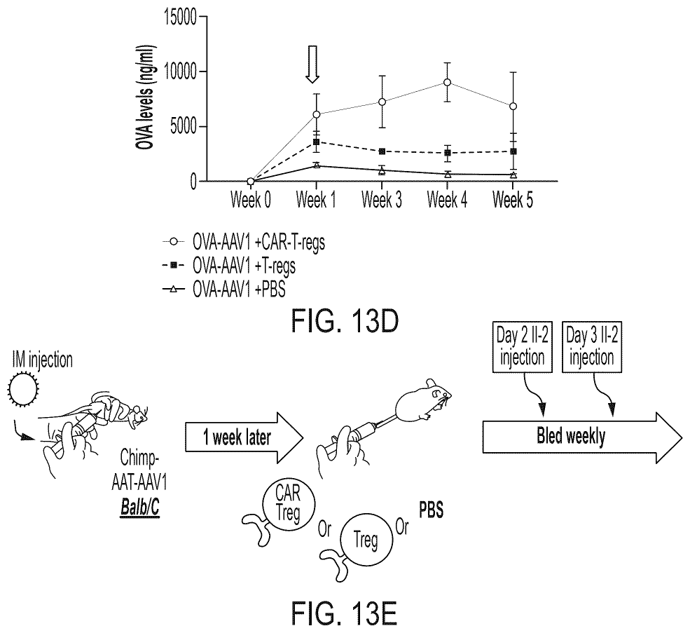

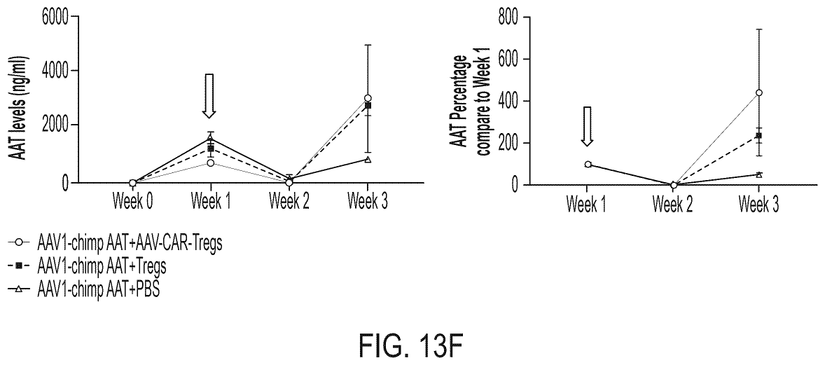

FIGS. 13A-13F. FIG. 13A: Graphical schematic of in vivo CAR-Regulatory-T-cell suppression immune response against AAV-Rh32.33. Intra muscular injections of AAV-Rh32.33-human AAT [5*1010 viral titer]. At week 1, intravenous injection of AAV-CAR-Regulatory-T-cells [5*106 cells] or natural expanded regulatory T-cells [5*106 cells] or PBS followed by Intraperitoneal of IL-2 [45000 IU] for 3 days. FIG. 13B: Time course of serum human AAT protein levels of control animals and animals that received AAV-CAR-Regulatory-T-cells and expanded natural regulatory-T-cells (left). Normalized human AAT protein levels to the baseline at week 1 for control animals and animals that received AAV-CAR-Regulatory-T-cells and expanded natural regulatory-T-cells (right). Red arrow represents CAR T-Reg delivery. FIG. 13C: Graphical schematic of in vivo CAR-Regulatory T-cell suppression immune response against Ovalbumin-AAV1 in C57BL/6 animals. Intra muscular injections of Ovalbumin-AAV1 [5*1010 viral titer]. At week 1, intravenous injection of AAV-CAR-Regulatory-T-cells [5*106 cells] or natural expanded regulatory T-cells [5*106 cells] or PBS followed intraperitoneal of IL-2 [45000 IU] for 3 days. FIG. 13D: Time course of serum Ovalbumin levels of control animals and animals that received AAV-CAR-Regulatory-T-cells and expanded natural regulatory-T-cells (left). Normalized Ovalbumin protein levels to the baseline at week 1 for control animals and animals that received AAV-CAR-Regulatory-T-cells and expanded natural regulatory-T-cells (right). Red arrow represents CAR T-Reg delivery. FIG. 13E: Graphical schematic of in vivo CAR-Regulatory-T-cell suppression immune response against chimpanzee AAT-AAV1 in Balb/C animals. Intra muscular injections of chimpanzee AAT-AAV1 [5*1010 viral titer]. At week 1, intravenous injection of of AAV-CAR-Regulatory-T-cells [5*106 cells] or natural expanded regulatory T-cells [5*106 cells] or PBS followed by intraperitoneal of IL-2 [45000 IU] for 3 days. FIG. 13F: Time course of serum chimpanzee AAT protein levels of control animals and animals that received AAV-CAR-Regulatory-T-cells and expanded natural regulatory-T-cells (left). Normalized chimpanzee AAT protein levels to the baseline at week 1 for control animals and animals that received AAV-CAR-Regulatory-T-cells and expanded natural regulatory-T-cells (right). Red arrow represents CAR T-reg delivery.

### Exemplary Sequences

This Table exhibits some exemplary sequences as disclosed by the instant Specification, but is not limiting. This Specification includes a Sequence Listing submitted concurrently herewith as a text file in ASCII format. The Sequence Listing and all of the information contained therein are expressly incorporated herein and constitute part of the instant Specification as filed.

TABLE 1
Exemplary Sequences

| SEQ ID | | |
|---|---|---|
| NO: | Sequence*-Nucleic Acid | Description |
| 1 | ctgttttaaaagaaaaggggggattggggggtacagtgcaggggaaagaatagtagacataatagcaaca | 13-1_Lenti- |
| | gacatacaaactaaagaattacaaaaacaaattacaaaaattcaaaattttatcaacaagcttgatcgatggct | IF5-D3-NQ- |
| | ccggtgcccgtcagtgggcagagcgcacatcgcccacagtccccgagaagttggggggaggggtcggc | 28-BB-Z |
| | aattgaaccggtgcctagagaaggtggcgcggggtaaactgggaaagtgatgtcgtgtactggctccgcct | |
| | ttttcccgagggtgggggagaaccgtatataagtgcagtagtcgccgtgaacgttctttttcgcaacgggtttg | |
| | ccgccagaacacaggtgtcgtgacgcggatccacgcgtgcggccgcacgcgtaagcttagcgcagagg | |
| | cttggggcagccgagcggcagccaggccccggcccgggcctcggttccagaagggagaggagcccgc | |
| | caaggcgcgcaagagagcgggctgcctcgcagtccgagccggagagggagcgcgagccgcgccggc | |
| | cccggacggcctcgccgccaccATGGAGACAGACACACTCCTGCTATGGGT | |
| | GCTGCTGCTCTGGGTTCCAGGTTCCACTGGTGACATTGTGCTGAC | |
| | ACAGTCTCCTGCTTCCTTAGCTGTATCTCTGGGGCAGAGGGCCAC | |
| | CATCTCATGCAGGGCCAGCCAAAGTGTCAGTTCATCTACCTATA | |
| | ATTTTATACACTGGTATCAACAGAAATCAGGACAGCCACCCAAA | |
| | CTCCTCATCAAGTATGCATCCAACCTAGAATCTGGGGTCCCTGTC | |
| | AGGTTCAGTGGCAGTGGGTCTGGGACAGACTTCTCCCTCAACAT | |
| | CCATCCTGTGGAGGAGGAGGATACTGCAACATATTACTGTCAGC | |
| | ACAGTTGGGAGATTCCATACACGTTCGGAGGGGGGGCCAAGTTG | |
| | GAGATAAAAggcagtactagcggtggtggctccgggggcggttccggtgggggcggcagcag | |
| | cCAGGTGCAGCTGAAGGAGTCAGGACCTGGCCTGGTGGCACCCT | |
| | CACAGAGCCTGTCCATCACATGCACCGTCTCAGGATTCTCATTAA | |
| | GCGGCTATGGCATAAGCTGGGTTCGCCAGCCTCCAGGAAAGCGT | |
| | CTGGAATGGCTGGGATTGATATGGGGTGATGGAACCACAGACTA | |
| | TAATTCAGCTCTCAAATCCAGACTGAGCATCAGCAAGGACAACT | |
| | CCAAGAGCCAGGTTTTCTTAAAAATGAACAGTCTGCAAACTGAT | |
| | GACACAGCCAGGTACTTCTGTACCAGAGGGCCTCCGGCCTTCTA | |
| | TAAGTACCTCTACTTTGACTACTGGGGCCAAGGCACCACTCTCAC | |
| | AGTCTCCTCAgacaaaactcacacatgcccaccgtgcccagcacctcctgtggcaggaccgtca | |
| | gtcttcctcttccccccaaaacccaaggacaccctcatgatctcccggacccctgaggtcacatgcgtggtg | |
| | gtggacgtgagccacgaagaccctgaggtcaagttcaactggtacgtggacggcgtggaggtgcataatg | |
| | ccaagacaaagccgcgggaggagcagtaccagagcacgtaccgtgtggtcagcgtcctcaccgtcctgc | |
| | accaggactggctgaatggcaaggagtacaagtgcaaggtctccaacaaagccctcccagcccccatcga | |
| | gaaaaccatctccaaagccaaagggcagccccgagaaccacaggtgtacaccctgccaccatcacgaga | |
| | tgagctgaccaagaaccaggtcagcctgacctgcctggtcaaaggcttctatcccagcgacatcgccgtgg | |
| | agtgggagagcaatgggcagccggagaacaactacaagaccacgcctcccgtgctggactccgacggct | |
| | ccttcttcctctacagcaagctcaccgtggacaagagcaggtggcagcaggggaacgtcttctcatgctccg | |
| | tgatgcatgaggctctgcacaaccactacacgcagaagagcctctccctgtctcccttttgggtgctggtggt | |
| | ggttggtggagtcctggcttgctatagcttgctagtaacagtggcctttattattttctgggtgaggagtaagag | |
| | gagcaggggaggtcacagtgactacatgaacatgactccccgccgccccgggcccacccgcaagcatta | |
| | ccagccctatgccccaccacgcgacttcgcagcctatcgctccaaacggggcagaaagaaactcctgtata | |
| | tattcaaacaaccatttatgagaccagtacaaactactcaagaggaagatggctgtagctgccgatttccaga | |
| | agaagaagaaggaggatgtgaactgagagtgaagttcagcaggagcgcagacgcccccgcgtaccagc | |
| | agggccagaaccagctctataacgagctcaatctaggacgaagagaggagtacgatgttttggacaagag | |
| | acgtggccgggaccctgagatggggggaaagccgagaaggaagaaccctcaggaaggcctgtacaatg | |
| | aactgcagaaagataagatggcggaggcctacagtgagattgggatgaaaggcgagcgccggaggggc | |
| | aaggggcacgatggcctttaccagggtctcagtacagccaccaaggacacctacgacgcccttcacatgc | |
| | aggccctgccccctcgctgagatatcgtgggaattcgagcatcttaccgccatttattcccatatttgttctgatt | |
| | tcttgatttgggtatacatttaaatgttaataaaacaaaatggtggggcaatcatttacatttttagggatatgtaat | |
| | tactagttcaggtgtattgccacaagacaaacatgttaagaaactttcccgttatttacgctctgttcctgttaatc | |
| | aacctctggattacaaaatttgtgaaagattgactgatattcttaactatgttgctccttttacgctgtgtggatatg | |
| | ctgctttatagcctctgtatctagctattgcttcccgtacggctttcgttttctcctccttgtataaatcctggttgct | |
| | gtctcttttagaggagttgtggcccgttgtccgtcaacgtggcgtggtgtgctctgtgtttgctgacgcaaccc | |
| | ccactggctggggcattgccaccacctgtcaactcctttctgggactttcgctttccccctcccgatcgccacg | |
| | gcagaactcatcgccgcctgccttgcccgctgctggacaggggctaggttgctgggcactgataattccgt | |
| | ggtgttgtcggggaagctgacgtcctttcctcgagggggggcccggtacctttaagaccaatgacttacaag | |
| | gcagctgtagatcttagccactttttaaaagaaaaggggggactggaagggctaattcactcccaacgaaga | |
| | caagatctgctttttgcttgtactgggtctctctggttagaccagatctgagcctgggagctctctggctaacta | |
| | gggaacccactgcttaagcctcaataaagcttgccttgagtgcttcaagtagtgtgtgcccgtctgttgtgtga | |
| | ctctggtaactagagatccctcagacccttttagtcagtgtggaaaatctctagcagtagtagttcatgtcatctt | |
| | attattcagtatttataacttgcaaagaaatgaatatcagagagtgagaggaacttgtttattgcagcttataatg | |
| | gttacaaataaagcaatagcatcacaaatttcacaaataaagcattatttcactgcattctagttgtggtttgtcc | |
| | aaactcatcaatgtatcttatcatgtctggctctagctatcccgcccctaactccgcccagttccgcccattctc | |
| | cgccccatggctgactaattttttttatttatgcagaggccgaggccgcctcggcctctgagctattccagaag | |
| | tagtgaggaggcttttttggaggcctaggcttttgcgtcgagacgtacccaattcgccctatagtgagtcgtatt | |
| | acgcgcgctcactggccgtcgttttacaacgtcgtgactgggaaaaccctggcgttacccaacttaatcgcct | |
| | tgcagcacatccccctttcgccagctggcgtaatagcgaagaggcccgcaccgatcgcccttcccaacagt | |
| | tgcgcagcctgaatggcgaatggcgcgacgcgccctgtagcggcgcattaagcgcggcgggtgtggtgg | |
| | ttacgcgcagcgtgaccgctacacttgccagcgccctagcgcccgctcctttcgctttcttcccttcctttctcg | |
| | ccacgttcgccggctttccccgtcaagctctaaatcgggggctccctttagggttccgatttagtgctttacgg | |
| | cacctcgaccccaaaaaacttgattagggtgatggttcacgtagtgggccatcgccctgatagacggtttttc | |
| | gccctttgacgttggagtccacgttctttaatagtggactcttgttccaaactggaacaacactcaaccctatct | |
| | cggtctattcttttgatttataagggattttgccgatttcggcctattggttaaaaaatgagctgatttaacaaaaat | |
| | ttaacgcgaattttaacaaaatattaacgtttacaatttcccaggtggcacttttcggggaaatgtgcgcggaac | |
| | ccctatttgtttatttttctaaatacattcaaatatgtatccgctcatgagacaataaccctgataaatgcttcaata | |
| | atattgaaaaaggaagagtatgagtattcaacatttccgtgtcgcccttattcccttttttgcggcattttgccttc | |
| | ctgtttttgctcacccagaaacgctggtgaaagtaaaagatgctgaagatcagttgggtgcacgagtgggtta | |
| | catcgaactggatctcaacagcggtaagatccttgagagttttcgccccgaagaacgttttccaatgatgagc | |
| | acttttaaagttctgctatgtggcgcggtattatcccgtattgacgccgggcaagagcaactcggtcgccgca | |
| | tacactattctcagaatgacttggttgagtactcaccagtcacagaaaagcatcttacggatggcatgacagta | |
| | agagaattatgcagtgctgccataaccatgagtgataacactgcggccaacttacttctgacaacgatcgga | |
| | ggaccgaaggagctaaccgcttttttgcacaacatgggggatcatgtaactcgccttgatcgttgggaaccg | |
| | gagctgaatgaagccataccaaacgacgagcgtgacaccacgatgcctgtagcaatggcaacaacgttgc | |
| | gcaaactattaactggcgaactacttactctagcttcccggcaacaattaatagactggatggaggcggataa | |
| | agttgcaggaccacttctgcgctcggcccttccggctggctggtttattgctgataaatctggagccggtgag | |
| | cgtgggtctcgcggtatcattgcagcactggggccagatggtaagccctcccgtatcgtagttatctacacg | |
| | acggggagtcaggcaactatggatgaacgaaatagacagatcgctgagataggtgcctcactgattaagca | |
| | ttggtaactgtcagaccaagtttactcatatatactttagattgatttaaaacttcatttttaatttaaaaggatctag | |
| | gtgaagatcctttttgataatctcatgaccaaaatcccttaacgtgagttttcgttccactgagcgtcagacccc | |
| | gtagaaaagatcaaaggatcttcttgagatcctttttttctgcgcgtaatctgctgcttgcaaacaaaaaaacca | |
| | ccgctaccagcggtggtttgtttgccggatcaagagctaccaactctttttccgaaggtaactggcttcagca | |
| | gagcgcagataccaaatactgtccttctagtgtagccgtagttaggccaccacttcaagaactctgtagcacc | |
| | gcctacatacctcgctctgctaatcctgttaccagtggctgctgccagtggcgataagtcgtgtcttaccgggt | |
| | tggactcaagacgatagttaccggataaggcgcagcggtcgggctgaacggggggttcgtgcacacagc | |
| | ccagcttggagcgaacgacctacaccgaactgagatacctacagcgtgagctatgagaaagcgccacgct | |
| | tcccgaagggagaaaggcggacaggtatccggtaagcggcagggtcggaacaggagagcgcacgagg | |
| | gagcttccagggggaaacgcctggtatctttatagtcctgtcgggtttcgccacctctgacttgagcgtcgatt | |
| | tttgtgatgctcgtcaggggggcggagcctatggaaaaacgccagcaacgcggcctttttacggttcctggc | |
| | cttttgctggccttttgctcacatgttctttcctgcgttatcccctgattctgtggataaccgtattaccgcctttga | |
| | gtgagctgataccgctcgccgcagccgaacgaccgagcgcagcgagtcagtgagcgaggaagcggaa | |
| | gagcgcccaatacgcaaaccgcctctccccgcgcgttggccgattcattaatgcagctggcacgacaggtt | |
| | tcccgactggaaagcgggcagtgagcgcaacgcaattaatgtgagttagctcactcattaggcaccccagg | |
| | ctttacactttatgcttccggctcgtatgttgtgtggaattgtgagcggataacaatttcacacaggaaacagct | |
| | atgaccatgattacgccaagcgcgcaattaaccctcactaaagggaacaaaagctggagctgcaagcttaa | |
| | tgtagtcttatgcaatactcttgtagtcttgcaacatggtaacgatgagttagcaacatgccttacaaggagag | |
| | aaaaagcaccgtgcatgccgattggtggaagtaaggtggtacgatcgtgccttattaggaaggcaacagac | |
| | gggtctgacatggattggacgaaccactgaattgccgcattgcagagatattgtatttaagtgcctagctcgat | |
| | acataaacgggtctctctggttagaccagatctgagcctgggagctctctggctaactagggaacccactgc | |
| | ttaagcctcaataaagcttgccttgagtgcttcaagtagtgtgtgcccgtctgttgtgtgactctggtaactaga | |
| | gatccctcagacccttttagtcagtgtggaaaatctctagcagtggcgcccgaacagggacttgaaagcgaa | |
| | agggaaaccagaggagctctctcgacgcaggactcggcttgctgaagcgcgcacggcaagaggcgagg | |
| | ggcggcgactggtgagtacgccaaaaattttgactagcggaggctagaaggagagagatgggtgcgaga | |
| | gcgtcagtattaagcgggggagaattagatcgcgatgggaaaaaattcggttaaggccagggggaaagaa | |
| | aaaatataaattaaaacatatagtatgggcaagcagggagctagaacgattcgcagttaatcctggcctgtta | |
| | gaaacatcagaaggctgtagacaaatactgggacagctacaaccatcccttcagacaggatcagaagaact | |
| | tagatcattatataatacagtagcaaccctctattgtgtgcatcaaaggatagagataaaagacaccaaggaa | |
| | gctttagacaagatagaggaagagcaaaacaaaagtaagaccaccgcacagcaagcggccgctgatcttc | |
| | agacctggaggaggagatatgagggacaattggagaagtgaattatataaatataaagtagtaaaaattgaa | |
| | ccattaggagtagcacccaccaaggcaaagagaagagtggtgcagagagaaaaaagagcagtgggaat | |
| | aggagctttgttccttgggttcttgggagcagcaggaagcactatgggcgcagcctcaatgacgctgacggt | |
| | acaggccagacaattattgtctggtatagtgcagcagcagaacaatttgctgagggctattgaggcgcaaca | |
| | gcatctgttgcaactcacagtctggggcatcaagcagctccaggcaagaatcctggctgtggaaagatacct | |
| | aaaggatcaacagctcctggggatttggggttgctctggaaaactcatttgcaccactgctgtgccttggaat | |
| | gctagttggagtaataaatctctggaacagatttggaatcacacgacctggatggagtgggacagagaaatt | |
| | aacaattacacaagcttaatacactccttaattgaagaatcgcaaaaccagcaagaaaagaatgaacaagaa | |
| | ttattggaattagataaatgggcaagtttgtggaattggtttaacataacaaattggctgtggtatataaaattatt | |
| | cataatgatagtaggaggcttggtaggtttaagaatagtttttgctgtactttctatagtgaatagagttaggcag | |
| | ggatattcaccattatcgtttcagacccacctcccaaccccgaggggacccgacaggcccgaaggaatag | |
| | aagaagaaggtggagagagagacagagacagatccattcgattagtgaacggatctcgacggtatcggtt | |
| 2 | ctgttttaaaagaaaaggggggattggggggtacagtgcaggggaaagaatagtagacataatagcaaca | 14-2_Lenti- |
| | gacatacaaactaaagaattacaaaaacaaattacaaaaattcaaaattttatcaacaagcttgatcgatggct | IF5-D3-NQ- |
| | ccggtgcccgtcagtgggcagagcgcacatcgcccacagtccccgagaagttggggggaggggtcggc | 28-BB-Z |
| | aattgaaccggtgcctagagaaggtggcgcggggtaaactgggaaagtgatgtcgtgtactggctccgcct | |
| | ttttcccgagggtgggggagaaccgtatataagtgcagtagtcgccgtgaacgttctttttcgcaacgggtttg | |
| | ccgccagaacacaggtgtcgtgacgcggatccacgcgtgcggccgcacgcgtaagcttagcgcagagg | |
| | cttggggcagccgagcggcagccaggccccggcccgggcctcggttccagaagggagaggagcccgc | |
| | caaggcgcgcaagagagcgggctgcctcgcagtccgagccggagagggagcgcgagccgcgccggc | |
| | cccggacggcctcgccgccaccATGGAGACAGACACACTCCTGCTATGGGT | |
| | GCTGCTGCTCTGGGTTCCAGGTTCCACTGGTGACATTGTGCTGAC | |
| | ACAGTCTCCTGCTTCCTTAGCTGTATCTCTGGGGCAGAGGGCCAC | |
| | CATCTCATGCAGGGCCAGCCAAAGTGTCAGTTCATCTACCTATA | |
| | ATTTTATACACTGGTATCAACAGAAATCAGGACAGCCACCCAAA | |
| | CTCCTCATCAAGTATGCATCCAACCTAGAATCTGGGGTCCCTGTC | |
| | AGGTTCAGTGGCAGTGGGTCTGGGACAGACTTCTCCCTCAACAT | |
| | CCATCCTGTGGAGGAGGAGGATACTGCAACATATTACTGTCAGC | |
| | ACAGTTGGGAGATTCCATACACGTTCGGAGGGGGGGCCAAGTTG | |
| | GAGATAAAAggcagtactagcggtggtggctccgggggcggttccggtgggggcggcagcag | |
| | cCAGGTGCAGCTGAAGGAGTCAGGACCTGGCCTGGTGGCACCCT | |
| | CACAGAGCCTGTCCATCACATGCACCGTCTCAGGATTCTCATTAA | |
| | GCGGCTATGGCATAAGCTGGGTTCGCCAGCCTCCAGGAAAGCGT | |
| | CTGGAATGGCTGGGATTGATATGGGGTGATGGAACCACAGACTA | |
| | TAATTCAGCTCTCAAATCCAGACTGAGCATCAGCAAGGACAACT | |
| | CCAAGAGCCAGGTTTTCTTAAAAATGAACAGTCTGCAAACTGAT | |
| | GACACAGCCAGGTACTTCTGTACCAGAGGGCCTCCGGCCTTCTA | |
| | TAAGTACCTCTACTTTGACTACTGGGGCCAAGGCACCACTCTCAC | |
| | AGTCTCCTCAgacaaaactcacacatgcccaccgtgcccagcacctcctgtggcaggaccgtca | |
| | gtcttcctcttccccccaaaacccaaggacaccctcatgatctcccggacccctgaggtcacatgcgtggtg | |
| | gtggacgtgagccacgaagaccctgaggtcaagttcaactggtacgtggacggcgtggaggtgcataatg | |
| | ccaagacaaagccgcgggaggagcagtaccagagcacgtaccgtgtggtcagcgtcctcaccgtcctgc | |
| | accaggactggctgaatggcaaggagtacaagtgcaaggtctccaacaaagccctcccagcccccatcga | |
| | gaaaaccatctccaaagccaaagggcagccccgagaaccacaggtgtacaccctgccaccatcacgaga | |
| | tgagctgaccaagaaccaggtcagcctgacctgcctggtcaaaggcttctatcccagcgacatcgccgtgg | |
| | agtgggagagcaatgggcagccggagaacaactacaagaccacgcctcccgtgctggactccgacggct | |
| | ccttcttcctctacagcaagctcaccgtggacaagagcaggtggcagcaggggaacgtcttctcatgctccg | |
| | tgatgcatgaggctctgcacaaccactacacgcagaagagcctctccctgtctcccttttgggtgctggtggt | |
| | ggttggtggagtcctggcttgctatagcttgctagtaacagtggcctttattattttctgggtgaggagtaagag | |
| | gagcaggggaggtcacagtgactacatgaacatgactccccgccgccccgggcccacccgcaagcatta | |
| | ccagccctatgccccaccacgcgacttcgcagcctatcgctccaaacggggcagaaagaaactcctgtata | |
| | tattcaaacaaccatttatgagaccagtacaaactactcaagaggaagatggctgtagctgccgatttccaga | |
| | agaagaagaaggaggatgtgaactgagagtgaagttcagcaggagcgcagacgcccccgcgtaccagc | |
| | agggccagaaccagctctataacgagctcaatctaggacgaagagaggagtacgatgttttggacaagag | |
| | acgtggccgggaccctgagatggggggaaagccgagaaggaagaaccctcaggaaggcctgtacaatg | |
| | aactgcagaaagataagatggcggaggcctacagtgagattgggatgaaaggcgagcgccggaggggc | |
| | aaggggcacgatggcctttaccagggtctcagtacagccaccaaggacacctacgacgcccttcacatgc | |
| | aggccctgccccctcgccagtgtactaattatgctctcttgaaattggctggagatgttgagagcaaccccgg | |
| | gccgATGCCCAACCCTAGGCCAGCCAAGCCTATGGCTCCTTCCTTG | |
| | GCCCTTGGCCCATCCCCAGGAGTCTTGCCAAGCTGGAAGACTGC | |
| | ACCCAAGGGCTCAGAACTTCTAGGGACCAGGGGCTCTGGGGGAC | |
| | CCTTCCAAGGTCGGGACCTGCGAAGTGGGGCCCACACCTCTTCTT | |
| | CCTTGAACCCCCTGCCACCATCCCAGCTGCAGCTGCCTACAGTGC | |
| | CCCTAGTCATGGTGGCACCGTCTGGGGCCCGACTAGGTCCCTCA | |
| | CCCCACCTACAGGCCCTTCTCCAGGACAGACCACACTTCATGCAT | |
| | CAGCTCTCCACTGTGGATGCCCATGCCCAGACCCCTGTGCTCCAA | |
| | GTGCGTCCACTGGACAACCCAGCCATGATCAGCCTCCCACCACC | |
| | TTCTGCTGCCACTGGGGTCTTCTCCCTCAAGGCCCGGCCTGGCCT | |
| | GCCACCTGGGATCAATGTGGCCAGTCTGGAATGGGTGTCCAGGG | |
| | AGCCAGCTCTACTCTGCACCTTCCCACGCTCGGGTACACCCAGG | |
| | AAAGACAGCAACCTTTTGGCTGCACCCCAAGGATCCTACCCACT | |
| | GCTGGCAAATGGAGTCTGCAAGTGGCCTGGTTGTGAGAAGGTCT | |
| | TCGAGGAGCCAGAAGAGTTTCTCAAGCACTGCCAAGCAGATCAT | |
| | CTCCTGGATGAGAAAGGCAAGGCCCAGTGCCTCCTCCAGAGAGA | |
| | AGTGGTGCAGTCTCTGGAGCAGCAGCTGGAGCTGGAAAAGGAG | |
| | AAGCTGGGAGCTATGCAGGCCCACCTGGCTGGGAAGATGGCGCT | |
| | GGCCAAGGCTCCATCTGTGGCCTCAATGGACAAGAGCTCTTGCT | |
| | GCATCGTAGCCACCAGTACTCAGGGCAGTGTGCTCCCGGCCTGG | |
| | TCTGCTCCTCGGGAGGCTCCAGACGGCGGCCTGTTTGCAGTGCG | |
| | GAGGCACCTCTGGGGAAGCCATGGCAATAGTTCCTTCCCAGAGT | |
| | TCTTCCACAACATGGACTACTTCAAGTACCACAATATGCGACCCC | |
| | CTTTCACCTATGCCACCCTTATCCGATGGGCCATCCTGGAAGCCC | |
| | CGGAGAGGCAGAGGACACTCAATGAAATCTACCATTGGTTTACT | |
| | CGCATGTTCGCCTACTTCAGAAACCACCCCGCCACCTGGAAGAA | |
| | TGCCATCCGCCACAACCTGAGCCTGCACAAGTGCTTTGTGCGAG | |
| | TGGAGAGCGAGAAGGGAGCAGTGTGGACCGTAGATGAATTTGA | |
| | GTTTCGCAAGAAGAGGAGCCAACGCCCCAACAAGTGCTCCAATC | |
| | CCTGCCCTTGAaattcgagcatcttaccgccatttattcccatatttgttctgtttttcttgatttgggtat | |
| | acatttaaatgttaataaaacaaaatggtggggcaatcatttacatttttagggatatgtaattactagttcaggtg | |
| | tattgccacaagacaaacatgttaagaaactttcccgttatttacgctctgttcctgttaatcaacctctggattac | |
| | aaaatttgtgaaagattgactgatattcttaactatgttgctccttttacgctgtgtggatatgctgctttatagcct | |
| | ctgtatctagctattgcttcccgtacggctttcgttttctcctccttgtataaatcctggttgctgtctcttttagagg | |
| | agttgtggcccgttgtccgtcaacgtggcgtggtgtgctctgtgtttgctgacgcaacccccactggctgggg | |
| | cattgccaccacctgtcaactcctttctgggactttcgctttccccctcccgatcgccacggcagaactcatcg | |
| | ccgcctgccttgcccgctgctggacaggggctaggttgctgggcactgataattccgtggtgttgtcgggga | |
| | agctgacgtcctttcctcgagggggggcccggtacctttaagaccaatgacttacaaggcagctgtagatctt | |
| | agccactttttaaaagaaaaggggggactggaagggctaattcactcccaacgaagacaagatctgcttttt | |
| | gcttgtactgggtctctctggttagaccagatctgagcctgggagctctctggctaactagggaacccactgc | |
| | ttaagcctcaataaagcttgccttgagtgcttcaagtagtgtgtgcccgtctgttgtgtgactctggtaactaga | |
| | gatccctcagacccttttagtcagtgtggaaaatctctagcagtagtagttcatgtcatcttattattcagtatttat | |
| | aacttgcaaagaaatgaatatcagagagtgagaggaacttgtttattgcagcttataatggttacaaataaagc | |
| | aatagcatcacaaatttcacaaataaagcatttttttcactgcattctagttgtggtttgtccaaactcatcaatgta | |
| | tcttatcatgtctggctctagctatcccgcccctaactccgcccagttccgcccattctccgccccatggctga | |
| | ctaattttttttatttatgcagaggccgaggccgcctcggcctctgagctattccagaagtagtgaggaggcttt | |
| | tttggaggcctaggcttttgcgtcgagacgtacccaattcgccctatagtgagtcgtattacgcgcgctcactg | |
| | gccgtcgttttacaacgtcgtgactgggaaaaccctggcgttacccaacttaatcgccttgcagcacatcccc | |
| | ctttcgccagctggcgtaatagcgaagaggcccgcaccgatcgcccttcccaacagttgcgcagcctgaat | |
| | ggcgaatggcgcgacgcgccctgtagcggcgcattaagcgcggcgggtgtggtggttacgcgcagcgtg | |
| | accgctacacttgccagcgccctagcgcccgctcctttcgctttcttcccttcctttctcgccacgttcgccggc | |
| | tttccccgtcaagctctaaatcgggggctccctttagggttccgatttagtgctttacggcacctcgaccccaa | |
| | aaaacttgattagggtgatggttcacgtagtgggccatcgccctgatagacggtttttcgccctttgacgttgg | |
| | agtccacgttctttaatagtggactcttgttccaaactggaacaacactcaaccctatctcggtctattcttttgat | |
| | ttataagggattttgccgatttcggcctattggttaaaaaatgagctgatttaacaaaaatttaacgcgaattttaa | |
| | caaaatattaacgtttacaatttcccaggtggcacttttcggggaaatgtgcgcggaacccctatttgtttattttt | |
| | ctaaatacattcaaatatgtatccgctcatgagacaataaccctgataaatgcttcaataatattgaaaaaggaa | |
| | gagtatgagtattcaacatttccgtgtcgcccttattcccttttttgcggcattttgccttcctgtttttgctcaccca | |
| | gaaacgctggtgaaagtaaaagatgctgaagatcagttgggtgcacgagtgggttacatcgaactggatct | |
| | caacagcggtaagatccttgagagttttcgccccgaagaacgttttccaatgatgagcacttttaaagttctgct | |
| | atgtggcgcggtattatcccgtattgacgccgggcaagagcaactcggtcgccgcatacactattctcagaa | |
| | tgacttggttgagtactcaccagtcacagaaaagcatcttacggatggcatgacagtaagagaattatgcagt | |
| | gctgccataaccatgagtgataacactgcggccaacttacttctgacaacgatcggaggaccgaaggagct | |
| | aaccgcttttttgcacaacatgggggatcatgtaactcgccttgatcgttgggaaccggagctgaatgaagcc | |
| | ataccaaacgacgagcgtgacaccacgatgcctgtagcaatggcaacaacgttgcgcaaactattaactgg | |
| | cgaactacttactctagcttcccggcaacaattaatagactggatggaggcggataaagttgcaggaccactt | |
| | ctgcgctcggcccttccggctggctggtttattgctgataaatctggagccggtgagcgtgggtctcgcggta | |
| | tcattgcagcactggggccagatggtaagccctcccgtatcgtagttatctacacgacggggagtcaggca | |
| | actatggatgaacgaaatagacagatcgctgagataggtgcctcactgattaagcattggtaactgtcagac | |
| | caagtttactcatatatactttagattgatttaaaacttcatttttaatttaaaaggatctaggtgaagatcctttttga | |
| | taatctcatgaccaaaatcccttaacgtgagttttcgttccactgagcgtcagaccccgtagaaaagatcaaa | |
| | ggatcttcttgagatcctttttttctgcgcgtaatctgctgcttgcaaacaaaaaaaccaccgctaccagcggtg | |
| | gtttgtttgccggatcaagagctaccaactctttttccgaaggtaactggcttcagcagagcgcagataccaa | |
| | atactgtccttctagtgtagccgtagttaggccaccacttcaagaactctgtagcaccgcctacatacctcgct | |
| | ctgctaatcctgttaccagtggctgctgccagtggcgataagtcgtgtcttaccgggttggactcaagacgat | |
| | agttaccggataaggcgcagcggtcgggctgaacggggggttcgtgcacacagcccagcttggagcgaa | |
| | cgacctacaccgaactgagatacctacagcgtgagctatgagaaagcgccacgcttcccgaagggagaa | |
| | aggcggacaggtatccggtaagcggcagggtcggaacaggagagcgcacgagggagatccaggggg | |
| | aaacgcctggtatattatagtcctgtcgggtttcgccacctctgacttgagcgtcgatttttgtgatgacgtca | |
| | ggggggcggagcctatggaaaaacgccagcaacgcggcctttttacggttcctggccttttgctggcctttt | |
| | gacacatgttattcctgcgttatcccctgattctgtggataaccgtattaccgcctttgagtgagctgataccg | |
| | ctcgccgcagccgaacgaccgagcgcagcgagtcagtgagcgaggaagcggaagagcgcccaatacg | |
| | caaaccgcctctccccgcgcgttggccgattcattaatgcagctggcacgacaggtttcccgactggaaag | |
| | cgggcagtgagcgcaacgcaattaatgtgagttagacactcattaggcaccccaggattacactttatgat | |
| | ccggctcgtatgttgtgtggaattgtgagcggataacaatttcacacaggaaacagctatgaccatgattacg | |
| | ccaagcgcgcaattaaccacactaaagggaacaaaagctggagctgcaagataatgtagtatatgcaat | |
| | actatgtagtatgcaacatggtaacgatgagttagcaacatgccttacaaggagagaaaaagcaccgtgc | |
| | atgccgattggtggaagtaaggtggtacgatcgtgccttattaggaaggcaacagacgggtctgacatggat | |
| | tggacgaaccactgaattgccgcattgcagagatattgtatttaagtgcctagctcgatacataaacgggtac | |
| | tctggttagaccagatctgagcctgggagactctggctaactagggaacccactgcttaagcctcaataaa | |
| | gatgccttgagtgatcaagtagtgtgtgcccgtctgttgtgtgactctggtaactagagatccacagaccct | |
| | tttagtcagtgtggaaaatctctagcagtggcgcccgaacagggacttgaaagcgaaagggaaaccagag | |
| | gagactacgacgcaggactcggcttgctgaagcgcgcacggcaagaggcgaggggcggcgactggt | |
| | gagtacgccaaaaattttgactagcggaggctagaaggagagagatgggtgcgagagcgtcagtattaag | |
| | cgggggagaattagatcgcgatgggaaaaaattcggttaaggccagggggaaagaaaaaatataaattaa | |
| | aacatatagtatgggcaagcagggagctagaacgattcgcagttaatcctggcctgttagaaacatcagaag | |
| | gctgtagacaaatactgggacagctacaaccatcccttcagacaggatcagaagaacttagatcattatataa | |
| | tacagtagcaaccctctattgtgtgcatcaaaggatagagataaaagacaccaaggaagctttagacaagat | |
| | agaggaagagcaaaacaaaagtaagaccaccgcacagcaagcggccgctgatcttcagacctggagga | |
| | ggagatatgagggacaattggagaagtgaattatataaatataaagtagtaaaaattgaaccattaggagtag | |
| | cacccaccaaggcaaagagaagagtggtgcagagagaaaaaagagcagtgggaataggagctttgttcc | |
| | ttgggttcttgggagcagcaggaagcactatgggcgcagcctcaatgacgctgacggtacaggccagaca | |
| | attattgtctggtatagtgcagcagcagaacaatttgctgagggctattgaggcgcaacagcatctgttgcaa | |
| | ctcacagtctggggcatcaagcagctccaggcaagaatcctggctgtggaaagatacctaaaggatcaaca | |
| | gctcctggggatttggggttgctctggaaaactcatttgcaccactgctgtgccttggaatgctagttggagta | |
| | ataaatctctggaacagatttggaatcacacgacctggatggagtgggacagagaaattaacaattacacaa | |
| | gcttaatacactccttaattgaagaatcgcaaaaccagcaagaaaagaatgaacaagaattattggaattaga | |
| | taaatgggcaagtttgtggaattggtttaacataacaaattggctgtggtatataaaattattcataatgatagta | |
| | ggaggcttggtaggtttaagaatagtttttgctgtactttctatagtgaatagagttaggcagggatattcaccat | |
| | tatcgtttcagacccacctcccaaccccgaggggacccgacaggcccgaaggaatagaagaagaaggtg | |
| | gagagagagacagagacagatccattcgattagtgaacggatctcgacggtatcggtt | |
| 3 | ctgttttaaaagaaaaggggggattggggggtacagtgcaggggaaagaatagtagacataatagcaaca | CD8leader- |
| | gacatacaaactaaagaattacaaaaacaaattacaaaaattcaaaattttatcaacaagcttgatcgatggct | AAV-CAR- |
| | ccggtgcccgtcagtgggcagagcgcacatcgcccacagtccccgagaagttggggggaggggtcggc | 2A-EGFRt |
| | aattgaaccggtgcctagagaaggtggcgcggggtaaactgggaaagtgatgtcgtgtactggctccgcct | |
| | ttttcccgagggtgggggagaaccgtatataagtgcagtagtcgccgtgaacgttctttttcgcaacgggtttg | |
| | ccgccagaacacaggtgtcgtgacgcggatccacgcgtgcggccgcacgcgtaagcttagcgcagagg | |
| | cttggggcagccgagcggcagccaggccccggcccgggcctcggttccagaagggagaggagcccgc | |
| | caaggcgcgcaagagagcgggctgcctcgcagtccgagccggagagggagcgcgagccgcgccggc | |
| | cccggacggcctcgccgccaccATGGCTCTGCCCGTCACCGCACTGCTGCTG | |
| | CCTCTGGCTCTGCTGCTGCACGCCGCAAGACCAGACATTGTGCTG | |
| | ACACAGTCTCCTGCTTCCTTAGCTGTATCTCTGGGGCAGAGGGCC | |
| | ACCATCTCATGCAGGGCCAGCCAAAGTGTCAGTTCATCTACCTAT | |
| | AATTTTATACACTGGTATCAACAGAAATCAGGACAGCCACCCAA | |
| | ACTCCTCATCAAGTATGCATCCAACCTAGAATCTGGGGTCCCTGT | |
| | CAGGTTCAGTGGCAGTGGGTCTGGGACAGACTTCTCCCTCAACA | |
| | TCCATCCTGTGGAGGAGGAGGATACTGCAACATATTACTGTCAG | |
| | CACAGTTGGGAGATTCCATACACGTTCGGAGGGGGGGCCAAGTT | |
| | GGAGATAAAAggcagtactagcggtggtggctccgggggcggttccggtgggggcggcagca | |
| | gcCAGGTGCAGCTGAAGGAGTCAGGACCTGGCCTGGTGGCACCC | |
| | TCACAGAGCCTGTCCATCACATGCACCGTCTCAGGATTCTCATTA | |
| | AGCGGCTATGGCATAAGCTGGGTTCGCCAGCCTCCAGGAAAGCG | |
| | TCTGGAATGGCTGGGATTGATATGGGGTGATGGAACCACAGACT | |
| | ATAATTCAGCTCTCAAATCCAGACTGAGCATCAGCAAGGACAAC | |
| | TCCAAGAGCCAGGTTTTCTTAAAAATGAACAGTCTGCAAACTGA | |
| | TGACACAGCCAGGTACTTCTGTACCAGAGGGCCTCCGGCCTTCT | |
| | ATAAGTACCTCTACTTTGACTACTGGGGCCAAGGCACCACTCTCA | |
| | CAGTCTCCTCAgacaaaactcacacatgcccaccgtgcccagcacctcctgtggcaggaccgtc | |
| | agtcttcctcttccccccaaaacccaaggacaccctcatgatctcccggacccctgaggtcacatgcgtggt | |
| | ggtggacgtgagccacgaagaccctgaggtcaagttcaactggtacgtggacggcgtggaggtgcataat | |
| | gccaagacaaagccgcgggaggagcagtaccagagcacgtaccgtgtggtcagcgtcctcaccgtcctg | |
| | caccaggactggctgaatggcaaggagtacaagtgcaaggtctccaacaaagccctcccagcccccatcg | |
| | agaaaaccatctccaaagccaaagggcagccccgagaaccacaggtgtacaccctgccaccatcacgag | |
| | atgagctgaccaagaaccaggtcagcctgacctgcctggtcaaaggcttctatcccagcgacatcgccgtg | |
| | gagtgggagagcaatgggcagccggagaacaactacaagaccacgcctcccgtgctggactccgacgg | |
| | ctccttcttcctctacagcaagctcaccgtggacaagagcaggtggcagcaggggaacgtcttctcatgctc | |
| | cgtgatgcatgaggctctgcacaaccactacacgcagaagagcctctccctgtctcccttttgggtgctggtg | |
| | gtggttggtggagtcctggcttgctatagcttgctagtaacagtggcctttattattttctgggtgaggagtaag | |
| | aggagcaggggaggtcacagtgactacatgaacatgactccccgccgccccgggcccacccgcaagca | |
| | ttaccagccctatgccccaccacgcgacttcgcagcctatcgctccaaacggggcagaaagaaactcctgt | |
| | atatattcaaacaaccatttatgagaccagtacaaactactcaagaggaagatggctgtagctgccgatttcc | |
| | agaagaagaagaaggaggatgtgaactgagagtgaagttcagcaggagcgcagacgcccccgcgtacc | |
| | agcagggccagaaccagctctataacgagctcaatctaggacgaagagaggagtacgatgttttggacaa | |
| | gagacgtggccgggaccctgagatggggggaaagccgagaaggaagaaccctcaggaaggcctgtac | |
| | aatgaactgcagaaagataagatggcggaggcctacagtgagattgggatgaaaggcgagcgccggag | |
| | gggcaaggggcacgatggcctttaccagggtctcagtacagccaccaaggacacctacgacgcccttcac | |
| | atgcaggccctgccccctcgccagtgtactaattatgctctcttgaaattggctggagatgttgagagcaacc | |
| | ccgggccgatgcttctcctggtgacaagccttctgctctgtgagttaccacacccagcattcctcctgatccca | |
| | cgcaaagtgtgtaacggaataggtattggtgaatttaaagactcactctccataaatgctacgaatattaaaca | |
| | cttcaaaaactgcacctccatcagtggcgatctccacatcctgccggtggcatttaggggtgactccttcaca | |
| | catactcctcctctggatccacaggaactggatattctgaaaaccgtaaaggaaatcacagggtttttgctgat | |
| | tcaggcttggcctgaaaacaggacggacctccatgcctttgagaacctagaaatcatacgcggcaggacca | |
| | agcaacatggtcagttttctcttgcagtcgtcagcctgaacataacatccttgggattacgctccctcaaggag | |
| | ataagtgatggagatgtgataatttcaggaaacaaaaatttgtgctatgcaaatacaataaactggaaaaaact | |
| | gtttgggacctccggtcagaaaaccaaaattataagcaacagaggtgaaaacagctgcaaggccacaggc | |
| | caggtctgccatgccttgtgctcccccgagggctgctggggcccggagcccagggactgcgtctcttgcc | |
| | ggaatgtcagccgaggcagggaatgcgtggacaagtgcaaccttctggagggtgagccaagggagtttgt | |
| | ggagaactctgagtgcatacagtgccacccagagtgcctgcctcaggccatgaacatcacctgcacagga | |
| | cggggaccagacaactgtatccagtgtgcccactacattgacggcccccactgcgtcaagacctgcccgg | |
| | caggagtcatgggagaaaacaacaccctggtctggaagtacgcagacgccggccatgtgtgccacctgtg | |
| | ccatccaaactgcacctacggatgcactgggccaggtcttgaaggctgtccaacgaatgggcctaagatcc | |
| | cgtccatcgccactgggatggtgggggccctcctcttgctgctggtggtggccctggggatcggcctcttca | |
| | tgtgaggaattcgagcatcttaccgccatttattcccatatttgttctgatttcttgatttgggtatacatttaaatgt | |
| | taataaaacaaaatggtggggcaatcatttacattatagggatatgtaattactagacaggtgtattgccacaa | |
| | gacaaacatgttaagaaactttcccgttatttacgctctgttcctgttaatcaacctctggattacaaaatttgtga | |
| | aagattgactgatattcttaactatgagctccattacgctgtgtggatatgctgctttatagcctctgtatctagct | |
| | attgcacccgtacggcatcgattctcctccagtataaatcctggagctgtctcattagaggagagtggccc | |
| | gttgtccgtcaacgtggcgtggtgtgctctgtgtttgctgacgcaacccccactggctggggcattgccacca | |
| | cctgtcaactcctttctgggactttcgctttccccctcccgatcgccacggcagaactcatcgccgcctgcctt | |
| | gcccgctgctggacaggggctaggttgctgggcactgataattccgtggtgttgtcggggaagctgacgtc | |
| | ctacctcgagggggggcccggtaccataagaccaatgacttacaaggcagctgtagatcttagccactatt | |
| | aaaagaaaaggggggactggaagggctaattcactcccaacgaagacaagatctgctttttgcttgtactgg | |
| | gtctctctggttagaccagatctgagcctgggagctctctggctaactagggaacccactgcttaagcctcaa | |
| | taaagcttgccttgagtgcttcaagtagtgtgtgcccgtctgttgtgtgactctggtaactagagatccctcaga | |
| | cccattagtcagtgtggaaaatctctagcagtagtagacatgtcatcttattattcagtatttataacttgcaaag | |
| | aaatgaatatcagagagtgagaggaacttgatattgcagcttataatggttacaaataaagcaatagcatcac | |
| | aaatttcacaaataaagcattatacactgcattctagagtggatgtccaaactcatcaatgtatcttatcatgtct | |
| | ggctctagctatcccgcccctaactccgcccagttccgcccattctccgccccatggctgactaattttttttatt | |
| | tatgcagaggccgaggccgcctcggcctctgagctattccagaagtagtgaggaggcattaggaggccta | |
| | ggcattgcgtcgagacgtacccaattcgccctatagtgagtcgtattacgcgcgctcactggccgtcgatta | |
| | caacgtcgtgactgggaaaaccctggcgttacccaacttaatcgccttgcagcacatccccctttcgccagct | |
| | ggcgtaatagcgaagaggcccgcaccgatcgcccttcccaacagttgcgcagcctgaatggcgaatggc | |
| | gcgacgcgccctgtagcggcgcattaagcgcggcgggtgtggtggttacgcgcagcgtgaccgctacac | |
| | ttgccagcgccctagcgcccgctccatcgcatcacccacctactcgccacgacgccggctaccccgtca | |
| | agctctaaatcgggggctccctttagggttccgatttagtgctttacggcacctcgaccccaaaaaacttgatt | |
| | agggtgatggttcacgtagtgggccatcgccctgatagacggtttttcgccctttgacgttggagtccacgttc | |
| | ataatagtggactcagaccaaactggaacaacactcaaccctatctcggtctattcattgatttataagggatt | |
| | agccgatacggcctattggttaaaaaatgagctgatttaacaaaaatttaacgcgaatataacaaaatattaa | |
| | cgtttacaatttcccaggtggcacttttcggggaaatgtgcgcggaacccctatttgtttatttttctaaatacatt | |
| | caaatatgtatccgctcatgagacaataaccctgataaatgcttcaataatattgaaaaaggaagagtatgagt | |
| | attcaacataccgtgtcgcccttattcccattagcggcattagccacctgatttgctcacccagaaacgctg | |
| | gtgaaagtaaaagatgctgaagatcagttgggtgcacgagtgggttacatcgaactggatctcaacagcgg | |
| | taagatccttgagagttttcgccccgaagaacgttttccaatgatgagcacttttaaagttctgctatgtggcgc | |
| | ggtattatcccgtattgacgccgggcaagagcaactcggtcgccgcatacactattctcagaatgacttggtt | |
| | gagtactcaccagtcacagaaaagcatcttacggatggcatgacagtaagagaattatgcagtgctgccata | |
| | accatgagtgataacactgcggccaacttacttctgacaacgatcggaggaccgaaggagctaaccgctttt | |
| | ttgcacaacatgggggatcatgtaactcgccagatcgagggaaccggagctgaatgaagccataccaaac | |
| | gacgagcgtgacaccacgatgcctgtagcaatggcaacaacgttgcgcaaactattaactggcgaactact | |
| | tactctagatcccggcaacaattaatagactggatggaggcggataaagagcaggaccacactgcgctc | |
| | ggcccttccggctggctggtttattgctgataaatctggagccggtgagcgtgggtctcgcggtatcattgca | |
| | gcactggggccagatggtaagccctcccgtatcgtagttatctacacgacggggagtcaggcaactatgga | |
| | tgaacgaaatagacagatcgctgagataggtgcctcactgattaagcattggtaactgtcagaccaagtttac | |
| | tcatatatactttagattgatttaaaacttcatttttaatttaaaaggatctaggtgaagatcctttttgataatctcat | |
| | gaccaaaatcccttaacgtgagttttcgttccactgagcgtcagaccccgtagaaaagatcaaaggatcttctt | |
| | gagatcctttttttctgcgcgtaatctgctgcttgcaaacaaaaaaaccaccgctaccagcggtggtttgtttgc | |
| | cggatcaagagctaccaactctttttccgaaggtaactggcttcagcagagcgcagataccaaatactgtcct | |
| | tctagtgtagccgtagttaggccaccacttcaagaactctgtagcaccgcctacatacctcgctctgctaatcc | |
| | tgttaccagtggctgctgccagtggcgataagtcgtgtcttaccgggttggactcaagacgatagttaccgga | |
| | taaggcgcagcggtcgggctgaacggggggttcgtgcacacagcccagcttggagcgaacgacctacac | |
| | cgaactgagatacctacagcgtgagctatgagaaagcgccacgcttcccgaagggagaaaggcggacag | |
| | gtatccggtaagcggcagggtcggaacaggagagcgcacgagggagcttccagggggaaacgcctggt | |
| | atattatagtcctgtcgggatcgccacctctgacttgagcgtcgatattgtgatgctcgtcaggggggcgga | |
| | gcctatggaaaaacgccagcaacgcggcctttttacggttcctggccttttgctggccttttgctcacatgttctt | |
| | tcctgcgttatcccctgattctgtggataaccgtattaccgcctttgagtgagctgataccgctcgccgcagcc | |
| | gaacgaccgagcgcagcgagtcagtgagcgaggaagcggaagagcgcccaatacgcaaaccgcctct | |
| | ccccgcgcgttggccgattcattaatgcagctggcacgacaggtttcccgactggaaagcgggcagtgag | |
| | cgcaacgcaattaatgtgagttagctcactcattaggcaccccaggctttacactttatgcttccggctcgtatg | |
| | ttgtgtggaattgtgagcggataacaatttcacacaggaaacagctatgaccatgattacgccaagcgcgca | |
| | attaaccctcactaaagggaacaaaagctggagctgcaagcttaatgtagtcttatgcaatactcttgtagtctt | |
| | gcaacatggtaacgatgagttagcaacatgccttacaaggagagaaaaagcaccgtgcatgccgattggtg | |
| | gaagtaaggtggtacgatcgtgccttattaggaaggcaacagacgggtctgacatggattggacgaaccac | |
| | tgaattgccgcattgcagagatattgtatttaagtgcctagctcgatacataaacgggtctctctggttagacca | |
| | gatctgagcctgggagctctctggctaactagggaacccactgcttaagcctcaataaagcttgccttgagtg | |
| | cttcaagtagtgtgtgcccgtctgttgtgtgactctggtaactagagatccctcagacccttttagtcagtgtgg | |
| | aaaatctctagcagtggcgcccgaacagggacttgaaagcgaaagggaaaccagaggagctctctcgac | |
| | gcaggactcggcttgctgaagcgcgcacggcaagaggcgaggggcggcgactggtgagtacgccaaa | |
| | aattttgactagcggaggctagaaggagagagatgggtgcgagagcgtcagtattaagcgggggagaatt | |
| | agatcgcgatgggaaaaaattcggttaaggccagggggaaagaaaaaatataaattaaaacatatagtatg | |
| | ggcaagcagggagctagaacgattcgcagttaatcctggcctgttagaaacatcagaaggctgtagacaaa | |
| | tactgggacagctacaaccatcccttcagacaggatcagaagaacttagatcattatataatacagtagcaac | |
| | cctctattgtgtgcatcaaaggatagagataaaagacaccaaggaagctttagacaagatagaggaagagc | |
| | aaaacaaaagtaagaccaccgcacagcaagcggccgctgatcttcagacctggaggaggagatatgagg | |
| | gacaattggagaagtgaattatataaatataaagtagtaaaaattgaaccattaggagtagcacccaccaag | |
| | gcaaagagaagagtggtgcagagagaaaaaagagcagtgggaataggagctttgttccttgggttcttggg | |
| | agcagcaggaagcactatgggcgcagcctcaatgacgctgacggtacaggccagacaattattgtctggta | |
| | tagtgcagcagcagaacaatttgctgagggctattgaggcgcaacagcatctgttgcaactcacagtctggg | |
| | gcatcaagcagctccaggcaagaatcctggctgtggaaagatacctaaaggatcaacagctcctggggatt | |
| | tggggttgctctggaaaactcatttgcaccactgctgtgccttggaatgctagttggagtaataaatctctgga | |
| | acagatttggaatcacacgacctggatggagtgggacagagaaattaacaattacacaagcttaatacactc | |
| | cttaattgaagaatcgcaaaaccagcaagaaaagaatgaacaagaattattggaattagataaatgggcaag | |
| | tttgtggaattggtttaacataacaaattggctgtggtatataaaattattcataatgatagtaggaggcttggta | |
| | ggtttaagaatagtttttgctgtactttctatagtgaatagagttaggcagggatattcaccattatcgtttcagac | |
| | ccacctcccaaccccgaggggacccgacaggcccgaaggaatagaagaagaaggtggagagagagac | |
| | agagacagatccattcgattagtgaacggatctcgacggtatcggtt | |
| 4 | ctgttttaaaagaaaaggggggattggggggtacagtgcaggggaaagaatagtagacataatagcaaca | AAV-CAR- |
| | gacatacaaactaaagaattacaaaaacaaattacaaaaattcaaaattttatcaacaagcttgatcgatggct | FoxP3-EGFRt |
| | ccggtgcccgtcagtgggcagagcgcacatcgcccacagtccccgagaagttggggggaggggtcggc | |
| | aattgaaccggtgcctagagaaggtggcgcggggtaaactgggaaagtgatgtcgtgtactggctccgcct | |
| | ttttcccgagggtgggggagaaccgtatataagtgcagtagtcgccgtgaacgttctttttcgcaacgggtttg | |
| | ccgccagaacacaggtgtcgtgacgcggatccacgcgtgcggccgcacgcgtaagcttagcgcagagg | |
| | cttggggcagccgagcggcagccaggccccggcccgggcctcggttccagaagggagaggagcccgc | |
| | caaggcgcgcaagagagcgggctgcctcgcagtccgagccggagagggagcgcgagccgcgccggc | |
| | cccggacggcctcgccgccaccATGGCTCTGCCCGTCACCGCACTGCTGCTG | |
| | CCTCTGGCTCTGCTGCTGCACGCCGCAAGACCAGACATTGTGCTG | |
| | ACACAGTCTCCTGCTTCCTTAGCTGTATCTCTGGGGCAGAGGGCC | |
| | ACCATCTCATGCAGGGCCAGCCAAAGTGTCAGTTCATCTACCTAT | |
| | AATTTTATACACTGGTATCAACAGAAATCAGGACAGCCACCCAA | |
| | ACTCCTCATCAAGTATGCATCCAACCTAGAATCTGGGGTCCCTGT | |
| | CAGGTTCAGTGGCAGTGGGTCTGGGACAGACTTCTCCCTCAACA | |
| | TCCATCCTGTGGAGGAGGAGGATACTGCAACATATTACTGTCAG | |
| | CACAGTTGGGAGATTCCATACACGTTCGGAGGGGGGGCCAAGTT | |
| | GGAGATAAAAggcagtactagcggtggtggctccgggggcggttccggtgggggcggcagca | |
| | gcCAGGTGCAGCTGAAGGAGTCAGGACCTGGCCTGGTGGCACCC | |
| | TCACAGAGCCTGTCCATCACATGCACCGTCTCAGGATTCTCATTA | |
| | AGCGGCTATGGCATAAGCTGGGTTCGCCAGCCTCCAGGAAAGCG | |
| | TCTGGAATGGCTGGGATTGATATGGGGTGATGGAACCACAGACT | |
| | ATAATTCAGCTCTCAAATCCAGACTGAGCATCAGCAAGGACAAC | |
| | TCCAAGAGCCAGGTTTTCTTAAAAATGAACAGTCTGCAAACTGA | |
| | TGACACAGCCAGGTACTTCTGTACCAGAGGGCCTCCGGCCTTCT | |
| | ATAAGTACCTCTACTTTGACTACTGGGGCCAAGGCACCACTCTCA | |
| | CAGTCTCCTCAgacaaaactcacacatgcccaccgtgcccagcacctcctgtggcaggaccgtc | |
| | agtcttcctcttccccccaaaacccaaggacaccctcatgatctcccggacccctgaggtcacatgcgtggt | |
| | ggtggacgtgagccacgaagaccctgaggtcaagttcaactggtacgtggacggcgtggaggtgcataat | |
| | gccaagacaaagccgcgggaggagcagtaccagagcacgtaccgtgtggtcagcgtcctcaccgtcctg | |
| | caccaggactggctgaatggcaaggagtacaagtgcaaggtctccaacaaagccctcccagcccccatcg | |
| | agaaaaccatctccaaagccaaagggcagccccgagaaccacaggtgtacaccctgccaccatcacgag | |
| | atgagctgaccaagaaccaggtcagcctgacctgcctggtcaaaggcttctatcccagcgacatcgccgtg | |
| | gagtgggagagcaatgggcagccggagaacaactacaagaccacgcctcccgtgctggactccgacgg | |
| | ctccttcttcctctacagcaagctcaccgtggacaagagcaggtggcagcaggggaacgtcttctcatgctc | |
| | cgtgatgcatgaggctctgcacaaccactacacgcagaagagcctctccctgtctcccttttgggtgctggtg | |
| | gtggttggtggagtcctggcttgctatagcttgctagtaacagtggcctttattattttctgggtgaggagtaag | |
| | aggagcaggggaggtcacagtgactacatgaacatgactccccgccgccccgggcccacccgcaagca | |
| | ttaccagccctatgccccaccacgcgacttcgcagcctatcgctccaaacggggcagaaagaaactcctgt | |
| | atatattcaaacaaccatttatgagaccagtacaaactactcaagaggaagatggctgtagctgccgatttcc | |
| | agaagaagaagaaggaggatgtgaactgagagtgaagttcagcaggagcgcagacgcccccgcgtacc | |
| | agcagggccagaaccagctctataacgagctcaatctaggacgaagagaggagtacgatgttttggacaa | |
| | gagacgtggccgggaccctgagatggggggaaagccgagaaggaagaaccctcaggaaggcctgtac | |
| | aatgaactgcagaaagataagatggcggaggcctacagtgagattgggatgaaaggcgagcgccggag | |
| | gggcaaggggcacgatggcctttaccagggtctcagtacagccaccaaggacacctacgacgcccttcac | |
| | atgcaggccctgccccctcgccagtgtactaattatgctctcttgaaattggctggagatgttgagagcaacc | |
| | ccgggccgGAATTCATGCCCAACCCTAGGCCAGCCAAGCCTATGGCT | |
| | CCTTCCTTGGCCCTTGGCCCATCCCCAGGAGTCTTGCCAAGCTGG | |
| | AAGACTGCACCCAAGGGCTCAGAACTTCTAGGGACCAGGGGCTC | |
| | TGGGGGACCCTTCCAAGGTCGGGACCTGCGAAGTGGGGCCCACA | |
| | CCTCTTCTTCCTTGAACCCCCTGCCACCATCCCAGCTGCAGCTGC | |
| | CTACAGTGCCCCTAGTCATGGTGGCACCGTCTGGGGCCCGACTA | |
| | GGTCCCTCACCCCACCTACAGGCCCTTCTCCAGGACAGACCACA | |
| | CTTCATGCATCAGCTCTCCACTGTGGATGCCCATGCCCAGACCCC | |
| | TGTGCTCCAAGTGCGTCCACTGGACAACCCAGCCATGATCAGCC | |
| | TCCCACCACCTTCTGCTGCCACTGGGGTCTTCTCCCTCAAGGCCC | |
| | GGCCTGGCCTGCCACCTGGGATCAATGTGGCCAGTCTGGAATGG | |
| | GTGTCCAGGGAGCCAGCTCTACTCTGCACCTTCCCACGCTCGGGT | |
| | ACACCCAGGAAAGACAGCAACCTTTTGGCTGCACCCCAAGGATC | |
| | CTACCCACTGCTGGCAAATGGAGTCTGCAAGTGGCCTGGTTGTG | |
| | AGAAGGTCTTCGAGGAGCCAGAAGAGTTTCTCAAGCACTGCCAA | |
| | GCAGATCATCTCCTGGATGAGAAAGGCAAGGCCCAGTGCCTCCT | |
| | CCAGAGAGAAGTGGTGCAGTCTCTGGAGCAGCAGCTGGAGCTGG | |
| | AAAAGGAGAAGCTGGGAGCTATGCAGGCCCACCTGGCTGGGAA | |
| | GATGGCGCTGGCCAAGGCTCCATCTGTGGCCTCAATGGACAAGA | |
| | GCTCTTGCTGCATCGTAGCCACCAGTACTCAGGGCAGTGTGCTCC | |
| | CGGCCTGGTCTGCTCCTCGGGAGGCTCCAGACGGCGGCCTGTTT | |
| | GCAGTGCGGAGGCACCTCTGGGGAAGCCATGGCAATAGTTCCTT | |
| | CCCAGAGTTCTTCCACAACATGGACTACTTCAAGTACCACAATAT | |
| | GCGACCCCCTTTCACCTATGCCACCCTTATCCGATGGGCCATCCT | |
| | GGAAGCCCCGGAGAGGCAGAGGACACTCAATGAAATCTACCATT | |
| | GGTTTACTCGCATGTTCGCCTACTTCAGAAACCACCCCGCCACCT | |
| | GGAAGAATGCCATCCGCCACAACCTGAGCCTGCACAAGTGCTTT | |
| | GTGCGAGTGGAGAGCGAGAAGGGAGCAGTGTGGACCGTAGATG | |
| | AATTTGAGTTTCGCAAGAAGAGGAGCCAACGCCCCAACAAGTGC | |
| | TCCAATCCCTGCCCTGAATTCTCTAGAcagtgtactaattatgctctcttgaaattgg | |
| | ctggagatgttgagagcaaccccgggccgatgcttctcctggtgacaagccttctgctctgtgagttaccaca | |
| | cccagcattcctcctgatcccacgcaaagtgtgtaacggaataggtattggtgaatttaaagactcactctcca | |
| | taaatgctacgaatattaaacacttcaaaaactgcacctccatcagtggcgatctccacatcctgccggtggc | |
| | atttaggggtgactccttcacacatactcctcctctggatccacaggaactggatattctgaaaaccgtaaagg | |
| | aaatcacagggtattgctgattcaggcttggcctgaaaacaggacggacctccatgcctttgagaacctaga | |
| | aatcatacgcggcaggaccaagcaacatggtcagttttctcttgcagtcgtcagcctgaacataacatccttg | |
| | ggattacgctccctcaaggagataagtgatggagatgtgataatttcaggaaacaaaaatttgtgctatgcaa | |
| | atacaataaactggaaaaaactgtttgggacctccggtcagaaaaccaaaattataagcaacagaggtgaa | |
| | aacagctgcaaggccacaggccaggtctgccatgccttgtgctcccccgagggctgctggggcccggag | |
| | cccagggactgcgtctcttgccggaatgtcagccgaggcagggaatgcgtggacaagtgcaaccttctgg | |
| | agggtgagccaagggagtttgtggagaactctgagtgcatacagtgccacccagagtgcctgcctcaggc | |
| | catgaacatcacctgcacaggacggggaccagacaactgtatccagtgtgcccactacattgacggcccc | |
| | cactgcgtcaagacctgcccggcaggagtcatgggagaaaacaacaccctggtctggaagtacgcagac | |
| | gccggccatgtgtgccacctgtgccatccaaactgcacctacggatgcactgggccaggtcttgaaggctg | |
| | tccaacgaatgggcctaagatcccgtccatcgccactggaTCTAGAtaaattcgagcatcttaccgcca | |
| | tttattcccatatttgttctgatttcttgatttgggtatacatttaaatgttaataaaacaaaatggtggggcaatcat | |
| | ttacatttttagggatatgtaattactagttcaggtgtattgccacaagacaaacatgttaagaaactttcccgtta | |
| | tttacgctctgttcctgttaatcaacctctggattacaaaatttgtgaaagattgactgatattcttaactatgttgct | |
| | ccttttacgctgtgtggatatgctgctttatagcctctgtatctagctattgcttcccgtacggctttcgttttctcct | |
| | ccttgtataaatcctggttgctgtctcttttagaggagttgtggcccgttgtccgtcaacgtggcgtggtgtgctc | |
| | tgtgtttgctgacgcaacccccactggctggggcattgccaccacctgtcaactcctttctgggactttcgcttt | |
| | ccccctcccgatcgccacggcagaactcatcgccgcctgccttgcccgctgctggacaggggctaggttg | |
| | ctgggcactgataattccgtggtgttgtcggggaagctgacgtcctttcctcgagggggggcccggtaccttt | |
| | aagaccaatgacttacaaggcagctgtagatcttagccactttttaaaagaaaaggggggactggaagggc | |
| | taattcactcccaacgaagacaagatctgctttttgcttgtactgggtctctctggttagaccagatctgagcct | |
| | gggagctctctggctaactagggaacccactgcttaagcctcaataaagcttgccttgagtgcttcaagtagt | |
| | gtgtgcccgtctgttgtgtgactctggtaactagagatccctcagacccttttagtcagtgtggaaaatctctag | |
| | cagtagtagttcatgtcatcttattattcagtatttataacttgcaaagaaatgaatatcagagagtgagaggaa | |
| | cttgtttattgcagcttataatggttacaaataaagcaatagcatcacaaatttcacaaataaagcattatttcact | |
| | gcattctagttgtggtttgtccaaactcatcaatgtatcttatcatgtctggctctagctatcccgcccctaactcc | |
| | gcccagttccgcccattctccgccccatggctgactaattttttttatttatgcagaggccgaggccgcctcgg | |
| | cctctgagctattccagaagtagtgaggaggcttattggaggcctaggcttttgcgtcgagacgtacccaatt | |
| | cgccctatagtgagtcgtattacgcgcgctcactggccgtcgttttacaacgtcgtgactgggaaaaccctgg | |
| | cgttacccaacttaatcgccttgcagcacatccccctttcgccagctggcgtaatagcgaagaggcccgcac | |
| | cgatcgcccttcccaacagttgcgcagcctgaatggcgaatggcgcgacgcgccctgtagcggcgcatta | |
| | agcgcggcgggtgtggtggttacgcgcagcgtgaccgctacacttgccagcgccctagcgcccgctccttt | |
| | cgctttcttcccttcctttctcgccacgttcgccggctttccccgtcaagctctaaatcgggggctccctttagg | |
| | gttccgatttagtgctttacggcacctcgaccccaaaaaacttgattagggtgatggttcacgtagtgggccat | |
| | cgccctgatagacggtttttcgccctttgacgttggagtccacgttctttaatagtggactcttgttccaaactgg | |
| | aacaacactcaaccctatctcggtctattcttttgatttataagggattttgccgatttcggcctattggttaaaaa | |
| | atgagctgatttaacaaaaatttaacgcgaattttaacaaaatattaacgtttacaatttcccaggtggcacttttc | |
| | ggggaaatgtgcgcggaacccctatttgtttatttttctaaatacattcaaatatgtatccgctcatgagacaata | |
| | accctgataaatgcttcaataatattgaaaaaggaagagtatgagtattcaacatttccgtgtcgcccttattcc | |
| | cttattgcggcattttgccttcctgatttgctcacccagaaacgctggtgaaagtaaaagatgctgaagatcag | |
| | ttgggtgcacgagtgggttacatcgaactggatctcaacagcggtaagatccttgagagttttcgccccgaa | |
| | gaacgttttccaatgatgagcacttttaaagttctgctatgtggcgcggtattatcccgtattgacgccgggcaa | |
| | gagcaactcggtcgccgcatacactattctcagaatgacttggttgagtactcaccagtcacagaaaagcat | |
| | cttacggatggcatgacagtaagagaattatgcagtgctgccataaccatgagtgataacactgcggccaac | |
| | ttacttctgacaacgatcggaggaccgaaggagctaaccgcttttttgcacaacatgggggatcatgtaactc | |
| | gccttgatcgttgggaaccggagctgaatgaagccataccaaacgacgagcgtgacaccacgatgcctgt | |
| | agcaatggcaacaacgttgcgcaaactattaactggcgaactacttactctagcttcccggcaacaattaata | |
| | gactggatggaggcggataaagttgcaggaccacttctgcgctcggcccttccggctggctggtttattgct | |
| | gataaatctggagccggtgagcgtgggtctcgcggtatcattgcagcactggggccagatggtaagccctc | |
| | ccgtatcgtagttatctacacgacggggagtcaggcaactatggatgaacgaaatagacagatcgctgaga | |
| | taggtgcctcactgattaagcattggtaactgtcagaccaagtttactcatatatactttagattgatttaaaactt | |
| | catttttaatttaaaaggatctaggtgaagatcctttttgataatctcatgaccaaaatcccttaacgtgagttttcg | |
| | ttccactgagcgtcagaccccgtagaaaagatcaaaggatcttcttgagatcattttttctgcgcgtaatctgc | |
| | tgcttgcaaacaaaaaaaccaccgctaccagcggtggtttgtttgccggatcaagagctaccaactctttttcc | |
| | gaaggtaactggcttcagcagagcgcagataccaaatactgtccttctagtgtagccgtagttaggccacca | |
| | cttcaagaactctgtagcaccgcctacatacctcgctctgctaatcctgttaccagtggctgctgccagtggc | |
| | gataagtcgtgtcttaccgggttggactcaagacgatagttaccggataaggcgcagcggtcgggctgaac | |
| | ggggggttcgtgcacacagcccagcttggagcgaacgacctacaccgaactgagatacctacagcgtga | |
| | gctatgagaaagcgccacgcttcccgaagggagaaaggcggacaggtatccggtaagcggcagggtcg | |
| | gaacaggagagcgcacgagggagcttccagggggaaacgcctggtatctttatagtcctgtcgggtttcgc | |
| | cacctctgacttgagcgtcgatttttgtgatgctcgtcaggggggcggagcctatggaaaaacgccagcaac | |
| | gcggcctttttacggttcctggccttttgctggccttttgctcacatgttctttcctgcgttatcccctgattctgtg | |
| | gataaccgtattaccgcctttgagtgagctgataccgctcgccgcagccgaacgaccgagcgcagcgagt | |
| | cagtgagcgaggaagcggaagagcgcccaatacgcaaaccgcctctccccgcgcgttggccgattcatta | |
| | atgcagctggcacgacaggtttcccgactggaaagcgggcagtgagcgcaacgcaattaatgtgagttag | |
| | ctcactcattaggcaccccaggctttacactttatgcttccggctcgtatgttgtgtggaattgtgagcggataa | |
| | caatttcacacaggaaacagctatgaccatgattacgccaagcgcgcaattaaccctcactaaagggaaca | |
| | aaagctggagctgcaagcttaatgtagtcttatgcaatactcttgtagtcttgcaacatggtaacgatgagttag | |
| | caacatgccttacaaggagagaaaaagcaccgtgcatgccgattggtggaagtaaggtggtacgatcgtg | |
| | ccttattaggaaggcaacagacgggtctgacatggattggacgaaccactgaattgccgcattgcagagat | |
| | attgtatttaagtgcctagctcgatacataaacgggtctctctggttagaccagatctgagcctgggagctctct | |
| | ggctaactagggaacccactgcttaagcctcaataaagcttgccttgagtgcttcaagtagtgtgtgcccgtct | |
| | gttgtgtgactctggtaactagagatccctcagacccttttagtcagtgtggaaaatctctagcagtggcgccc | |
| | gaacagggacttgaaagcgaaagggaaaccagaggagctctctcgacgcaggactcggcttgctgaagc | |
| | gcgcacggcaagaggcgaggggcggcgactggtgagtacgccaaaaattttgactagcggaggctaga | |
| | aggagagagatgggtgcgagagcgtcagtattaagcgggggagaattagatcgcgatgggaaaaaattc | |
| | ggttaaggccagggggaaagaaaaaatataaattaaaacatatagtatgggcaagcagggagctagaacg | |
| | attcgcagttaatcctggcctgttagaaacatcagaaggctgtagacaaatactgggacagctacaaccatc | |
| | ccttcagacaggatcagaagaacttagatcattatataatacagtagcaaccctctattgtgtgcatcaaagga | |
| | tagagataaaagacaccaaggaagctttagacaagatagaggaagagcaaaacaaaagtaagaccaccg | |
| | cacagcaagcggccgctgatcttcagacctggaggaggagatatgagggacaattggagaagtgaattat | |
| | ataaatataaagtagtaaaaattgaaccattaggagtagcacccaccaaggcaaagagaagagtggtgcag | |
| | agagaaaaaagagcagtgggaataggagctttgttccttgggttcttgggagcagcaggaagcactatggg | |
| | cgcagcctcaatgacgctgacggtacaggccagacaattattgtctggtatagtgcagcagcagaacaattt | |
| | gctgagggctattgaggcgcaacagcatctgttgcaactcacagtctggggcatcaagcagctccaggcaa | |
| | gaatcctggctgtggaaagatacctaaaggatcaacagctcctggggatttggggttgctctggaaaactca | |
| | tttgcaccactgctgtgccttggaatgctagttggagtaataaatctctggaacagatttggaatcacacgacc | |
| | tggatggagtgggacagagaaattaacaattacacaagcttaatacactccttaattgaagaatcgcaaaacc | |
| | agcaagaaaagaatgaacaagaattattggaattagataaatgggcaagtttgtggaattggtttaacataac | |
| | aaattggctgtggtatataaaattattcataatgatagtaggaggcttggtaggtttaagaatagtttttgctgtac | |
| | tttctatagtgaatagagttaggcagggatattcaccattatcgtttcagacccacctcccaaccccgagggg | |
| | acccgacaggcccgaaggaatagaagaagaaggtggagagagagacagagacagatccattcgattagt | |
| | gaacggatctcgacggtatcggtt | |
| 5 | GTCGACATTGATTATTGACTAGATCATCGCGTGAGGCTCCGGTGC | EF1 alpha |
| | CCGTCAGTGGGCAGAGCGCACATCGCCCACAGTCCCCGAGAAGT | |
| | TGGGGGGAGGGGTCGGCAATTGAACCGGTGCCTAGAGAAGGTG | |
| | GCGCGGGGTAAACTGGGAAAGTGATGTCGTGTACTGGCTCCGCC | |
| | TTTTTCCCGAGGGTGGGGGAGAACCGTATATAAGTGCAGTAGTC | |
| | GCCGTGAACGTTCTTTTTCGCAACGGGTTTGCCGCCAGAACACA | |
| | GGTAAGTGCCGTGTGTGGTTCCCGCGGGCCTGGCCTCTTTACGGG | |
| | TTATGGCCCTTGCGTGCCTTGAATTACTTCCACGCCCCTGGCTGC | |
| | AGTACGTGATTCTTGATCCCGAGCTTCGGGTTGGAAGTGGGTGG | |
| | GAGAGTTCGAGGCCTTGCGCTTAAGGAGCCCCTTCGCCTCGTGCT | |
| | TGAGTTGAGGCCTGGCCTGGGCGCTGGGGCCGCCGCGTGCGAAT | |
| | CTGGTGGCACCTTCGCGCCTGTCTCGCTGCTTTCGATAAGTCTCT | |
| | AGCCATTTAAAATTTTTGATGACCTGCTGCGACGCTTTTTTTCTG | |
| | GCAAGATAGTCTTGTAAATGCGGGCCAAGATCTGCACACTGGTA | |
| | TTTCGGTTTTTGGGGCCGCGGGCGGCGACGGGGCCCGTGCGTCC | |
| | CAGCGCACATGTTCGGCGAGGCGGGGCCTGCGAGCGCGGCCACC | |
| | GAGAATCGGACGGGGGTAGTCTCAAGCTGGCCGGCCTGCTCTGG | |
| | TGCCTGGCCTCGCGCCGCCGTGTATCGCCCCGCCCTGGGCGGCA | |
| | AGGCTGGCCCGGTCGGCACCAGTTGCGTGAGCGGAAAGATGGCC | |
| | GCTTCCCGGCCCTGCTGCAGGGAGCTCAAAATGGAGGACGCGGC | |
| | GCTCGGGAGAGCGGGCGGGTGAGTCACCCACACAAAGGAAAAG | |
| | GGCCTTTCCGTCCTCAGCCGTCGCTTCATGTGACTCCACGGAGTA | |
| | CCGGGCGCCGTCCAGGCACCTCGATTAGTTCTCGAGCTTTTGGAG | |
| | TACGTCGTCTTTAGGTTGGGGGGAGGGGTTTTATGCGATGGAGTT | |
| | TCCCCACACTGAGTGGGTGGAGACTGAAGTTAGGCCAGCTTGGC | |
| | ACTTGATGTAATTCTCCTTGGAATTTGCCCTTTTTGAGTTTGGATC | |
| | TTGGTTCATTCTCAAGCCTCAGACAGTGGTTCAAAGTTTTTTTCTT | |
| | CCATTTCAGGTGTCGTGAGGAATTCTGCAGTCGACGGTACCGCG | |
| | GGCCCGGGATCCACCGGTCGCCACCATGGTGAGCAAGGGCGAG | |
| | GAGCTGTTCACCGGGGTGGTGCCCATCCTGGTCGAGCTGGACGG | |
| | CGACGTAAACGGCCACAAGTTCAGCGTGTCCGGCGAGGGCGAG | |
| | GGCGATGCCACCTACGGCAAGCTGACCCTGAAGTTCATCTGCAC | |
| | CACCGGCAAGCTGCCCGTGCCCTGGCCCACCCTCGTGACCACCC | |
| | TGACCTACGGCGTGCAGTGCTTCAGCCGCTACCCCGACCACATG | |
| | AAGCAGCACGACTTCTTCAAGTCCGCCATGCCCGAAGGCTACGT | |
| | CCAGGAGCGCACCATCTTCTTCAAGGACGACGGCAACTACAAGA | |
| | CCCGCGCCGAGGTGAAGTTCGAGGGCGACACCCTGGTGAACCGC | |
| | ATCGAGCTGAAGGGCATCGACTTCAAGGAGGACGGCAACATCCT | |
| | GGGGCACAAGCTGGAGTACAACTACAACAGCCACAACGTCTATA | |
| | TCATGGCCGACAAGCAGAAGAACGGCATCAAGGTGAACTTCAA | |
| | GATCCGCCACAACATCGAGGACGGCAGCGTGCAGCTCGCCGACC | |
| | ACTACCAGCAGAACACCCCCATCGGCGACGGCCCCGTGCTGCTG | |
| | CCCGACAACCACTACCTGAGCACCCAGTCCGCCCTGAGCAAAGA | |
| | CCCCAACGAGAAGCGCGATCACATGGTCCTGCTGGAGTTCGTGA | |
| | CCGCCGCCGGGATCACTCTCGGCATGGACGAGCTGTACAAGTAA | |
| | AGCGGCCGCACTCCTCAGGTGCAGGCTGCCTATCAGAAGGTGGT | |
| | GGCTGGTGTGGCCAATGCCCTGGCTCACAAATACCACTGAGATC | |
| | TTTTTCCCTCTGCCAAAAATTATGGGGACATCATGAAGCCCCTTG | |
| | AGCATCTGACTTCTGGCTAATAAAGGAAATTTATTTTCATTGCAA | |
| | TAGTGTGTTGGAATTTTTTGTGTCTCTCACTCGGAAGGACATATG | |
| | GGAGGGCAAATCATTTAAAACATCAGAATGAGTATTTGGTTTAG | |
| | AGTTTGGCAACATATGCCCATATGCTGGCTGCCATGAACAAAGG | |
| | TTGGCTATAAAGAGGTCATCAGTATATGAAACAGCCCCCTGCTG | |
| | TCCATTCCTTATTCCATAGAAAAGCCTTGACTTGAGGTTAGATTT | |
| | TTTTTATATTTTGTTTTGTGTTATTTTTTTCTTTAACATCCCTAAAA | |
| | TTTTCCTTACATGTTTTACTAGCCAGATTTTTCCTCCTCTCCTGAC | |
| | TACTCCCAGTCATAGCTGTCCCTCTTCTCTTATGGAGATCCCTCG | |
| | ACCTGCAGCCCAAGCTTGGCGTAATCATGGTCATAGCTGTTTCCT | |
| | GTGTGAAATTGTTATCCGCTCACAATTCCACACAACATACGAGC | |
| | CGGAAGCATAAAGTGTAAAGCCTGGGGTGCCTAATGAGTGAGCT | |
| | AACTCACATTAATTGCGTTGCGCTCACTGCCCGCTTTCCAGTCGG | |
| | GAAACCTGTCGTGCCAGCGGATCCGCATCTCAATTAGTCAGCAA | |
| | CCATAGTCCCGCCCCTAACTCCGCCCATCCCGCCCCTAACTCCGC | |
| | CCAGTTCCGCCCATTCTCCGCCCCATGGCTGACTAATTTTTTTTAT | |
| | TTATGCAGAGGCCGAGGCCGCCTCGGCCTCTGAGCTATTCCAGA | |
| | AGTAGTGAGGAGGCTTTTTTGGAGGCCTAGGCTTTTGCAAAAAG | |
| | CTAACTTGTTTATTGCAGCTTATAATGGTTACAAATAAAGCAATA | |
| | GCATCACAAATTTCACAAATAAAGCATTTTTTTCACTGCATTCTA | |
| | GTTGTGGTTTGTCCAAACTCATCAATGTATCTTATCATGTCTGGA | |
| | TCCGCTGCATTAATGAATCGGCCAACGCGCGGGGAGAGGCGGTT | |
| | TGCGTATTGGGCGCTCTTCCGCTTCCTCGCTCACTGACTCGCTGC | |
| | GCTCGGTCGTTCGGCTGCGGCGAGCGGTATCAGCTCACTCAAAG | |
| | GCGGTAATACGGTTATCCACAGAATCAGGGGATAACGCAGGAA | |
| | AGAACATGTGAGCAAAAGGCCAGCAAAAGGCCAGGAACCGTAA | |
| | AAAGGCCGCGTTGCTGGCGTTTTTCCATAGGCTCCGCCCCCCTGA | |
| | CGAGCATCACAAAAATCGACGCTCAAGTCAGAGGTGGCGAAAC | |
| | CCGACAGGACTATAAAGATACCAGGCGTTTCCCCCTGGAAGCTC | |
| | CCTCGTGCGCTCTCCTGTTCCGACCCTGCCGCTTACCGGATACCT | |
| | GTCCGCCTTTCTCCCTTCGGGAAGCGTGGCGCTTTCTCATAGCTC | |
| | ACGCTGTAGGTATCTCAGTTCGGTGTAGGTCGTTCGCTCCAAGCT | |
| | GGGCTGTGTGCACGAACCCCCCGTTCAGCCCGACCGCTGCGCCT | |
| | TATCCGGTAACTATCGTCTTGAGTCCAACCCGGTAAGACACGAC | |
| | TTATCGCCACTGGCAGCAGCCACTGGTAACAGGATTAGCAGAGC | |
| | GAGGTATGTAGGCGGTGCTACAGAGTTCTTGAAGTGGTGGCCTA | |
| | ACTACGGCTACACTAGAAGAACAGTATTTGGTATCTGCGCTCTG | |
| | CTGAAGCCAGTTACCTTCGGAAAAAGAGTTGGTAGCTCTTGATC | |
| | CGGCAAACAAACCACCGCTGGTAGCGGTGGTTTTTTTGTTTGCAA | |
| | GCAGCAGATTACGCGCAGAAAAAAAGGATCTCAAGAAGATCCTT | |
| | TGATCTTTTCTACGGGGTCTGACGCTCAGTGGAACGAAAACTCA | |
| | CGTTAAGGGATTTTGGTCATGAGATTATCAAAAAGGATCTTCAC | |
| | CTAGATCCTTTTAAATTAAAAATGAAGTTTTAAATCAATCTAAAG | |
| | TATATATGAGTAAACTTGGTCTGACAGTTACCAATGCTTAATCAG | |
| | TGAGGCACCTATCTCAGCGATCTGTCTATTTCGTTCATCCATAGT | |
| | TGCCTGACTCCCCGTCGTGTAGATAACTACGATACGGGAGGGCT | |
| | TACCATCTGGCCCCAGTGCTGCAATGATACCGCGAGACCCACGC | |
| | TCACCGGCTCCAGATTTATCAGCAATAAACCAGCCAGCCGGAAG | |
| | GGCCGAGCGCAGAAGTGGTCCTGCAACTTTATCCGCCTCCATCC | |
| | AGTCTATTAATTGTTGCCGGGAAGCTAGAGTAAGTAGTTCGCCA | |
| | GTTAATAGTTTGCGCAACGTTGTTGCCATTGCTACAGGCATCGTG | |
| | GTGTCACGCTCGTCGTTTGGTATGGCTTCATTCAGCTCCGGTTCC | |
| | CAACGATCAAGGCGAGTTACATGATCCCCCATGTTGTGCAAAAA | |
| | AGCGGTTAGCTCCTTCGGTCCTCCGATCGTTGTCAGAAGTAAGTT | |
| | GGCCGCAGTGTTATCACTCATGGTTATGGCAGCACTGCATAATTC | |
| | TCTTACTGTCATGCCATCCGTAAGATGCTTTTCTGTGACTGGTGA | |
| | GTACTCAACCAAGTCATTCTGAGAATAGTGTATGCGGCGACCGA | |
| | GTTGCTCTTGCCCGGCGTCAATACGGGATAATACCGCGCCACAT | |
| | AGCAGAACTTTAAAAGTGCTCATCATTGGAAAACGTTCTTCGGG | |
| | GCGAAAACTCTCAAGGATCTTACCGCTGTTGAGATCCAGTTCGA | |
| | TGTAACCCACTCGTGCACCCAACTGATCTTCAGCATCTTTTACTT | |
| | TCACCAGCGTTTCTGGGTGAGCAAAAACAGGAAGGCAAAATGCC | |
| | GCAAAAAAGGGAATAAGGGCGACACGGAAATGTTGAATACTCA | |
| | TACTCTTCCTTTTTCAATATTATTGAAGCATTTATCAGGGTTATTG | |
| | TCTCATGAGCGGATACATATTTGAATGTATTTAGAAAAATAAAC | |
| | AAATAGGGGTTCCGCGCACATTTCCCCGAAAAGTGCCACCTGG | |
| 6 | GCCACGGCGGAACTCATCGCCGCCTGCCTTGCCCGCTGCTGGAC | Woodchuck |
| | AGGGGCTCGGCTGTTGGGCACTGACAATTCCGTGGT | Hepatitis |
| | | Virus Post- |
| | | tran- |
| | | scriptionl |
| | | Regulatory |
| | | Element |
| | | Alpha |
| | | Component |
| 7 | AATCAACCTCTGGATTACAAAATTTGTGAAAGATTGACTGGTATT | Woodchuck |
| | CTTAACTATGTTGCTCCTTTTACGCTATGTGGATACGCTGCTTTA | Hepatitis |
| | ATGCCTTTGTATCATGCTATTGCTTCCCGTATGGCTTTCATTTTCT | Virus |
| | CCTCCTTGTATAAATCCTGGTTGCTGTCTCTTTATGAGGAGTTGT | Post- |
| | GGCCCGTTGTCAGGCAACGTGGCGTGGTGTGCACTGTGTTTGCTG | tran- |
| | ACGCAACCCCCACTGGTTGGGGCATTGCCACCACCTGTCAGCTC | scriptional |
| | CTTTCCGGGACTTTCGCTTTCCCCCTCCCTATTGCCACGGCGGAA | Regulatory |
| | CTCATCGCCGCCTGCCTTGCCCGCTGCTGGACAGGGGCTCGGCTG | Element |
| | TTGGGCACTGACAATTCCGTGGTGTTGTCGGGGAAGCTGACGTC | |
| | CTTTCCATGGCTGCTCGCCTGTGTTGCCACCTGGATTCTGCGCGG | |
| | GACGTCCTTCTGCTACGTCCCTTCGGCCCTCAATCCAGCGGACCT | |
| | TCCTTCCCGCGGCCTGCTGCCGGCTCTGCGGCCTCTTCCGCGTCT | |
| | TCGCCTTCGCCCTCAGACGAGTCGGATCTCCCTTTGGGCCGCCTC | |
| | CCCGCCTG | |
| 8 | EGRGSLLTCGDVEENPGP | T2A |
| 9 | ATNFSLLKQAGDVEENPGP | P2A |
| 10 | QCTNYALLKLAGDVESNPGP | E2A |
| 11 | VKQTLNFDLLKLAGDVESNPGP | F2A |
| 12 | GSGEGRGSLLTCGDVEENPGP | T2A |
| 13 | GSGATNFSLLKQAGDVEENPGP | P2A |
| 14 | GSGQCTNYALLKLAGDVESNPGP | E2A |
| 15 | GSGVKQTLNFDLLKLAGDVESNPGP | F2A |
| 16 | ctgttttaaaagaaaaggggggattggggggtacagtgcaggggaaagaatagtagacataatagcaaca | 64-AAV-CAR- |
| | gacatacaaactaaagaattacaaaaacaaattacaaaaattcaaaattttatcaacaagcttgatcgatggct | T-Regs |
| | ccggtgcccgtcagtgggcagagcgcacatcgcccacagtccccgagaagttggggggaggggtcggc | sequence |
| | aattgaaccggtgcctagagaaggtggcgcggggtaaactgggaaagtgatgtcgtgtactggctccgcct | |
| | ttttcccgagggtgggggagaaccgtatataagtgcagtagtcgccgtgaacgttctttttcgcaacgggtttg | |
| | ccgccagaacacaggtgtcgtgacgcggatccacgcgtgcggccgcacgcgtaagcttagcgcagagg | |
| | cttggggcagccgagcggcagccaggccccggcccgggcctcggttccagaagggagaggagcccgc | |
| | caaggcgcgcaagagagcgggctgcctcgcagtccgagccggagagggagcgcgagccgcgccggc | |
| | cccggacggcctcgccgccaccatggctctgcccgtcaccgcactgctgctgcctctggctctgctgctgc | |
| | acgccgcaagaccagacattgtgctgacacagtctcctgcttccttagctgtatctctggggcagagggcca | |
| | ccatctcatgcagggccagccaaagtgtcagttcatctacctataattttatacactggtatcaacagaaatca | |
| | ggacagccacccaaactcctcatcaagtatgcatccaacctagaatctggggtccctgtcaggttcagtggc | |
| | agtgggtctgggacagacttctccctcaacatccatcctgtggaggaggaggatactgcaacatattactgtc | |
| | agcacagttgggagattccatacacgttcggagggggggccaagttggagataaaaggcagtactagcgg | |
| | tggtggctccgggggcggttccggtgggggcggcagcagccaggtgcagctgaaggagtcaggacctg | |
| | gcctggtggcaccctcacagagcctgtccatcacatgcaccgtctcaggattctcattaagcggctatggcat | |
| | aagctgggttcgccagcctccaggaaagcgtctggaatggctgggattgatatggggtgatggaaccaca | |
| | gactataattcagctctcaaatccagactgagcatcagcaaggacaactccaagagccaggttttcttaaaaa | |
| | tgaacagtctgcaaactgatgacacagccaggtacttctgtaccagagggcctccggccttctataagtacct | |
| | ctactttgactactggggccaaggcaccactctcacagtctcctcactcgaccccaaatcttctgacaaaact | |
| | cacacatgcccaccgtgcccagcacctcctgtggcaggaccgtcagtcttcctcttccccccaaaacccaa | |
| | ggacaccctcatgatctcccggacccctgaggtcacatgcgtggtggtggacgtgagccacgaagaccct | |
| | gaggtcaagttcaactggtacgtggacggcgtggaggtgcataatgccaagacaaagccgcgggaggag | |
| | cagtaccagagcacgtaccgtgtggtcagcgtcctcaccgtcctgcaccaggactggctgaatggcaagg | |
| | agtacaagtgcaaggtctccaacaaagccctcccagcccccatcgagaaaaccatctccaaagccaaagg | |
| | gcagccccgagaaccacaggtgtacaccctgccaccatcacgagatgagctgaccaagaaccaggtcag | |
| | cctgacctgcctggtcaaaggcttctatcccagcgacatcgccgtggagtgggagagcaatgggcagccg | |
| | gagaacaactacaagaccacgcctcccgtgctggactccgacggctccttcttcctctacagcaagctcacc | |
| | gtggacaagagcaggtggcagcaggggaacgtcttctcatgctccgtgatgcatgaggctctgcacaacc | |
| | actacacgcagaagagcctctccctgtctcccttttgggtgctggtggtggttggtggagtcctggcttgctat | |
| | agcttgctagtaacagtggcctttattattttctgggtgaggagtaagaggagcaggggaggtcacagtgact | |
| | acatgaacatgactccccgccgccccgggcccacccgcaagcattaccagccctatgccccaccacgcg | |
| | acttcgcagcctatcgctccaaacggggcagaaagaaactcctgtatatattcaaacaaccatttatgagacc | |
| | agtacaaactactcaagaggaagatggctgtagctgccgatttccagaagaagaagaaggaggatgtgaa | |
| | ctgagagtgaagttcagcaggagcgcagacgcccccgcgtaccagcagggccagaaccagctctataac | |
| | gagctcaatctaggacgaagagaggagtacgatgttttggacaagagacgtggccgggaccctgagatgg | |
| | ggggaaagccgagaaggaagaaccctcaggaaggcctgtacaatgaactgcagaaagataagatggcg | |
| | gaggcctacagtgagattgggatgaaaggcgagcgccggaggggcaaggggcacgatggcctttacca | |
| | gggtctcagtacagccaccaaggacacctacgacgcccttcacatgcaggccctgccccctcgccagtgt | |
| | actaattatgctctcttgaaattggctggagatgttgagagcaaccccgggccggaattcatgcccaacccta | |
| | ggccagccaagcctatggctccttccttggcccttggcccatccccaggagtcttgccaagctggaagactg | |
| | cacccaagggctcagaacttctagggaccaggggctctgggggacccttccaaggtcgggacctgcgaa | |
| | gtggggcccacacctcttcttccttgaaccccctgccaccatcccagctgcagctgcctacagtgcccctagt | |
| | catggtggcaccgtctggggcccgactaggtccctcaccccacctacaggcccttctccaggacagacca | |
| | cacttcatgcatcagctctccactgtggatgcccatgcccagacccctgtgctccaagtgcgtccactggac | |
| | aacccagccatgatcagcctcccaccaccttctgctgccactggggtcttctccctcaaggcccggcctggc | |
| | ctgccacctgggatcaatgtggccagtctggaatgggtgtccagggagccagctctactctgcaccttccca | |
| | cgctcgggtacacccaggaaagacagcaaccttttggctgcaccccaaggatcctacccactgctggcaa | |
| | atggagtctgcaagtggcctggttgtgagaaggtcttcgaggagccagaagagtttctcaagcactgccaa | |
| | gcagatcatctcctggatgagaaaggcaaggcccagtgcctcctccagagagaagtggtgcagtctctgg | |
| | agcagcagctggagctggaaaaggagaagctgggagctatgcaggcccacctggctgggaagatggcg | |
| | ctggccaaggctccatctgtggcctcaatggacaagagctcttgctgcatcgtagccaccagtactcagggc | |
| | agtgtgctcccggcctggtctgctcctcgggaggctccagacggcggcctgtttgcagtgcggaggcacct | |
| | ctggggaagccatggcaatagttccttcccagagttcttccacaacatggactacttcaagtaccacaatatg | |
| | cgaccccctttcacctatgccacccttatccgatgggccatcctggaagccccggagaggcagaggacact | |
| | caatgaaatctaccattggtttactcgcatgttcgcctacttcagaaaccaccccgccacctggaagaatgcc | |
| | atccgccacaacctgagcctgcacaagtgctttgtgcgagtggagagcgagaagggagcagtgtggacc | |
| | gtagatgaatttgagtttcgcaagaagaggagccaacgccccaacaagtgctccaatccctgcccccagtg | |
| | tactaattatgctctcttgaaattggctggagatgttgagagcaaccccgggccgatgccacctcctcgcctc | |
| | ctcttcttcctcctcttcctcacccccatggaagtcaggcccgaggaacctctagtggtgaaggtggaagag | |
| | ggagataacgctgtgctgcagtgcctcaaggggacctcagatggccccactcagcagctgacctggtctc | |
| | gggagtccccgcttaaacccttcttaaaactcagcctggggctgccaggcctgggaatccacatgaggccc | |
| | ctggccatctggcttttcatcttcaacgtctctcaacagatggggggcttctacctgtgccagccggggcccc | |
| | cctctgagaaggcctggcagcctggctggacagtcaatgtggagggcagcggggagctgttccggtgga | |
| | atgtttcggacctaggtggcctgggctgtggcctgaagaacaggtcctcagagggccccagctccccttcc | |
| | gggaagctcatgagccccaagctgtatgtgtgggccaaagaccgccctgagatctgggagggagagcct | |
| | ccgtgtgtcccaccgagggacagcctgaaccagagcctcagccaggacctcaccatggcccctggctcc | |
| | acactctggctgtcctgtggggtaccccctgactctgtgtccaggggccccctctcctggacccatgtgcac | |
| | cccaaggggcctaagtcattgctgagcctagagctgaaggacgatcgcccggccagagatatgtgggtaa | |
| | tggagacgggtctgttgttgccccgggccacagctcaagacgctggaaagtattattgtcaccgtggcaacc | |
| | tgaccatgtcattccacctggagatcactgctcggccagtactatggcactggctgctgaggactggtggct | |
| | ggaaggtctcagctgtgactttggcttatctgatcttctgcctgtgttcccttgtgggcattcttcatcttcaaaga | |
| | gccctggtcctgaggaggaaaagatgatctagataaattcgagcatcttaccgccatttattcccatatttgttc | |
| | tgtttttcttgatttgggtatacatttaaatgttaataaaacaaaatggtggggcaatcatttacatttttagggatat | |
| | gtaattactagttcaggtgtattgccacaagacaaacatgttaagaaactttcccgttatttacgctctgttcctgt | |
| | taatcaacctctggattacaaaatttgtgaaagattgactgatattcttaactatgttgctccttttacgctgtgtgg | |
| | atatgctgctttatagcctctgtatctagctattgcttcccgtacggctttcgttttctcctccttgtataaatcctgg | |
| | ttgctgtctcttttagaggagttgtggcccgttgtccgtcaacgtggcgtggtgtgctctgtgtttgctgacgca | |
| | acccccactggctggggcattgccaccacctgtcaactcctttctgggactttcgctttccccctcccgatcgc | |
| | cacggcagaactcatcgccgcctgccttgcccgctgctggacaggggctaggttgctgggcactgataatt | |
| | ccgtggtgttgtcggggaagctgacgtcctttcctcgagggggggcccggtacctttaagaccaatgactta | |
| | caaggcagctgtagatcttagccactttttaaaagaaaaggggggactggaagggctaattcactcccaacg | |
| | aagacaagatctgctttttgcttgtactgggtctctctggttagaccagatctgagcctgggagctctctggcta | |
| | actagggaacccactgcttaagcctcaataaagcttgccttgagtgcttcaagtagtgtgtgcccgtctgttgt | |
| | gtgactctggtaactagagatccctcagacccttttagtcagtgtggaaaatctctagcagtagtagttcatgtc | |
| | atcttattattcagtatttataacttgcaaagaaatgaatatcagagagtgagaggaacttgtttattgcagcttat | |
| | aatggttacaaataaagcaatagcatcacaaatttcacaaataaagcatttttttcactgcattctagttgtggttt | |
| | gtccaaactcatcaatgtatcttatcatgtctggctctagctatcccgcccctaactccgcccagttccgcccat | |
| | tctccgccccatggctgactaattattttatttatgcagaggccgaggccgcctcggcctctgagctattccag | |
| | aagtagtgaggaggcttttttggaggcctaggcttttgcgtcgagacgtacccaattcgccctatagtgagtc | |
| | gtattacgcgcgctcactggccgtcgttttacaacgtcgtgactgggaaaaccctggcgttacccaacttaat | |
| | cgccttgcagcacatccccctttcgccagctggcgtaatagcgaagaggcccgcaccgatcgcccttccca | |
| | acagttgcgcagcctgaatggcgaatggcgcgacgcgccctgtagcggcgcattaagcgcggcgggtgt | |
| | ggtggttacgcgcagcgtgaccgctacacttgccagcgccctagcgcccgctcctttcgctttcttcccttcct | |
| | ttctcgccacgttcgccggctttccccgtcaagctctaaatcgggggctccctttagggttccgatttagtgctt | |
| | tacggcacctcgaccccaaaaaacttgattagggtgatggttcacgtagtgggccatcgccctgatagacg | |
| | gtttttcgccctttgacgttggagtccacgttctttaatagtggactcttgttccaaactggaacaacactcaacc | |
| | ctatctcggtctattcttttgatttataagggattttgccgatttcggcctattggttaaaaaatgagctgatttaac | |
| | aaaaatttaacgcgaattttaacaaaatattaacgtttacaatttcccaggtggcacttttcggggaaatgtgcg | |
| | cggaacccctatttgtttatttttctaaatacattcaaatatgtatccgctcatgagacaataaccctgataaatgc | |
| | ttcaataatattgaaaaaggaagagtatgagtattcaacatttccgtgtcgcccttattcccttattgcggcatttt | |
| | gccttcctgtttttgctcacccagaaacgctggtgaaagtaaaagatgctgaagatcagttgggtgcacgagt | |
| | gggttacatcgaactggatctcaacagcggtaagatccttgagagttttcgccccgaagaacgttttccaatg | |
| | atgagcacttttaaagttctgctatgtggcgcggtattatcccgtattgacgccgggcaagagcaactcggtc | |
| | gccgcatacactattctcagaatgacttggttgagtactcaccagtcacagaaaagcatcttacggatggcat | |
| | gacagtaagagaattatgcagtgctgccataaccatgagtgataacactgcggccaacttacttctgacaac | |
| | gatcggaggaccgaaggagctaaccgcttttttgcacaacatgggggatcatgtaactcgccttgatcgttg | |
| | ggaaccggagctgaatgaagccataccaaacgacgagcgtgacaccacgatgcctgtagcaatggcaac | |
| | aacgttgcgcaaactattaactggcgaactacttactctagcttcccggcaacaattaatagactggatggag | |
| | gcggataaagttgcaggaccacttctgcgctcggcccttccggctggctggtttattgctgataaatctggag | |
| | ccggtgagcgtgggtctcgcggtatcattgcagcactggggccagatggtaagccctcccgtatcgtagtta | |
| | tctacacgacggggagtcaggcaactatggatgaacgaaatagacagatcgctgagataggtgcctcactg | |
| | attaagcattggtaactgtcagaccaagtttactcatatatactttagattgatttaaaacttcatttttaatttaaaa | |
| | ggatctaggtgaagatcctttttgataatctcatgaccaaaatcccttaacgtgagttttcgttccactgagcgtc | |
| | agaccccgtagaaaagatcaaaggatcttcttgagatcctttttttctgcgcgtaatctgctgcttgcaaacaaa | |
| | aaaaccaccgctaccagcggtggtttgtttgccggatcaagagctaccaactctttttccgaaggtaactggc | |
| | ttcagcagagcgcagataccaaatactgtccttctagtgtagccgtagttaggccaccacttcaagaactctgt | |
| | agcaccgcctacatacctcgctctgctaatcctgttaccagtggctgctgccagtggcgataagtcgtgtctta | |
| | ccgggttggactcaagacgatagttaccggataaggcgcagcggtcgggctgaacggggggttcgtgca | |
| | cacagcccagcttggagcgaacgacctacaccgaactgagatacctacagcgtgagctatgagaaagcg | |
| | ccacgcttcccgaagggagaaaggcggacaggtatccggtaagcggcagggtcggaacaggagagcg | |
| | cacgagggagcttccagggggaaacgcctggtatctttatagtcctgtcgggtttcgccacctctgacttgag | |
| | cgtcgatttttgtgatgctcgtcaggggggcggagcctatggaaaaacgccagcaacgcggcctttttacgg | |
| | ttcctggccttttgctggccttttgctcacatgttctttcctgcgttatcccctgattctgtggataaccgtattacc | |
| | gcctttgagtgagctgataccgctcgccgcagccgaacgaccgagcgcagcgagtcagtgagcgaggaa | |
| | gcggaagagcgcccaatacgcaaaccgcctctccccgcgcgttggccgattcattaatgcagctggcacg | |
| | acaggtttcccgactggaaagcgggcagtgagcgcaacgcaattaatgtgagttagctcactcattaggca | |
| | ccccaggctttacactttatgcttccggctcgtatgttgtgtggaattgtgagcggataacaatttcacacagga | |
| | aacagctatgaccatgattacgccaagcgcgcaattaaccctcactaaagggaacaaaagctggagctgca | |
| | agcttaatgtagtcttatgcaatactcttgtagtcttgcaacatggtaacgatgagttagcaacatgccttacaa | |
| | ggagagaaaaagcaccgtgcatgccgattggtggaagtaaggtggtacgatcgtgccttattaggaaggc | |
| | aacagacgggtctgacatggattggacgaaccactgaattgccgcattgcagagatattgtatttaagtgcct | |
| | agctcgatacataaacgggtctctctggttagaccagatctgagcctgggagctctctggctaactagggaa | |
| | cccactgcttaagcctcaataaagcttgccttgagtgcttcaagtagtgtgtgcccgtctgttgtgtgactctgg | |
| | taactagagatccctcagacccttttagtcagtgtggaaaatctctagcagtggcgcccgaacagggacttg | |
| | aaagcgaaagggaaaccagaggagctctctcgacgcaggactcggcttgctgaagcgcgcacggcaag | |
| | aggcgaggggcggcgactggtgagtacgccaaaaattttgactagcggaggctagaaggagagagatg | |
| | ggtgcgagagcgtcagtattaagcgggggagaattagatcgcgatgggaaaaaattcggttaaggccagg | |
| | gggaaagaaaaaatataaattaaaacatatagtatgggcaagcagggagctagaacgattcgcagttaatc | |
| | ctggcctgttagaaacatcagaaggctgtagacaaatactgggacagctacaaccatcccttcagacaggat | |
| | cagaagaacttagatcattatataatacagtagcaaccctctattgtgtgcatcaaaggatagagataaaaga | |
| | caccaaggaagctttagacaagatagaggaagagcaaaacaaaagtaagaccaccgcacagcaagcgg | |
| | ccgctgatcttcagacctggaggaggagatatgagggacaattggagaagtgaattatataaatataaagta | |
| | gtaaaaattgaaccattaggagtagcacccaccaaggcaaagagaagagtggtgcagagagaaaaaaga | |
| | gcagtgggaataggagctttgttccttgggttcttgggagcagcaggaagcactatgggcgcagcctcaat | |
| | gacgctgacggtacaggccagacaattattgtctggtatagtgcagcagcagaacaatttgctgagggctat | |
| | tgaggcgcaacagcatctgttgcaactcacagtctggggcatcaagcagctccaggcaagaatcctggctg | |
| | tggaaagatacctaaaggatcaacagctcctggggatttggggttgctctggaaaactcatttgcaccactgc | |
| | tgtgccttggaatgctagttggagtaataaatctctggaacagatttggaatcacacgacctggatggagtgg | |
| | gacagagaaattaacaattacacaagcttaatacactccttaattgaagaatcgcaaaaccagcaagaaaag | |
| | aatgaacaagaattattggaattagataaatgggcaagtttgtggaattggtttaacataacaaattggctgtgg | |
| | tatataaaattattcataatgatagtaggaggcttggtaggtttaagaatagtttttgctgtactttctatagtgaat | |
| | agagttaggcagggatattcaccattatcgtttcagacccacctcccaaccccgaggggacccgacaggcc | |
| | cgaaggaatagaagaagaaggtggagagagagacagagacagatccattcgattagtgaacggatctcg | |
| | acggtatcggtt | |
| 17 | ctgttttaaaagaaaaggggggattggggggtacagtgcaggggaaagaatagtagacataatagcaaca | 65-AAV-CAR- |
| | gacatacaaactaaagaattacaaaaacaaattacaaaaattcaaaattttatcaacaagcttgatcgatggct | T-Cells |
| | ccggtgcccgtcagtgggcagagcgcacatcgcccacagtccccgagaagttggggggaggggtcggc | sequence |
| | aattgaaccggtgcctagagaaggtggcgcggggtaaactgggaaagtgatgtcgtgtactggctccgcct | |
| | ttttcccgagggtgggggagaaccgtatataagtgcagtagtcgccgtgaacgttctttttcgcaacgggtttg | |
| | ccgccagaacacaggtgtcgtgacgcggatccacgcgtgcggccgcacgcgtaagcttagcgcagagg | |
| | cttggggcagccgagcggcagccaggccccggcccgggcctcggttccagaagggagaggagcccgc | |
| | caaggcgcgcaagagagcgggctgcctcgcagtccgagccggagagggagcgcgagccgcgccggc | |
| | cccggacggcctcgccgccaccatggctctgcccgtcaccgcactgctgctgcctctggctctgctgctgc | |
| | acgccgcaagaccagacattgtgctgacacagtctcctgcttccttagctgtatctctggggcagagggcca | |
| | ccatctcatgcagggccagccaaagtgtcagttcatctacctataattttatacactggtatcaacagaaatca | |
| | ggacagccacccaaactcctcatcaagtatgcatccaacctagaatctggggtccctgtcaggttcagtggc | |
| | agtgggtctgggacagacttctccctcaacatccatcctgtggaggaggaggatactgcaacatattactgtc | |
| | agcacagttgggagattccatacacgttcggagggggggccaagttggagataaaaggcagtactagcgg | |
| | tggtggctccgggggcggttccggtgggggcggcagcagccaggtgcagctgaaggagtcaggacctg | |
| | gcctggtggcaccctcacagagcctgtccatcacatgcaccgtctcaggattctcattaagcggctatggcat | |
| | aagctgggttcgccagcctccaggaaagcgtctggaatggctgggattgatatggggtgatggaaccaca | |
| | gactataattcagctctcaaatccagactgagcatcagcaaggacaactccaagagccaggttttcttaaaaa | |
| | tgaacagtctgcaaactgatgacacagccaggtacttctgtaccagagggcctccggccttctataagtacct | |
| | ctactttgactactggggccaaggcaccactctcacagtctcctcactcgaccccaaatcttctgacaaaact | |
| | cacacatgcccaccgtgcccagcacctcctgtggcaggaccgtcagtcttcctcttccccccaaaacccaa | |
| | ggacaccctcatgatctcccggacccctgaggtcacatgcgtggtggtggacgtgagccacgaagaccct | |
| | gaggtcaagttcaactggtacgtggacggcgtggaggtgcataatgccaagacaaagccgcgggaggag | |
| | cagtaccagagcacgtaccgtgtggtcagcgtcctcaccgtcctgcaccaggactggctgaatggcaagg | |
| | agtacaagtgcaaggtctccaacaaagccctcccagcccccatcgagaaaaccatctccaaagccaaagg | |
| | gcagccccgagaaccacaggtgtacaccctgccaccatcacgagatgagctgaccaagaaccaggtcag | |
| | cctgacctgcctggtcaaaggcttctatcccagcgacatcgccgtggagtgggagagcaatgggcagccg | |
| | gagaacaactacaagaccacgcctcccgtgctggactccgacggctccttcttcctctacagcaagctcacc | |
| | gtggacaagagcaggtggcagcaggggaacgtcttctcatgctccgtgatgcatgaggctctgcacaacc | |
| | actacacgcagaagagcctctccctgtctcccttttgggtgctggtggtggttggtggagtcctggcttgctat | |
| | agcttgctagtaacagtggcctttattattttctgggtgaggagtaagaggagcaggggaggtcacagtgact | |
| | acatgaacatgactccccgccgccccgggcccacccgcaagcattaccagccctatgccccaccacgcg | |
| | acttcgcagcctatcgctccaaacggggcagaaagaaactcctgtatatattcaaacaaccatttatgagacc | |
| | agtacaaactactcaagaggaagatggctgtagctgccgatttccagaagaagaagaaggaggatgtgaa | |
| | ctgagagtgaagttcagcaggagcgcagacgcccccgcgtaccagcagggccagaaccagctctataac | |
| | gagctcaatctaggacgaagagaggagtacgatgttttggacaagagacgtggccgggaccctgagatgg | |
| | ggggaaagccgagaaggaagaaccctcaggaaggcctgtacaatgaactgcagaaagataagatggcg | |
| | gaggcctacagtgagattgggatgaaaggcgagcgccggaggggcaaggggcacgatggcctttacca | |
| | gggtctcagtacagccaccaaggacacctacgacgcccttcacatgcaggccctgccccctcgccagtgt | |
| | actaattatgctctcttgaaattggctggagatgttgagagcaaccccgggccggaattcatgccacctcctc | |
| | gcctcctcttcttcctcctcttcctcacccccatggaagtcaggcccgaggaacctctagtggtgaaggtgga | |
| | agagggagataacgctgtgctgcagtgcctcaaggggacctcagatggccccactcagcagctgacctgg | |
| | tctcgggagtccccgcttaaacccttcttaaaactcagcctggggctgccaggcctgggaatccacatgagg | |
| | cccctggccatctggcttttcatcttcaacgtctctcaacagatggggggcttctacctgtgccagccggggc | |
| | ccccctctgagaaggcctggcagcctggctggacagtcaatgtggagggcagcggggagctgttccggt | |
| | ggaatgtttcggacctaggtggcctgggctgtggcctgaagaacaggtcctcagagggccccagctcccct | |
| | tccgggaagctcatgagccccaagctgtatgtgtgggccaaagaccgccctgagatctgggagggagag | |
| | cctccgtgtgtcccaccgagggacagcctgaaccagagcctcagccaggacctcaccatggcccctggct | |
| | ccacactctggctgtcctgtggggtaccccctgactctgtgtccaggggccccctctcctggacccatgtgc | |
| | accccaaggggcctaagtcattgctgagcctagagctgaaggacgatcgcccggccagagatatgtgggt | |
| | aatggagacgggtctgttgttgccccgggccacagctcaagacgctggaaagtattattgtcaccgtggcaa | |
| | cctgaccatgtcattccacctggagatcactgctcggccagtactatggcactggctgctgaggactggtgg | |
| | ctggaaggtctcagctgtgactttggcttatctgatcttctgcctgtgttcccttgtgggcattcttcatcttcaaa | |
| | gagccctggtcctgaggaggaaaagatgatctagataaattcgagcatcttaccgccatttattcccatatttgt | |
| | tctgtttttcttgatttgggtatacatttaaatgttaataaaacaaaatggtggggcaatcatttacatttttagggat | |
| | atgtaattactagttcaggtgtattgccacaagacaaacatgttaagaaactttcccgttatttacgctctgttcct | |
| | gttaatcaacctctggattacaaaatttgtgaaagattgactgatattcttaactatgttgctccttttacgctgtgt | |
| | ggatatgctgctttatagcctctgtatctagctattgcttcccgtacggctttcgttttctcctccttgtataaatcct | |
| | ggttgctgtctcttttagaggagttgtggcccgttgtccgtcaacgtggcgtggtgtgctctgtgtttgctgacg | |
| | caacccccactggctggggcattgccaccacctgtcaactcctttctgggactttcgctttccccctcccgatc | |
| | gccacggcagaactcatcgccgcctgccttgcccgctgctggacaggggctaggttgctgggcactgata | |
| | attccgtggtgttgtcggggaagctgacgtcctttcctcgagggggggcccggtacctttaagaccaatgac | |
| | ttacaaggcagctgtagatcttagccactttttaaaagaaaaggggggactggaagggctaattcactccca | |
| | acgaagacaagatctgctttttgcttgtactgggtctctctggttagaccagatctgagcctgggagctctctg | |
| | gctaactagggaacccactgcttaagcctcaataaagcttgccttgagtgcttcaagtagtgtgtgcccgtctg | |
| | ttgtgtgactctggtaactagagatccctcagacccttttagtcagtgtggaaaatctctagcagtagtagttcat | |
| | gtcatcttattattcagtatttataacttgcaaagaaatgaatatcagagagtgagaggaacttgtttattgcagct | |
| | tataatggttacaaataaagcaatagcatcacaaatttcacaaataaagcatttttttcactgcattctagttgtgg | |
| | tttgtccaaactcatcaatgtatcttatcatgtctggctctagctatcccgcccctaactccgcccagttccgccc | |
| | attctccgccccatggctgactaattattttatttatgcagaggccgaggccgcctcggcctctgagctattcc | |
| | agaagtagtgaggaggcttttttggaggcctaggcttttgcgtcgagacgtacccaattcgccctatagtgag | |
| | tcgtattacgcgcgctcactggccgtcgttttacaacgtcgtgactgggaaaaccctggcgttacccaactta | |
| | atcgccttgcagcacatccccctttcgccagctggcgtaatagcgaagaggcccgcaccgatcgcccttcc | |
| | caacagttgcgcagcctgaatggcgaatggcgcgacgcgccctgtagcggcgcattaagcgcggcgggt | |
| | gtggtggttacgcgcagcgtgaccgctacacttgccagcgccctagcgcccgctcctttcgctttcttcccttc | |
| | ctttctcgccacgttcgccggctttccccgtcaagctctaaatcgggggctccctttagggttccgatttagtgc | |
| | tttacggcacctcgaccccaaaaaacttgattagggtgatggttcacgtagtgggccatcgccctgatagac | |
| | ggtttttcgccctttgacgttggagtccacgttctttaatagtggactcttgttccaaactggaacaacactcaac | |
| | cctatctcggtctattcttttgatttataagggattttgccgatttcggcctattggttaaaaaatgagctgatttaa | |
| | caaaaatttaacgcgaattttaacaaaatattaacgtttacaatttcccaggtggcacttttcggggaaatgtgc | |
| | gcggaacccctatttgtttatttttctaaatacattcaaatatgtatccgctcatgagacaataaccctgataaatg | |
| | cttcaataatattgaaaaaggaagagtatgagtattcaacatttccgtgtcgcccttattcccttattgcggcattt | |
| | tgccttcctgtttttgctcacccagaaacgctggtgaaagtaaaagatgctgaagatcagttgggtgcacgag | |
| | tgggttacatcgaactggatctcaacagcggtaagatccttgagagttttcgccccgaagaacgttttccaatg | |
| | atgagcacttttaaagttctgctatgtggcgcggtattatcccgtattgacgccgggcaagagcaactcggtc | |
| | gccgcatacactattctcagaatgacttggttgagtactcaccagtcacagaaaagcatcttacggatggcat | |
| | gacagtaagagaattatgcagtgctgccataaccatgagtgataacactgcggccaacttacttctgacaac | |
| | gatcggaggaccgaaggagctaaccgcttttttgcacaacatgggggatcatgtaactcgccttgatcgttg | |
| | ggaaccggagctgaatgaagccataccaaacgacgagcgtgacaccacgatgcctgtagcaatggcaac | |
| | aacgttgcgcaaactattaactggcgaactacttactctagcttcccggcaacaattaatagactggatggag | |
| | gcggataaagttgcaggaccacttctgcgctcggcccttccggctggctggtttattgctgataaatctggag | |
| | ccggtgagcgtgggtctcgcggtatcattgcagcactggggccagatggtaagccctcccgtatcgtagtta | |
| | tctacacgacggggagtcaggcaactatggatgaacgaaatagacagatcgctgagataggtgcctcactg | |
| | attaagcattggtaactgtcagaccaagtttactcatatatactttagattgatttaaaacttcatttttaatttaaaa | |
| | ggatctaggtgaagatcctttttgataatctcatgaccaaaatcccttaacgtgagttttcgttccactgagcgtc | |
| | agaccccgtagaaaagatcaaaggatcttcttgagatcctttttttctgcgcgtaatctgctgcttgcaaacaaa | |
| | aaaaccaccgctaccagcggtggtttgtttgccggatcaagagctaccaactctttttccgaaggtaactggc | |
| | ttcagcagagcgcagataccaaatactgtccttctagtgtagccgtagttaggccaccacttcaagaactctgt | |
| | agcaccgcctacatacctcgctctgctaatcctgttaccagtggctgctgccagtggcgataagtcgtgtctta | |
| | ccgggttggactcaagacgatagttaccggataaggcgcagcggtcgggctgaacggggggttcgtgca | |
| | cacagcccagcttggagcgaacgacctacaccgaactgagatacctacagcgtgagctatgagaaagcg | |
| | ccacgcttcccgaagggagaaaggcggacaggtatccggtaagcggcagggtcggaacaggagagcg | |
| | cacgagggagcttccagggggaaacgcctggtatctttatagtcctgtcgggtttcgccacctctgacttgag | |
| | cgtcgatttttgtgatgctcgtcaggggggcggagcctatggaaaaacgccagcaacgcggcctttttacgg | |
| | ttcctggccttttgctggccttttgctcacatgttctttcctgcgttatcccctgattctgtggataaccgtattacc | |
| | gcctttgagtgagctgataccgctcgccgcagccgaacgaccgagcgcagcgagtcagtgagcgaggaa | |
| | gcggaagagcgcccaatacgcaaaccgcctctccccgcgcgttggccgattcattaatgcagctggcacg | |
| | acaggtttcccgactggaaagcgggcagtgagcgcaacgcaattaatgtgagttagctcactcattaggca | |
| | ccccaggctttacactttatgcttccggctcgtatgttgtgtggaattgtgagcggataacaatttcacacagga | |
| | aacagctatgaccatgattacgccaagcgcgcaattaaccctcactaaagggaacaaaagctggagctgca | |
| | agcttaatgtagtcttatgcaatactcttgtagtcttgcaacatggtaacgatgagttagcaacatgccttacaa | |
| | ggagagaaaaagcaccgtgcatgccgattggtggaagtaaggtggtacgatcgtgccttattaggaaggc | |
| | aacagacgggtctgacatggattggacgaaccactgaattgccgcattgcagagatattgtatttaagtgcct | |
| | agctcgatacataaacgggtctctctggttagaccagatctgagcctgggagctctctggctaactagggaa | |
| | cccactgcttaagcctcaataaagcttgccttgagtgcttcaagtagtgtgtgcccgtctgttgtgtgactctgg | |
| | taactagagatccctcagacccttttagtcagtgtggaaaatctctagcagtggcgcccgaacagggacttg | |
| | aaagcgaaagggaaaccagaggagctctctcgacgcaggactcggcttgctgaagcgcgcacggcaag | |
| | aggcgaggggcggcgactggtgagtacgccaaaaattttgactagcggaggctagaaggagagagatg | |
| | ggtgcgagagcgtcagtattaagcgggggagaattagatcgcgatgggaaaaaattcggttaaggccagg | |
| | gggaaagaaaaaatataaattaaaacatatagtatgggcaagcagggagctagaacgattcgcagttaatc | |
| | ctggcctgttagaaacatcagaaggctgtagacaaatactgggacagctacaaccatcccttcagacaggat | |
| | cagaagaacttagatcattatataatacagtagcaaccctctattgtgtgcatcaaaggatagagataaaaga | |
| | caccaaggaagctttagacaagatagaggaagagcaaaacaaaagtaagaccaccgcacagcaagcgg | |
| | ccgctgatcttcagacctggaggaggagatatgagggacaattggagaagtgaattatataaatataaagta | |
| | gtaaaaattgaaccattaggagtagcacccaccaaggcaaagagaagagtggtgcagagagaaaaaaga | |
| | gcagtgggaataggagctttgttccttgggttcttgggagcagcaggaagcactatgggcgcagcctcaat | |
| | gacgctgacggtacaggccagacaattattgtctggtatagtgcagcagcagaacaatttgctgagggctat | |
| | tgaggcgcaacagcatctgttgcaactcacagtctggggcatcaagcagctccaggcaagaatcctggctg | |
| | tggaaagatacctaaaggatcaacagctcctggggatttggggttgctctggaaaactcatttgcaccactgc | |
| | tgtgccttggaatgctagttggagtaataaatctctggaacagatttggaatcacacgacctggatggagtgg | |
| | gacagagaaattaacaattacacaagcttaatacactccttaattgaagaatcgcaaaaccagcaagaaaag | |
| | aatgaacaagaattattggaattagataaatgggcaagtttgtggaattggtttaacataacaaattggctgtgg | |
| | tatataaaattattcataatgatagtaggaggcttggtaggtttaagaatagtttttgctgtactttctatagtgaat | |
| | agagttaggcagggatattcaccattatcgtttcagacccacctcccaaccccgaggggacccgacaggcc | |
| | cgaaggaatagaagaagaaggtggagagagagacagagacagatccattcgattagtgaacggatctcg | |
| | acggtatcggtt | |
| 18 | aacaagcttgatcgatggctccggtgcccgtcagtgggcagagcgcacatcgcccacagtccccgagaa | EF1-alpha |
| | gttggggggaggggtcggcaattgaaccggtgcctagagaaggtggcgcggggtaaactgggaaagtg | |
| | atgtcgtgtactggctccgcctttttcccgagggtgggggagaaccgtatataagtgcagtagtcgccgtga | |
| | acgttctttttcgcaacgggtttgccgccagaacacaggtgtcgtgacgcggatccacgcgtgcggccgca | |
| | cgcgtaagctt | |
| 19 | GGCATTGATTATTGACTAGTTATTAATAGTAATCAATTACGGGGT | CMV |
| | CATTAGTTCATAGCCCATATATGGAGTTCCGCGTTACATAACTTA | |
| | CGGTAAATGGCCCGCCTGGCTGACCGCCCAACGACCCCCGCCCA | |
| | TTGACGTCAATAATGACGTATGTTCCCATAGTAACGCCAATAGG | |
| | GACTTTCCATTGACGTCAATGGGTGGAGTATTTACGGTAAACTGC | |
| | CCACTTGGCAGTACATCAAGTGTATCATATGCCAAGTCCGCCCCC | |
| | TATTGACGTCAATGACGGTAAATGGCCCGCCTGGCATTATGCCC | |
| | AGTACATGACCTTACGGGACTTTCCTACTTGGCAGTACATCTACG | |
| | TATTAGTCATCGCTATTACCATGGTGATGCGGTTTTGGCAGTACA | |
| | CCAATGGGCGTGGATAGCGGTTTGACTCACGGGGATTTCCAAGT | |
| | CTCCACCCCATTGACGTCAATGGGAGTTTGTTTTGGCACCAAAAT | |
| | CAACGGGACTTTCCAAAATGTCGTAATAACCCCGCCCCGTTGAC | |
| | GCAAATGGGCGGTAGGCGTGTACGGTGGGAGGTCTATATAAGCA | |
| | GAgGT | |
| 20 | ggccgccagtgtgatggatatctgcagaattcgcccttatggggatccgaacagagagacagcagaatatg | MND2 |
| | ggccaaacaggatatctgtggtaagcagttcctgccccggctcagggccaagaacagttggaacagcaga | |
| | atatgggccaaacaggatatctgtggtaagcagttcctgccccggctcagggccaagaacagatggtcccc | |
| | agatgcggtcccgccctcagcagtttctagagaaccatcagatgtttccagggtgccccaaggacctgaaat | |
| | gaccctgtgccttatttgaactaaccaatcagttcgcttctcgcttctgttcgcgcgcttctgctccccgagctct | |
| | atataagcagagctcgtttagtgaaccgtcagatcgcctggagacgccatccacgctgttttgacctccatag | |
| | aagacaccgactctaga | |
| 21 | GTAACGCCATTTTGCAAGGCatggaaaaataccaaaccaagaatagagaagttcagatc | SFFV |
| | aagggcgggtacatgaaaatagctaacgttgggccaaacaggatatctgcggtgagcagtttcggccccg | |
| | gcccggggccaagaacagatggtcaccgcagtttcggccccggcccgaggccaagaacagatggtccc | |
| | cagatatggcccaaccctcagcagtttcttaagacccatcagatgtttccaggctcccccaaggacctgaaat | |
| | gaccctgcgccttatttgaattaaccaatcagcctgcttctcgcttctgttcgcgcgcttctgcttcccgagctct | |
| | ataaaagagctcacaacccctcactcggcgcgccagtcctccGACAGACTGAGTCGCCCG | |
| | GG | |
| 22 | Gacattgtgctgacacagtctcctgcttccttagctgtatctctggggcagagggccaccatctcatgcagg | AAV-ScFv |
| | gccagccaaagtgtcagttcatctacctataattttatacactggtatcaacagaaatcaggacagccaccca | sequence |
| | aactcctcatcaagtatgcatccaacctagaatctggggtccctgtcaggttcagtggcagtgggtctgggac | (from D3 |
| | agacttctccctcaacatccatcctgtggaggaggaggatactgcaacatattactgtcagcacagttggga | antibody) |
| | gattccatacacgttcggagggggggccaagttggagataaaaggcagtactagcggtggtggctccggg | |
| | ggcggttccggtgggggcggcagcagccaggtgcagctgaaggagtcaggacctggcctggtggcacc | |
| | ctcacagagcctgtccatcacatgcaccgtctcaggattctcattaagcggctatggcataagctgggttcgc | |
| | cagcctccaggaaagcgtctggaatggctgggattgatatggggtgatggaaccacagactataattcagct | |
| | ctcaaatccagactgagcatcagcaaggacaactccaagagccaggttttcttaaaaatgaacagtctgcaa | |
| | actgatgacacagccaggtacttctgtaccagagggcctccggccttctataagtacctctactttgactactg | |
| | gggccaaggcaccactctcacagtctcctca | |
| 23 | gacaaaactcacacatgcccaccgtgcccagcacctcctgtggcaggaccgtcagtcttcctcttcccccca | Long hinge |
| | aaacccaaggacaccctcatgatctcccggacccctgaggtcacatgcgtggtggtggacgtgagccacg | (IgG1 |
| | aagaccctgaggtcaagttcaactggtacgtggacggcgtggaggtgcataatgccaagacaaagccgcg | hinge-IgG2 |
| | ggaggagcagtaccagagcacgtaccgtgtggtcagcgtcctcaccgtcctgcaccaggactggctgaat | hinge, |
| | ggcaaggagtacaagtgcaaggtctccaacaaagccctcccagcccccatcgagaaaaccatctccaaag | IgG1 CH2CH3) |
| | ccaaagggcagccccgagaaccacaggtgtacaccctgccaccatcacgagatgagctgaccaagaacc | |
| | aggtcagcctgacctgcctggtcaaaggcttctatcccagcgacatcgccgtggagtgggagagcaatgg | |
| | gcagccggagaacaactacaagaccacgcctcccgtgctggactccgacggctccttcttcctctacagca | |
| | agctcaccgtggacaagagcaggtggcagcaggggaacgtcttctcatgctccgtgatgcatgaggctctg | |
| | cacaaccactacacgcagaagagcctctccctgtctccc | |
| 24 | ATTGAAGTTATGTATCCTCCTCCTTACCTAGACAATGAGAAGAGC | Short Hinge |
| | AATGGAACCATTATCCATGTGAAAGGGAAACACCTTTGTCCAAG | (CD28 |
| | TCCCCTATTTCCCGGACCTTCTAAGCCC | extracellular |
| | | domain) |
| 25 | TTTTGGGTGCTGGTGGTGGTTGGTGGAGTCCTGGCTTGCTATAGC | Transmembrane |
| | TTGCTAGTAACAGTGGCCTTTATTATTTTCTGGGTG | (CD28 |
| | | transmembrane |
| | | domain) |
| 26 | Aggagtaagaggagcaggggaggtcacagtgactacatgaacatgactccccgccgccccgggcccac | Human CD28 |
| | ccgcaagcattaccagccctatgccccaccacgcgacttcgcagcctatcgctcc | signaling |
| | | domain |
| 27 | AACAGCAGAAGAAACAGAGGCGGCCAGAGCGACTACATGAACA | Murine CD28 |
| | TGACCCCCAGAAGGCCAGGCCTGACCAGAAAGCCCTACCAGCCC | signaling |
| | TACGCCCCTGCCAGAGACTTCGCCGCCTACAGACCC | domain |
| 28 | Aaacggggcagaaagaaactcctgtatatattcaaacaaccatttatgagaccagtacaaactactcaaga | Human 4-1BB |
| | ggaagatggctgtagctgccgatttccagaagaagaagaaggaggatgtgaactg | signaling |
| | | domain |
| 29 | TCTGTGCTCAAATGGATCAGGAAAAAATTCCCCCACATATTCAA | Murine 4-1BB |
| | GCAACCATTTAAGAAGACCACTGGAGCAGCTCAAGAGGAAGAT | signaling |
| | GCTTGTAGCTGCCGATGTCCACAGGAAGAAGAAGGAGGAGGAG | domain |
| | GAGGCTATGAGCTG | |
| 30 | Agagtgaagttcagcaggagcgcagacgcccccgcgtaccagcagggccagaaccagctctataacga | Human CD3z |
| | gctcaatctaggacgaagagaggagtacgatgttttggacaagagacgtggccgggaccctgagatggg | |
| | gggaaagccgagaaggaagaaccctcaggaaggcctgtacaatgaactgcagaaagataagatggcgg | |
| | aggcctacagtgagattgggatgaaaggcgagcgccggaggggcaaggggcacgatggcctttaccag | |
| | ggtctcagtacagccaccaaggacacctacgacgcccttcacatgcaggccctgccccctcgc | |
| 31 | AGAGCCAAGTTCAGCAGATCCGCCGAGACAGCCGCCAACCTGCA | Murine CD3z |
| | GGATCCCAACCAGCTGTACAACGAGCTGAACCTGGGCAGACGGG | |
| | AGGAATACGACGTGCTGGAAAAGAAGAGAGCCAGGGACCCCGA | |
| | GATGGGCGGCAAGCAGCAGAGAAGAAGAAACCCTCAGGAAGGC | |
| | GTCTACAACGCCCTGCAGAAAGACAAGATGGCCGAGGCCTACAG | |
| | CGAGATCGGCACCAAGGGCGAGAGAAGAAGGGGCAAGGGCCAC | |
| | GATGGCCTGTACCAGGGCCTGTCCACCGCCACCAAGGACACCTA | |
| | CGACGCCCTGCACATGCAGACCCTGGCCCCCAGATGA | |
| 32 | Atgccacctcctcgcctcctcttcttcctcctcttcctcacccccatggaagtcaggcccgaggaacctctag | CD19 |
| | tggtgaaggtggaagagggagataacgctgtgctgcagtgcctcaaggggacctcagatggccccactca | extracellular |
| | gcagctgacctggtctcgggagtccccgcttaaacccttcttaaaactcagcctggggctgccaggcctgg | domain |
| | gaatccacatgaggcccctggccatctggcttttcatcttcaacgtctctcaacagatggggggcttctacctg | |
| | tgccagccggggcccccctctgagaaggcctggcagcctggctggacagtcaatgtggagggcagcgg | |
| | ggagctgttccggtggaatgtttcggacctaggtggcctgggctgtggcctgaagaacaggtcctcagagg | |
| | gccccagctccccttccgggaagctcatgagccccaagctgtatgtgtgggccaaagaccgccctgagat | |
| | ctgggagggagagcctccgtgtgtcccaccgagggacagcctgaaccagagcctcagccaggacctcac | |
| | catggcccctggctccacactctggctgtcctgtggggtaccccctgactctgtgtccaggggccccctctc | |
| | ctggacccatgtgcaccccaaggggcctaagtcattgctgagcctagagctgaaggacgatcgcccggcc | |
| | agagatatgtgggtaatggagacgggtctgttgttgccccgggccacagctcaagacgctggaaagtattat | |
| | tgtcaccgtggcaacctgaccatgtcattccacctggagatcactgctcggccagtactatggcactggctg | |
| | ctgaggactggtggctggaaggtctcagctgtgactttggcttatctgatcttctgcctgtgttcccttgtgggc | |
| | attcttcatcttcaaagagccctggtcctgaggaggaaaaga | |
| 33 | Atgcttctcctggtgacaagccttctgctctgtgagttaccacacccagcattcctcctgatcccacgcaaagt | EGFR |
| | gtgtaacggaataggtattggtgaatttaaagactcactctccataaatgctacgaatattaaacacttcaaaaa | extracellular |
| | ctgcacctccatcagtggcgatctccacatcctgccggtggcatttaggggtgactccttcacacatactcctc | domain |
| | ctctggatccacaggaactggatattctgaaaaccgtaaaggaaatcacagggtattgctgattcaggcttg | |
| | gcctgaaaacaggacggacctccatgcctttgagaacctagaaatcatacgcggcaggaccaagcaacat | |
| | ggtcagttttctcttgcagtcgtcagcctgaacataacatccttgggattacgctccctcaaggagataagtga | |
| | tggagatgtgataatttcaggaaacaaaaatttgtgctatgcaaatacaataaactggaaaaaactgtttggga | |
| | cctccggtcagaaaaccaaaattataagcaacagaggtgaaaacagctgcaaggccacaggccaggtctg | |
| | ccatgccttgtgctcccccgagggctgctggggcccggagcccagggactgcgtctcttgccggaatgtca | |
| | gccgaggcagggaatgcgtggacaagtgcaaccttctggagggtgagccaagggagtttgtggagaact | |
| | ctgagtgcatacagtgccacccagagtgcctgcctcaggccatgaacatcacctgcacaggacggggacc | |
| | agacaactgtatccagtgtgcccactacattgacggcccccactgcgtcaagacctgcccggcaggagtca | |
| | tgggagaaaacaacaccctggtctggaagtacgcagacgccggccatgtgtgccacctgtgccatccaaa | |
| | ctgcacctacggatgcactgggccaggtcttgaaggctgtccaacgaatgggcctaagatcccgtccatcg | |
| | ccactggatctagat | |
| 34 | Cagtgtactaattatgctctcagaaattggctggagatgagagagcaaccccgggccg | E2A |
| 35 | Gaaggtcgtggatcactacttacgtgcggtgatgtagaagagaatccgggtccg | T2A |
| 36 | Gccacaaacactctctgctaaagcaagcaggtgatgagaagaaaaccccgggcct | P2A |
| 37 | atggctgccgatggttatcaccagattggctcgaggacaacctctctgagggcattcgcgagtggtgggcg | AAV8-capsid |
| | ctgaaacctggagccccgaagcccaaagccaaccagcaaaagcaggacgacggccggggtctggtgct | sequence |
| | tcctggctacaagtacctcggacccacaacggactcgacaagggggagcccgtcaacgcggcggacgc | |
| | agcggccctcgagcacgacaaggcctacgaccagcagctgcaggcgggtgacaatccgtacctgcggta | |
| | taaccacgccgacgccgagatcaggagcgtctgcaagaagatacgtcattgggggcaacctcgggcga | |
| | gcagtcaccaggccaagaagcgggactcgaacctctcggtctggagaggaaggcgctaagacggctcc | |
| | tggaaagaagagaccggtagagccatcaccccagcgactccagactcctctacgggcatcggcaagaaa | |
| | ggccaacagcccgccagaaaaagactcaattaggtcagactggcgactcagagtcagaccagaccctca | |
| | acctctcggagaacctccagcagcgccctctggtgtgggacctaatacaatggctgcaggcggtggcgca | |
| | ccaatggcagacaataacgaaggcgccgacggagtgggtagacctcgggaaattggcattgcgattcca | |
| | catggctgggcgacagagtcatcaccaccagcacccgaacctgggccctgcccacctacaacaaccacct | |
| | ctacaagcaaatctccaacgggacatcgggaggagccaccaacgacaacacctacttcggctacagcacc | |
| | ccctgggggtattagacataacagattccactgccactatcaccacgtgactggcagcgactcatcaacaa | |
| | caactggggattccggcccaagagactcagcttcaagctcacaacatccaggtcaaggaggtcacgcaga | |
| | atgaaggcaccaagaccatcgccaataacctcaccagcaccatccaggtgatacggactcggagtaccag | |
| | ctgccgtacgactcggctctgcccaccagggctgcctgcctccgacccggcggacgtgacatgattcccc | |
| | agtacggctacctaacactcaacaacggtagtcaggccgtgggacgctcctccactactgcctggaatactt | |
| | tccacgcagatgctgagaaccggcaacaacaccagatacttacaccacgaggacgtgccatccacagc | |
| | agctacgcccacagccagagcaggaccggctgatgaatcctctgattgaccagtacctgtactacttgtctc | |
| | ggactcaaacaacaggaggcacggcaaatacgcagactctgggcttcagccaaggtgggcctaatacaat | |
| | ggccaatcaggcaaagaactggctgccaggaccctgaaccgccaacaacgcgtctcaacgacaaccgg | |
| | gcaaaacaacaatagcaactagcctggactgctgggaccaaataccatctgaatggaagaaattcattggc | |
| | taatcctggcatcgctatggcaacacacaaagacgacgaggagcgattatcccagtaacgggatcctgatt | |
| | taggcaaacaaaatgctgccagagacaatgcggattacagcgatgtcatgctcaccagcgaggaagaaat | |
| | caaaaccactaaccctgtggctacagaggaatacggtatcgtggcagataacttgcagcagcaaaacacg | |
| | gctcctcaaattggaactgtcaacagccagggggccttacccggtatggtctggcagaaccgggacgtgta | |
| | cctgcagggtcccatctgggccaagattcctcacacggacggcaacaccacccgtctccgctgatgggcg | |
| | gctaggcctgaaacatcctccgcctcagatcctgatcaagaacacgcctgtacctgcggatcctccgacca | |
| | ccttcaaccagtcaaagctgaactcatcatcacgcaatacagcaccggacaggtcagcgtggaaattgaat | |
| | gggagctgcagaaggaaaacagcaagcgctggaaccccgagatccagtacacctccaactactacaaat | |
| | ctacaagtgtggactagctgaaatacagaaggcgtgtactctgaaccccgccccattggcacccgttacct | |
| | cacccgtaatctg | |
| 38 | atggctgccgatggttatcaccagattggctcgaggacaaccttagtgaaggaattcgcgagtggtgggctt | AAV9-capsid |
| | tgaaacctggagcccctcaacccaaggcaaatcaacaacatcaagacaacgctcgaggtcagtgcaccg | sequence |
| | ggttacaaataccaggacccggcaacggactcgacaagggggagccggtcaacgcagcagacgcggc | |
| | ggccctcgagcacgacaaggcctacgaccagcagctcaaggccggagacaacccgtacctcaagtaca | |
| | accacgccgacgccgagaccaggagcggctcaaagaagatacgtcattgggggcaacctcgggcgag | |
| | cagtcaccaggccaaaaagaggcacttgaacctcaggtctggagaggaagcggctaagacggctcctg | |
| | gaaagaagaggcctgtagagcagtctcctcaggaaccggactcctccgcgggtattggcaaatcgggtgc | |
| | acagcccgctaaaaagagactcaatacggtcagactggcgacacagagtcagtcccagaccctcaacca | |
| | atcggagaacctcccgcagccccctcaggtgtgggatctatacaatggatcaggtggtggcgcaccagt | |
| | ggcagacaataacgaaggtgccgatggagtgggtagacctcgggaaattggcattgcgattcccaatggc | |
| | tgggggacagagtcatcaccaccagcacccgaacctgggccctgcccacctacaacaatcacctctacaa | |
| | gcaaatctccaacagcacatctggaggatcttcaaatgacaacgcctacttcggctacagcaccccctggg | |
| | ggtattttgacttcaacagattccactgccacttctcaccacgtgactggcagcgactcatcaacaacaactg | |
| | gggattccggcctaagcgactcaacttcaagctcttcaacattcaggtcaaagaggttacggacaacaatgg | |
| | agtcaagaccatcgccaataaccttaccagcacggtccaggtcttcacggactcagactatcagctcccgta | |
| | cgtgctcgggtcggctcacgagggctgcctcccgccgttcccagcggacgttttcatgattcctcagtacgg | |
| | gtatctgacgcttaatgatggaagccaggccgtgggtcgttcgtccttttactgcctggaatatttcccgtcgc | |
| | aaatgctaagaacgggtaacaacttccagttcagctacgagtttgagaacgtacctttccatagcagctacgc | |
| | tcacagccaaagcctggaccgactaatgaatccactcatcgaccaatacttgtactatctctcaaagactatta | |
| | acggttctggacagaatcaacaaacgctaaaattcagtgtggccggacccagcaacatggctgtccaggg | |
| | aagaaactacatacctggacccagctaccgacaacaacgtgtctcaaccactgtgactcaaaacaacaaca | |
| | gcgaatttgcttggcctggagcttcttcttgggctctcaatggacgtaatagcttgatgaatcctggacctgcta | |
| | tggccagccacaaagaaggagaggaccgtttctttcctttgtctggatctttaatttttggcaaacaaggaact | |
| | ggaagagacaacgtggatgcggacaaagtcatgataaccaacgaagaagaaattaaaactactaacccgg | |
| | tagcaacggagtcctatggacaagtggccacaaaccaccagagtgcccaagcacaggcgcagaccggct | |
| | gggttcaaaaccaaggaatacttccgggtatggtttggcaggacagagatgtgtacctgcaaggacccattt | |
| | gggccaaaattcctcacacggacggcaactttcacccttctccgctgatgggagggtttggaatgaagcacc | |
| | cgcctcctcagatcctcatcaaaaacacacctgtacctgcggatcctccaacggccttcaacaaggacaag | |
| | ctgaactctttcatcacccagtattctactggccaagtcagcgtggagatcgagtgggagctgcagaaggaa | |
| | aacagcaagc | |
| 39 | DIVLTQSPASLAVSLGQRATISCRASQSVSSSTYNFIHWYQQKSGQP | Translated |
| | PKLLIKYASNLESGVPVRFSGSGSGTDFSLNIHPVEEEDTATYYCQH | AAV-ScFv |
| | SWEIPYTFGGGAKLEIKGSTSGGGSGGGSGGGGSSQVQLKESGPGL | sequence |
| | VAPSQSLSITCTVSGFSLSGYGISWVRQPPGKRLEWLGLIWGDGTTD | (from D3 |
| | YNSALKSRLSISKDNSKSQVFLKMNSLQTDDTARYFCTRGPPAFYK | antibody) |
| | YLYFDYWGQGTTLTVSS | |

*Unless otherwise specified, nucleic acid sequences are described 5′ to 3′ and amino acid sequences are described N-terminus to C-terminus

### EQUIVALENTS AND SCOPE

It is to be understood that this disclosure is not limited to any or all of the particular embodiments described expressly herein, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents (i.e., any lexicographical definition in the publications and patents cited that is not also expressly repeated in the disclosure should not be treated as such and should not be read as defining any terms appearing in the accompanying claims). If there is a conflict between any of the incorporated references and this disclosure, this disclosure shall control. In addition, any particular embodiment of this disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

In the claims articles such as “a,” “an,” and “the” may mean one or more than one unless indicated to the contrary or otherwise evident from the context. Wherever used herein, a pronoun in a gender (e.g., masculine, feminine, neuter, other, etc. . . . ) the pronoun shall be construed as gender neutral (i.e., construed to refer to all genders equally) regardless of the implied gender unless the context clearly indicates or requires otherwise. Wherever used herein, words used in the singular include the plural, and words used in the plural includes the singular, unless the context clearly indicates or requires otherwise. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists (e.g., in Markush group format), each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included in such ranges unless otherwise specified. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

Each element disclosed in the disclosure may be replaced by an alternative feature serving the same, equivalent, or similar purpose. Thus, unless expressly stated otherwise, each feature disclosed is only an example of a generic series of equivalent or similar features.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the disclosure, as defined in the following claims.

1-30. (canceled)

31. A method for modulating an immune response of a subject to an AAV capsid protein, the method comprising administering to the subject cell comprising a chimeric antigen receptor (CAR), wherein the CAR comprises:
(i) an antigen binding domain that targets (e.g., specifically binds) one or more Adeno-associated virus (AAV) capsid protein epitopes;
(ii) a transmembrane domain; and
(iii) a cytoplasmic signaling domain.

32. The method of claim 31, wherein the subject is a human.

33. The method of claim 31, wherein the subject has previously been administered an recombinant AAV (rAAV), or is contemplated to be administered an rAAV.

34. The method of claim 31, wherein the cell is a recombinant T-cell comprising the CAR, and the immune response of the subject to the AAV capsid protein is inhibited.

35. The method of claim 31, wherein the cell is a recombinant T-regulatory cell comprising the CAR, and the immune response of the subject to the AAV capsid protein is induced.

36. The method of claim 31, further comprising administering a therapeutic rAAV to the subject.

37. The method of claim 36, wherein the therapeutic rAAV comprises a capsid protein having the same serotype as the capsid protein targeted by the CAR.

38-42. (canceled)


SEQUENCE LISTING (39 sequences)

SEQ ID NO: 1 - DNA, 11147 bp, unidentified
  source 1..11147: mol_type = genomic DNA; note = AAV-CAR-FoxP3-EGFRt
ctgttttaaaagaaaaggggggattggggggtacagtgcaggggaaagaatagtagacataatagcaacagacatacaaactaaagaattacaaaaacaaattacaaaaattcaaaattttatcaacaagcttgatcgatggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcggatccacgcgtgcggccgcacgcgtaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctcgccgccaccatggctctgcccgtcaccgcactgctgctgcctctggctctgctgctgcacgccgcaagaccagacattgtgctgacacagtctcctgcttccttagctgtatctctggggcagagggccaccatctcatgcagggccagccaaagtgtcagttcatctacctataattttatacactggtatcaacagaaatcaggacagccacccaaactcctcatcaagtatgcatccaacctagaatctggggtccctgtcaggttcagtggcagtgggtctgggacagacttctccctcaacatccatcctgtggaggaggaggatactgcaacatattactgtcagcacagttgggagattccatacacgttcggagggggggccaagttggagataaaaggcagtactagcggtggtggctccgggggcggttccggtgggggcggcagcagccaggtgcagctgaaggagtcaggacctggcctggtggcaccctcacagagcctgtccatcacatgcaccgtctcaggattctcattaagcggctatggcataagctgggttcgccagcctccaggaaagcgtctggaatggctgggattgatatggggtgatggaaccacagactataattcagctctcaaatccagactgagcatcagcaaggacaactccaagagccaggttttcttaaaaatgaacagtctgcaaactgatgacacagccaggtacttctgtaccagagggcctccggccttctataagtacctctactttgactactggggccaaggcaccactctcacagtctcctcagacaaaactcacacatgcccaccgtgcccagcacctcctgtggcaggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgccaccatcacgagatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccttttgggtgctggtggtggttggtggagtcctggcttgctatagcttgctagtaacagtggcctttattattttctgggtgaggagtaagaggagcaggggaggtcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgtactaattatgctctcttgaaattggctggagatgttgagagcaaccccgggccggaattcatgcccaaccctaggccagccaagcctatggctccttccttggcccttggcccatccccaggagtcttgccaagctggaagactgcacccaagggctcagaacttctagggaccaggggctctgggggacccttccaaggtcgggacctgcgaagtggggcccacacctcttcttccttgaaccccctgccaccatcccagctgcagctgcctacagtgcccctagtcatggtggcaccgtctggggcccgactaggtccctcaccccacctacaggcccttctccaggacagaccacacttcatgcatcagctctccactgtggatgcccatgcccagacccctgtgctccaagtgcgtccactggacaacccagccatgatcagcctcccaccaccttctgctgccactggggtcttctccctcaaggcccggcctggcctgccacctgggatcaatgtggccagtctggaatgggtgtccagggagccagctctactctgcaccttcccacgctcgggtacacccaggaaagacagcaaccttttggctgcaccccaaggatcctacccactgctggcaaatggagtctgcaagtggcctggttgtgagaaggtcttcgaggagccagaagagtttctcaagcactgccaagcagatcatctcctggatgagaaaggcaaggcccagtgcctcctccagagagaagtggtgcagtctctggagcagcagctggagctggaaaaggagaagctgggagctatgcaggcccacctggctgggaagatggcgctggccaaggctccatctgtggcctcaatggacaagagctcttgctgcatcgtagccaccagtactcagggcagtgtgctcccggcctggtctgctcctcgggaggctccagacggcggcctgtttgcagtgcggaggcacctctggggaagccatggcaatagttccttcccagagttcttccacaacatggactacttcaagtaccacaatatgcgaccccctttcacctatgccacccttatccgatgggccatcctggaagccccggagaggcagaggacactcaatgaaatctaccattggtttactcgcatgttcgcctacttcagaaaccaccccgccacctggaagaatgccatccgccacaacctgagcctgcacaagtgctttgtgcgagtggagagcgagaagggagcagtgtggaccgtagatgaatttgagtttcgcaagaagaggagccaacgccccaacaagtgctccaatccctgccctgaattctctagacagtgtactaattatgctctcttgaaattggctggagatgttgagagcaaccccgggccgatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactggatctagataaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttcctcgagggggggcccggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggtt

SEQ ID NO: 2 - DNA, 10047 bp, unidentified
  source 1..10047: mol_type = genomic DNA; note = 14-2_Lenti-IF5-D3-NQ-28-BB-Z
ctgttttaaaagaaaaggggggattggggggtacagtgcaggggaaagaatagtagacataatagcaacagacatacaaactaaagaattacaaaaacaaattacaaaaattcaaaattttatcaacaagcttgatcgatggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcggatccacgcgtgcggccgcacgcgtaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctcgccgccaccatggagacagacacactcctgctatgggtgctgctgctctgggttccaggttccactggtgacattgtgctgacacagtctcctgcttccttagctgtatctctggggcagagggccaccatctcatgcagggccagccaaagtgtcagttcatctacctataattttatacactggtatcaacagaaatcaggacagccacccaaactcctcatcaagtatgcatccaacctagaatctggggtccctgtcaggttcagtggcagtgggtctgggacagacttctccctcaacatccatcctgtggaggaggaggatactgcaacatattactgtcagcacagttgggagattccatacacgttcggagggggggccaagttggagataaaaggcagtactagcggtggtggctccgggggcggttccggtgggggcggcagcagccaggtgcagctgaaggagtcaggacctggcctggtggcaccctcacagagcctgtccatcacatgcaccgtctcaggattctcattaagcggctatggcataagctgggttcgccagcctccaggaaagcgtctggaatggctgggattgatatggggtgatggaaccacagactataattcagctctcaaatccagactgagcatcagcaaggacaactccaagagccaggttttcttaaaaatgaacagtctgcaaactgatgacacagccaggtacttctgtaccagagggcctccggccttctataagtacctctactttgactactggggccaaggcaccactctcacagtctcctcagacaaaactcacacatgcccaccgtgcccagcacctcctgtggcaggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgccaccatcacgagatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccttttgggtgctggtggtggttggtggagtcctggcttgctatagcttgctagtaacagtggcctttattattttctgggtgaggagtaagaggagcaggggaggtcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgtactaattatgctctcttgaaattggctggagatgttgagagcaaccccgggccgatgcccaaccctaggccagccaagcctatggctccttccttggcccttggcccatccccaggagtcttgccaagctggaagactgcacccaagggctcagaacttctagggaccaggggctctgggggacccttccaaggtcgggacctgcgaagtggggcccacacctcttcttccttgaaccccctgccaccatcccagctgcagctgcctacagtgcccctagtcatggtggcaccgtctggggcccgactaggtccctcaccccacctacaggcccttctccaggacagaccacacttcatgcatcagctctccactgtggatgcccatgcccagacccctgtgctccaagtgcgtccactggacaacccagccatgatcagcctcccaccaccttctgctgccactggggtcttctccctcaaggcccggcctggcctgccacctgggatcaatgtggccagtctggaatgggtgtccagggagccagctctactctgcaccttcccacgctcgggtacacccaggaaagacagcaaccttttggctgcaccccaaggatcctacccactgctggcaaatggagtctgcaagtggcctggttgtgagaaggtcttcgaggagccagaagagtttctcaagcactgccaagcagatcatctcctggatgagaaaggcaaggcccagtgcctcctccagagagaagtggtgcagtctctggagcagcagctggagctggaaaaggagaagctgggagctatgcaggcccacctggctgggaagatggcgctggccaaggctccatctgtggcctcaatggacaagagctcttgctgcatcgtagccaccagtactcagggcagtgtgctcccggcctggtctgctcctcgggaggctccagacggcggcctgtttgcagtgcggaggcacctctggggaagccatggcaatagttccttcccagagttcttccacaacatggactacttcaagtaccacaatatgcgaccccctttcacctatgccacccttatccgatgggccatcctggaagccccggagaggcagaggacactcaatgaaatctaccattggtttactcgcatgttcgcctacttcagaaaccaccccgccacctggaagaatgccatccgccacaacctgagcctgcacaagtgctttgtgcgagtggagagcgagaagggagcagtgtggaccgtagatgaatttgagtttcgcaagaagaggagccaacgccccaacaagtgctccaatccctgcccttgaaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttcctcgagggggggcccggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggtt

SEQ ID NO: 3 - DNA, 9836 bp, unidentified
  source 1..9836: mol_type = genomic DNA; note = CD8leader-AAV-CAR-2A-EGFRt
ctgttttaaaagaaaaggggggattggggggtacagtgcaggggaaagaatagtagacataatagcaacagacatacaaactaaagaattacaaaaacaaattacaaaaattcaaaattttatcaacaagcttgatcgatggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcggatccacgcgtgcggccgcacgcgtaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctcgccgccaccatggctctgcccgtcaccgcactgctgctgcctctggctctgctgctgcacgccgcaagaccagacattgtgctgacacagtctcctgcttccttagctgtatctctggggcagagggccaccatctcatgcagggccagccaaagtgtcagttcatctacctataattttatacactggtatcaacagaaatcaggacagccacccaaactcctcatcaagtatgcatccaacctagaatctggggtccctgtcaggttcagtggcagtgggtctgggacagacttctccctcaacatccatcctgtggaggaggaggatactgcaacatattactgtcagcacagttgggagattccatacacgttcggagggggggccaagttggagataaaaggcagtactagcggtggtggctccgggggcggttccggtgggggcggcagcagccaggtgcagctgaaggagtcaggacctggcctggtggcaccctcacagagcctgtccatcacatgcaccgtctcaggattctcattaagcggctatggcataagctgggttcgccagcctccaggaaagcgtctggaatggctgggattgatatggggtgatggaaccacagactataattcagctctcaaatccagactgagcatcagcaaggacaactccaagagccaggttttcttaaaaatgaacagtctgcaaactgatgacacagccaggtacttctgtaccagagggcctccggccttctataagtacctctactttgactactggggccaaggcaccactctcacagtctcctcagacaaaactcacacatgcccaccgtgcccagcacctcctgtggcaggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgccaccatcacgagatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccttttgggtgctggtggtggttggtggagtcctggcttgctatagcttgctagtaacagtggcctttattattttctgggtgaggagtaagaggagcaggggaggtcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgtactaattatgctctcttgaaattggctggagatgttgagagcaaccccgggccgatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgaggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttcctcgagggggggcccggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggtt

SEQ ID NO: 4 - DNA, 8711 bp, unidentified
  source 1..8711: mol_type = genomic DNA; note = 13-1_Lenti-IF5-D3-NQ-28-BB-Z
ctgttttaaaagaaaaggggggattggggggtacagtgcaggggaaagaatagtagacataatagcaacagacatacaaactaaagaattacaaaaacaaattacaaaaattcaaaattttatcaacaagcttgatcgatggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcggatccacgcgtgcggccgcacgcgtaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctcgccgccaccatggagacagacacactcctgctatgggtgctgctgctctgggttccaggttccactggtgacattgtgctgacacagtctcctgcttccttagctgtatctctggggcagagggccaccatctcatgcagggccagccaaagtgtcagttcatctacctataattttatacactggtatcaacagaaatcaggacagccacccaaactcctcatcaagtatgcatccaacctagaatctggggtccctgtcaggttcagtggcagtgggtctgggacagacttctccctcaacatccatcctgtggaggaggaggatactgcaacatattactgtcagcacagttgggagattccatacacgttcggagggggggccaagttggagataaaaggcagtactagcggtggtggctccgggggcggttccggtgggggcggcagcagccaggtgcagctgaaggagtcaggacctggcctggtggcaccctcacagagcctgtccatcacatgcaccgtctcaggattctcattaagcggctatggcataagctgggttcgccagcctccaggaaagcgtctggaatggctgggattgatatggggtgatggaaccacagactataattcagctctcaaatccagactgagcatcagcaaggacaactccaagagccaggttttcttaaaaatgaacagtctgcaaactgatgacacagccaggtacttctgtaccagagggcctccggccttctataagtacctctactttgactactggggccaaggcaccactctcacagtctcctcagacaaaactcacacatgcccaccgtgcccagcacctcctgtggcaggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgccaccatcacgagatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccttttgggtgctggtggtggttggtggagtcctggcttgctatagcttgctagtaacagtggcctttattattttctgggtgaggagtaagaggagcaggggaggtcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgagatatcgtgggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttcctcgagggggggcccggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggtt

SEQ ID NO: 5 - DNA, 5054 bp, unidentified
  source 1..5054: mol_type = genomic DNA; note = EF1alpha
gtcgacattgattattgactagatcatcgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgg

SEQ ID NO: 6 - DNA, 80 bp, unidentified
  source 1..80: mol_type = genomic DNA; note = Woodchuck Hepatitis Virus Posttranscriptional Regulatory Element Alpha Component
gccacggcggaactcatcgccgcctgccttgcccgctgctggacaggggctcggctgttgggcactgacaattccgtggt

SEQ ID NO: 7 - DNA, 592 bp, unidentified
  source 1..592: mol_type = genomic DNA; note = Woodchuck Hepatitis Virus Posttranscriptional Regulatory Element
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 8 - protein, 18 aa, unidentified
  REGION 1..18: note = T2A
EGRGSLLTCGDVEENPGP

SEQ ID NO: 9 - protein, 19 aa, unidentified
  REGION 1..19: note = P2A
ATNFSLLKQAGDVEENPGP

SEQ ID NO: 10 - protein, 20 aa, unidentified
  REGION 1..20: note = E2A
QCTNYALLKLAGDVESNPGP

SEQ ID NO: 11 - protein, 22 aa, unidentified
  REGION 1..22: note = F2A
VKQTLNFDLLKLAGDVESNPGP

SEQ ID NO: 12 - protein, 21 aa, unidentified
  REGION 1..21: note = T2A
GSGEGRGSLLTCGDVEENPGP

SEQ ID NO: 13 - protein, 22 aa, unidentified
  REGION 1..22: note = P2A
GSGATNFSLLKQAGDVEENPGP

SEQ ID NO: 14 - protein, 23 aa, unidentified
  REGION 1..23: note = E2A
GSGQCTNYALLKLAGDVESNPGP

SEQ ID NO: 15 - protein, 25 aa, unidentified
  REGION 1..25: note = F2A
GSGVKQTLNFDLLKLAGDVESNPGP

SEQ ID NO: 16 - DNA, 11111 bp, unidentified
  source 1..11111: mol_type = genomic DNA; note = 64-AAV-CAR-T-Regs
ctgttttaaaagaaaaggggggattggggggtacagtgcaggggaaagaatagtagacataatagcaacagacatacaaactaaagaattacaaaaacaaattacaaaaattcaaaattttatcaacaagcttgatcgatggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcggatccacgcgtgcggccgcacgcgtaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctcgccgccaccatggctctgcccgtcaccgcactgctgctgcctctggctctgctgctgcacgccgcaagaccagacattgtgctgacacagtctcctgcttccttagctgtatctctggggcagagggccaccatctcatgcagggccagccaaagtgtcagttcatctacctataattttatacactggtatcaacagaaatcaggacagccacccaaactcctcatcaagtatgcatccaacctagaatctggggtccctgtcaggttcagtggcagtgggtctgggacagacttctccctcaacatccatcctgtggaggaggaggatactgcaacatattactgtcagcacagttgggagattccatacacgttcggagggggggccaagttggagataaaaggcagtactagcggtggtggctccgggggcggttccggtgggggcggcagcagccaggtgcagctgaaggagtcaggacctggcctggtggcaccctcacagagcctgtccatcacatgcaccgtctcaggattctcattaagcggctatggcataagctgggttcgccagcctccaggaaagcgtctggaatggctgggattgatatggggtgatggaaccacagactataattcagctctcaaatccagactgagcatcagcaaggacaactccaagagccaggttttcttaaaaatgaacagtctgcaaactgatgacacagccaggtacttctgtaccagagggcctccggccttctataagtacctctactttgactactggggccaaggcaccactctcacagtctcctcactcgaccccaaatcttctgacaaaactcacacatgcccaccgtgcccagcacctcctgtggcaggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgccaccatcacgagatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccttttgggtgctggtggtggttggtggagtcctggcttgctatagcttgctagtaacagtggcctttattattttctgggtgaggagtaagaggagcaggggaggtcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgtactaattatgctctcttgaaattggctggagatgttgagagcaaccccgggccggaattcatgcccaaccctaggccagccaagcctatggctccttccttggcccttggcccatccccaggagtcttgccaagctggaagactgcacccaagggctcagaacttctagggaccaggggctctgggggacccttccaaggtcgggacctgcgaagtggggcccacacctcttcttccttgaaccccctgccaccatcccagctgcagctgcctacagtgcccctagtcatggtggcaccgtctggggcccgactaggtccctcaccccacctacaggcccttctccaggacagaccacacttcatgcatcagctctccactgtggatgcccatgcccagacccctgtgctccaagtgcgtccactggacaacccagccatgatcagcctcccaccaccttctgctgccactggggtcttctccctcaaggcccggcctggcctgccacctgggatcaatgtggccagtctggaatgggtgtccagggagccagctctactctgcaccttcccacgctcgggtacacccaggaaagacagcaaccttttggctgcaccccaaggatcctacccactgctggcaaatggagtctgcaagtggcctggttgtgagaaggtcttcgaggagccagaagagtttctcaagcactgccaagcagatcatctcctggatgagaaaggcaaggcccagtgcctcctccagagagaagtggtgcagtctctggagcagcagctggagctggaaaaggagaagctgggagctatgcaggcccacctggctgggaagatggcgctggccaaggctccatctgtggcctcaatggacaagagctcttgctgcatcgtagccaccagtactcagggcagtgtgctcccggcctggtctgctcctcgggaggctccagacggcggcctgtttgcagtgcggaggcacctctggggaagccatggcaatagttccttcccagagttcttccacaacatggactacttcaagtaccacaatatgcgaccccctttcacctatgccacccttatccgatgggccatcctggaagccccggagaggcagaggacactcaatgaaatctaccattggtttactcgcatgttcgcctacttcagaaaccaccccgccacctggaagaatgccatccgccacaacctgagcctgcacaagtgctttgtgcgagtggagagcgagaagggagcagtgtggaccgtagatgaatttgagtttcgcaagaagaggagccaacgccccaacaagtgctccaatccctgcccccagtgtactaattatgctctcttgaaattggctggagatgttgagagcaaccccgggccgatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagatgatctagataaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttcctcgagggggggcccggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggtt

SEQ ID NO: 17 - DNA, 9764 bp, unidentified
  source 1..9764: mol_type = genomic DNA; note = 65-AAV-CAR-T-Cells
ctgttttaaaagaaaaggggggattggggggtacagtgcaggggaaagaatagtagacataatagcaacagacatacaaactaaagaattacaaaaacaaattacaaaaattcaaaattttatcaacaagcttgatcgatggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcggatccacgcgtgcggccgcacgcgtaagcttagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctcgccgccaccatggctctgcccgtcaccgcactgctgctgcctctggctctgctgctgcacgccgcaagaccagacattgtgctgacacagtctcctgcttccttagctgtatctctggggcagagggccaccatctcatgcagggccagccaaagtgtcagttcatctacctataattttatacactggtatcaacagaaatcaggacagccacccaaactcctcatcaagtatgcatccaacctagaatctggggtccctgtcaggttcagtggcagtgggtctgggacagacttctccctcaacatccatcctgtggaggaggaggatactgcaacatattactgtcagcacagttgggagattccatacacgttcggagggggggccaagttggagataaaaggcagtactagcggtggtggctccgggggcggttccggtgggggcggcagcagccaggtgcagctgaaggagtcaggacctggcctggtggcaccctcacagagcctgtccatcacatgcaccgtctcaggattctcattaagcggctatggcataagctgggttcgccagcctccaggaaagcgtctggaatggctgggattgatatggggtgatggaaccacagactataattcagctctcaaatccagactgagcatcagcaaggacaactccaagagccaggttttcttaaaaatgaacagtctgcaaactgatgacacagccaggtacttctgtaccagagggcctccggccttctataagtacctctactttgactactggggccaaggcaccactctcacagtctcctcactcgaccccaaatcttctgacaaaactcacacatgcccaccgtgcccagcacctcctgtggcaggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgccaccatcacgagatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccttttgggtgctggtggtggttggtggagtcctggcttgctatagcttgctagtaacagtggcctttattattttctgggtgaggagtaagaggagcaggggaggtcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgtactaattatgctctcttgaaattggctggagatgttgagagcaaccccgggccggaattcatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagatgatctagataaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttcctcgagggggggcccggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggtt

SEQ ID NO: 18 - DNA, 291 bp, unidentified
  source 1..291: mol_type = genomic DNA; note = EF1-alpha
aacaagcttgatcgatggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcggatccacgcgtgcggccgcacgcgtaagctt

SEQ ID NO: 19 - DNA, 584 bp, unidentified
  source 1..584: mol_type = genomic DNA; note = CMV
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggt

SEQ ID NO: 20 - DNA, 450 bp, unidentified
  source 1..450: mol_type = genomic DNA; note = MND2
ggccgccagtgtgatggatatctgcagaattcgcccttatggggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctaga

SEQ ID NO: 21 - DNA, 408 bp, unidentified
  source 1..408: mol_type = genomic DNA; note = SFFV
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggg

SEQ ID NO: 22 - DNA, 750 bp, unidentified
  source 1..750: mol_type = genomic DNA; note = AAV-ScFv
gacattgtgctgacacagtctcctgcttccttagctgtatctctggggcagagggccaccatctcatgcagggccagccaaagtgtcagttcatctacctataattttatacactggtatcaacagaaatcaggacagccacccaaactcctcatcaagtatgcatccaacctagaatctggggtccctgtcaggttcagtggcagtgggtctgggacagacttctccctcaacatccatcctgtggaggaggaggatactgcaacatattactgtcagcacagttgggagattccatacacgttcggagggggggccaagttggagataaaaggcagtactagcggtggtggctccgggggcggttccggtgggggcggcagcagccaggtgcagctgaaggagtcaggacctggcctggtggcaccctcacagagcctgtccatcacatgcaccgtctcaggattctcattaagcggctatggcataagctgggttcgccagcctccaggaaagcgtctggaatggctgggattgatatggggtgatggaaccacagactataattcagctctcaaatccagactgagcatcagcaaggacaactccaagagccaggttttcttaaaaatgaacagtctgcaaactgatgacacagccaggtacttctgtaccagagggcctccggccttctataagtacctctactttgactactggggccaaggcaccactctcacagtctcctca

SEQ ID NO: 23 - DNA, 672 bp, unidentified
  source 1..672: mol_type = genomic DNA; note = IgG1 hinge-IgG2 hinge, IgG1 CH2CH3
gacaaaactcacacatgcccaccgtgcccagcacctcctgtggcaggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtaccagagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgccaccatcacgagatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccc

SEQ ID NO: 24 - DNA, 117 bp, unidentified
  source 1..117: mol_type = genomic DNA; note = CD28 extracellular domain
attgaagttatgtatcctcctccttacctagacaatgagaagagcaatggaaccattatccatgtgaaagggaaacacctttgtccaagtcccctatttcccggaccttctaagccc

SEQ ID NO: 25 - DNA, 81 bp, unidentified
  source 1..81: mol_type = genomic DNA; note = CD28 transmembrane domain
ttttgggtgctggtggtggttggtggagtcctggcttgctatagcttgctagtaacagtggcctttattattttctgggtg

SEQ ID NO: 26 - DNA, 123 bp, unidentified
  source 1..123: mol_type = genomic DNA; note = Human CD28 signaling domain
aggagtaagaggagcaggggaggtcacagtgactacatgaacatgactccccgccgccccgggcccacccgcaagcattaccagccctatgccccaccacgcgacttcgcagcctatcgctcc

SEQ ID NO: 27 - DNA, 123 bp, unidentified
  source 1..123: mol_type = genomic DNA; note = Murine CD28 signaling domain
aacagcagaagaaacagaggcggccagagcgactacatgaacatgacccccagaaggccaggcctgaccagaaagccctaccagccctacgcccctgccagagacttcgccgcctacagaccc

SEQ ID NO: 28 - DNA, 126 bp, unidentified
  source 1..126: mol_type = genomic DNA; note = Human 4-1BB signaling domain
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 29 - DNA, 144 bp, unidentified
  source 1..144: mol_type = genomic DNA; note = Murine 4-1BB signaling domain
tctgtgctcaaatggatcaggaaaaaattcccccacatattcaagcaaccatttaagaagaccactggagcagctcaagaggaagatgcttgtagctgccgatgtccacaggaagaagaaggaggaggaggaggctatgagctg

SEQ ID NO: 30 - DNA, 336 bp, unidentified
  source 1..336: mol_type = genomic DNA; note = Human CD3z
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 31 - DNA, 342 bp, unidentified
  source 1..342: mol_type = genomic DNA; note = Murine CD3z
agagccaagttcagcagatccgccgagacagccgccaacctgcaggatcccaaccagctgtacaacgagctgaacctgggcagacgggaggaatacgacgtgctggaaaagaagagagccagggaccccgagatgggcggcaagcagcagagaagaagaaaccctcaggaaggcgtctacaacgccctgcagaaagacaagatggccgaggcctacagcgagatcggcaccaagggcgagagaagaaggggcaagggccacgatggcctgtaccagggcctgtccaccgccaccaaggacacctacgacgccctgcacatgcagaccctggcccccagatga

SEQ ID NO: 32 - DNA, 969 bp, unidentified
  source 1..969: mol_type = genomic DNA; note = CD19 extracellular domain
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaaga

SEQ ID NO: 33 - DNA, 1021 bp, unidentified
  source 1..1021: mol_type = genomic DNA; note = EGFR extracellular domain
atgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactggatctagat

SEQ ID NO: 34 - DNA, 60 bp, unidentified
  source 1..60: mol_type = genomic DNA; note = E2A
cagtgtactaattatgctctcttgaaattggctggagatgttgagagcaaccccgggccg

SEQ ID NO: 35 - DNA, 54 bp, unidentified
  source 1..54: mol_type = genomic DNA; note = T2A
gaaggtcgtggatcactacttacgtgcggtgatgtagaagagaatccgggtccg

SEQ ID NO: 36 - DNA, 57 bp, unidentified
  source 1..57: mol_type = genomic DNA; note = P2A
gccacaaacttctctctgctaaagcaagcaggtgatgttgaagaaaaccccgggcct

SEQ ID NO: 37 - DNA, 2214 bp, unidentified
  source 1..2214: mol_type = genomic DNA; note = AAV8-capsid
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 38 - DNA, 2080 bp, unidentified
  source 1..2080: mol_type = genomic DNA; note = AAV9-capsid
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagc

SEQ ID NO: 39 - protein, 250 aa, unidentified
  source 1..250: note = AAV-ScFv
DIVLTQSPASLAVSLGQRATISCRASQSVSSSTYNFIHWYQQKSGQPPKLLIKYASNLESGVPVRFSGSGSGTDFSLNIHPVEEEDTATYYCQHSWEIPYTFGGGAKLEIKGSTSGGGSGGGSGGGGSSQVQLKESGPGLVAPSQSLSITCTVSGFSLSGYGISWVRQPPGKRLEWLGLIWGDGTTDYNSALKSRLSISKDNSKSQVFLKMNSLQTDDTARYFCTRGPPAFYKYLYFDYWGQGTTLTVSS